(19) United States
(12) Patent Application Publication
(10) Pub. No.: US 2022/0143059 A1
(43) Pub. Date: May 12, 2022

(54) COMPOSITIONS AND THERAPEUTIC METHODS

(71) Applicant: AyuVis Research, Inc., Fort Worth, TX (US)

(72) Inventors: Suchismita Acharya, Euless, TX (US); Sumita Behera, Flower Mound, TX (US)

(21) Appl. No.: 17/525,060

(22) Filed: Nov. 12, 2021

Related U.S. Application Data
(63) Continuation-in-part of application No. 15/473,904, filed on Mar. 30, 2017, now Pat. No. 11,207,343.
(60) Provisional application No. 62/315,144, filed on Mar. 30, 2016.

(51) Int. Cl. A61K 31/7056; A61K 45/06; A61K 31/7048; A61K 31/7034; A61K 31/7036; A61K 31/706; A61K 9/00; A61K 47/22; A61K 47/26; A61K 47/10; A61K 47/38; A61K 47/14; A61K 47/02; A61K 47/32; A61K 9/06

(52) U.S. Cl. CPC A61K 31/7056; A61K 9/06; A61K 31/7048; A61K 31/7034; A61K 31/7036; A61K 31/706; A61K 9/0053; A61K 47/22; A61K 47/26; A61K 47/10; A61K 47/38; A61K 47/14; A61K 47/02; A61K 47/32; A61K 45/06

(57) ABSTRACT

The present invention is directed to novel products, variants, pharmaceutically acceptable salts and prodrugs thereof, and medical use of such compounds for the treatment and/or management of sepsis, septicemia, septic shock, peritonitis, skin and soft tissue infections, ocular infection, ocular inflammation, ocular angiogenesis, rheumatoid arthritis (RA), atherosclerosis, inflammatory bowel diseases (IBD), necrotizing enterocolitis, asthma, chronic obstructive pulmonary disease, acute respiratory distress syndrome, acute lung injury, bacterial and viral pneumonia, chronic lung injury, acute kidney injury, chronic kidney injury, fibrosis, fever syndromes, cachexia, psoriasis, autoimmune diseases, cardiac diseases, retinoblastoma, cancer and/or any disorder associated with inflammation, immunomodulation and microbial infection.

### CROSS-REFERENCE TO RELATED APPLICATIONS

This application claims priority to and is a continuation-in-part application of U.S. application Ser. No. 15/473,904, filed Mar. 30, 2017, now U.S. Pat. No. 11,207,343, and claims priority to and claims the benefit of U.S. Provisional Application No. 62/315,144, filed Mar. 30, 2016, which application is incorporated herein by reference.

### TECHNICAL FIELD OF THE INVENTION

The present invention is directed to novel products, variants, pharmaceutically acceptable salts and prodrugs thereof, and medical use of such compounds for the treatment and/or management of sepsis, septicemia, septic shock, peritonitis, skin and soft tissue infections, ocular infection, ocular inflammation, ocular angiogenesis, rheumatoid arthritis (RA), atherosclerosis, inflammatory bowel diseases (IBD), necrotizing enterocolitis, asthma, chronic obstructive pulmonary disease, acute respiratory distress syndrome, acute lung injury, bacterial and viral pneumonia, chronic lung injury, acute kidney injury, chronic kidney injury, fibrosis, fever syndromes, cachexia, psoriasis, autoimmune diseases, cardiac diseases, retinoblastoma, cancer and/or any disorder associated with inflammation, immunomodulation and microbial infection.

### BACKGROUND OF THE INVENTION

Sepsis was identified as one of the five conditions that account for the most expensive hospital stays in the United States. The outcome of sepsis is particularly unfavorable in elderly, immunocompromised, and critically ill patients. Besides its clinical challenge, the treatment of sepsis imposes a large economic burden on healthcare systems worldwide. With an estimated >900,000 cases occurring in the United States alone each year, the annual total costs have been estimated to be approximately $26 billion nationally. Currently, one of three broad adjunctive (nonantibiotic) therapy approaches are typically used to treat sepsis: (1) improving supportive care (i.e. oxygenation/ventilation strategies, optimization of fluid/vasopressor use, early goal-directed therapy); (2) targeting bacterial virulence factors (i.e. antiendotoxin antibodies, endotoxin removal columns); and (3) targeting host response factors (i.e. corticosteroids, anticytokine drugs, anticoagulants). However, current therapies are not completely effective in patients with sepsis and septic shock. These therapies are even less effective in immunocompromised and older patients.

Hospital-acquired pneumonia (HAP) is a common healthcare-acquired infection, which occurs upon colonization by bacteria, most commonly Pseudomonas aeruginosa, following their entry into the lower respiratory tract, often by medical interventions such as ventilators or endotracheal intubation. The two pneumonia subgroups, non-ventilator HAP (NV-HAP) and ventilator-associated pneumonia (VAP), both cause substantial clinical and economic burdens, including prolonged hospital stay, higher healthcare costs, and increased morbidity and mortality. HAP occurs at a rate of 4-50 cases per 1,000 admissions in community hospitals and general medical wards, and 120-220 cases per 1,000 ICU admissions¹, accounting for >50% of the antibiotics prescribed in ICUs, and resulting in 33-50% mortality². P. aeruginosa-associated pneumonia (PAP) is the most common lung infection for patients on mechanical ventilation, with 50,000-60,000 cases in the US each year, a 13.5-50% mortality rate and increasing numbers of multi-drug resistant (MDR) strains³. Community acquired pneumonia (CAP) is also becoming a leading infectious cause of morbidity and mortality^{4,5}, resulting in severe illness, poor clinical outcomes 6-8, and >3.5 million deaths annually worldwide^{4,9}. Additionally, P. aeruginosa infects about 75% of patients with cystic fibrosis aged 25-35 years; the mucoid form of the infection has a poor prognosis.

This infection has been treated traditionally by combination of fluoroquinolone and aminoglycoside antibiotics such as ciprofloxacin and colistin, in combination with meropenem, as P. aeruginosa is developing resistant to β-lactam antibiotics¹⁰. A new generation β-lactam antibiotic cefepime seems more effective against P. aeruginosa; however, increasing incidence of antimicrobial resistance¹¹ has led to a serious restriction in treatment options for P. aeruginosa infections. This rise in antimicrobial resistance has led the World Health Organization to list P. aeruginosa as a “Priority 1: Critical” pathogen in need of research and development of new therapeutic approaches to treating infections¹².

To address the MDR problem, combination therapy is an emerging option. Combinations of two antibiotics, or of an antibiotic with a drug targeting the antibiotic resistance mechanism, or of an antibiotic with an immunostimulant vaccine adjuvant are promising new therapeutic approaches^{13,14}. The clinical manifestation of infections caused by the bacterial pathogens reflects a complex interaction between the pathogen, host, and antibiotics. In fact, endotoxin lipopolysaccharide (LPS), a component of outer membranes of gram-negative bacteria such as P. aeruginosa, has been reported to be responsible for the pathogenesis of acute organ injury and sepsis by provoking host inflammatory responses and inducing systemic inflammatory syndromes¹⁵. As the innate immune system plays a critical role in battling the bacterial infection, strategic targeting of the host together with an appropriate antimicrobial treatment of the pathogen(s) by rational combination therapy may suppress antibacterial resistance, lead to resolution of antimicrobial-resistant infections, and mitigate the damages caused by an overt inflammatory response, thereby overcoming some of the impediments to antibiotic therapies.

Acute Respiratory Distress Syndrome (ARDS) is a severe form of respiratory failure, leading to other organ failure, sepsis/death that develops in association with a variety of insults including massive hemorrhage, systemic infection, inhalation of noxious agents, bacterial infections, burns and blast trauma and has an overall mortality of 30-40%. It is estimated that 200,000 individuals develop ARDS each year in the United States (Rubenfeld, Caldwell et al. 2005, Villar, Blanco et al. 2016). One of the major complications of trauma-based morbidity in military combat is the development of ARDS, occurring in 8-82% of selected patient populations. These include patients with pulmonary contusions, severe trauma (Injury Severity Score >25), head injury, notable blood transfusion requirement, and major orthopedic injuries such as long-bone and pelvic fractures. The presence of ARDS is associated with a significant increase in morbidity, an increased use of hospital resources, and up to a 4.3-fold increase in mortality (Salim, Martin et al. 2006).

To date, there is no specific cure for ARDS. Current therapies only include supportive care, using a mechanical ventilator to support the lung until it fully recovers. Thus, new therapies for this disease are desperately needed.

Another such disease is Bronchopulmonary dysplasia (BPD), which is the most common chronic respiratory disease in infants and is a devastating condition that disrupts the developmental program of the lung secondary to preterm birth. BPD occurs secondary to an interaction between genetic and environmental factors (hyperoxia, invasive mechanical ventilation and sepsis) (Bhandari and Bhandari 2009, Bhandari and Bhandari 2011, Jensen and Schmidt 2014). Although the definition of BPD has evolved over the past decade, it is currently defined as the need for oxygen (O₂) supplementation for 28 days of life and a “physiologic” assessment of the supplemental O₂ requirement at 36 weeks postmenstrual age (Bhandari and Bhandari 2011, Trembath and Laughon 2012, Bancalari and Claure 2016). It is estimated that 10,000-15,000 new cases of BPD occur each year in the United States, and significantly, 97% of all BPD cases occur in infants with a birth weight less than 1250 grams (Bhandari and Bhandari 2011). Despite many advances in neonatal ventilation techniques, widespread use of surfactant and antenatal corticosteroids, as well as aggressive fluid management, the incidence of BPD has remained the same (Smith, Zupancic et al. 2005) or even increased slightly (Bhandari and Bhandari 2011, Trembath and Laughon 2012). Management of BPD takes a considerable toll on health services. Among preterm infants, the single costliest complication of hospitalization during infancy is BPD, with an average cost per discharge of $116,000 (Russell, Green et al. 2007). Additionally, BPD is associated with significant pulmonary and neurodevelopmental sequelae that continue to have health ramifications into adulthood (Bhandari and Bhandari 2011, Natarajan, Pappas et al. 2012, Bhandari and McGrath-Morrow 2013, Raju, Buist et al. 2017). It is thus important to understand the long-term consequences of BPD, as they are likely to have a significant impact on treatment and cost and application of health care during the lifetime of those born prematurely.

The pathologic hallmarks of BPD include: hyperoxia-induced pulmonary inflammation (Bhandari 2014, Balany and Bhandari 2015, Harijith and Bhandari 2016), increased cell death (Li, Choo-Wing et al. 2011, Choo-Wing R 2013, Sureshbabu, Syed et al. 2015, Sureshbabu, Syed et al. 2016), dysregulated angiogenic factors (Bhandari, Choo-Wing et al. 2008, Sun H 2013, Sun H 2013, Syed, Choo-Wing et al. 2016) culminating in impaired alveolarization and dysregulated vascularization of the lung (Balany and Bhandari 2015). Respiratory distress syndrome (RDS) and hyperoxia exposure are common antecedents of BPD. The current standard-of-care therapeutic approach to treat RDS in premature neonates is exogenous surfactant and supplemental oxygen, however, there is no specific and effective method of prevention or treatment of BPD (Bhandari 2014). According to the National Institute of Child Health and Human Development/National Heart, Lung and Blood Institute (NICHD/NHLBI), the identification of potential drugs to target BPD in preterm infants has been categorized as a “research priority” (McEvoy, Jain et al. 2014). The National Institutes of Health (NIH) workshop conducted under the auspices of the NHLBI of the NIH on the primary prevention of chronic lung diseases focused on BPD (McEvoy, Jain et al. 2014). In terms of “promising near-term opportunities for primary BPD prevention research,” specifically, “clinical research priorities and specific clinical trials for BPD prevention,” it was disappointing to note that only two specific drugs were named—caffeine and inhaled nitric oxide (iNO) (Bhandari 2014).

Reducing inflammation and maintaining the balance between inflammatory and anti-inflammatory molecules via producing alternatively activated macrophages will be therapeutic for acute lung injury (ALI), ARDS, sepsis, pneumonia and BPD, which are characterized by overproduction of pro-inflammatory cytokines TNF-α, IL-6, i-NOS and ROS as a result of bacterial infection, trauma, excess exposure to oxygen and noxious gases. These mediators induce endothelial and epithelial injury in lung, vascular leakage, edema, and vasodilatation, subsequently causing the development of ALI and ARDS (Densmore, Signorino et al. 2006). IL-10 primarily produced by T-helper 2 cells, B cells, monocytes, macrophages and keratinocytes is known to reduce the synthesis of pro-inflammatory cytokines and terminate inflammatory responses (Moore, Rousset et al. 1991). Low levels of IL-10 are found in patients with transfusion-related ALI (Kapur, Kim et al. 2017). Importantly, treatment with IL-10 alleviated lung injury induced by ischemia-reperfusion, lipopolysaccharide (LPS) (Bi, Wang et al. 2008), bleomycin and ozone; absence of endogenous IL-10 enhanced ALI induced by carrageenan. In addition to this, it was also reported that, pre-incubation of cultured fetal rat alveolar type II cells with recombinant IL-10 prior to 65% hyperoxia exposure decreased cellular necrosis and increased cell proliferation (Lee and Kim 2011). As reported by the present inventors (Bhandari 2008), and others (Li, Zhang et al. 2015), exogenous IL-10 treatment alleviated hyperoxia-induced ALI in mice, possibly by regulating neutrophil recruitment and the subsequent generation of cytokines, NO and matrix metalloproteinases.

CD163, a transmembrane scavenger receptor found on the surface of macrophages. Cell surface expression of CD163 on alveolar macrophages is reduced in human subjects with asthma (Dai, Yao et al. 2016) and BPD (Kwon, Kim et al. 2019), which suggests that CD163 may participate in the regulation of airway inflammatory responses in the lung. CD163 is released in the circulation in its soluble form, sCD163, via cleavage of the extracellular domain by matrix metalloproteases (MMPs) following oxidative stress (Timmermann and Hogger 2005) or via activation of TLR4 after inflammatory stimuli (Weaver, Pioli et al. 2007, Zhi, Gao et al. 2017) and used as a biomarker for injury/inflammation. sCD163 protects monocytes from hyperactivation during bacterial infections by dampening the secretion of the proinflammatory cytokines TNF-α, IL-1β, IL-6 and IL-8 (Kneidl, Loffler et al. 2012) and endocytosing inflammatory neutrophils, and excess hemoglobin/haptoglobin complexes, so possesses phagocytic activity in clearing cell debris.

### SUMMARY OF THE INVENTION

The present invention overcomes the drawbacks of the prior art by providing small molecular weight, water soluble, oligosaccharides compounds (1, 4, 22 and 26) with binding affinity for both immune receptors TLR4 and CD163 for the treatment of inflammation related disorders. The net effect of binding of these compounds of invention to both TLR4 and CD163 receptors was an increase in percentage of intermediate macrophages population composed of both M1 and M2 phenotypes that renders immune cell balance. In some embodiments the inflammation may be caused by mechanical ventilation or gram-negative bacterial or polymicrobial infections. In some embodiments the compounds find use in the treatment of bacterial lung infection induced pneumonia, chronic lung injury induced bronchopulmonary dysplasia in pre-term infants, sepsis and severe sepsis, SIRS and septic shock.

In some embodiments the compounds find use for treating inflammatory disorders, such as age-related macular degeneration (AMD) and retinopathy of prematurity pathogenesis. Such inflammatory conditions include, but are not limited to, ocular inflammatory diseases and choroidal neovascularization.

Compounds of the present invention have been unexpectedly found to possess superior anti-inflammatory activity in inhibiting inflammatory biomarkers such as TNF-α, IL-1β and IL-6 in LPS induced human mononuclear cell assay and producing anti-inflammatory cytokine IL-10. Compounds of present invention protected mice from both lethal gram-negative sepsis against Escherichia coli, polymicrobial sepsis in a cecal ligation and puncture model and P. aeruginosa induced lung pneumonia. Accordingly, the present disclosure provides methods of inhibiting inflammatory biomarkers such as, but not limited to, TNF-α, IL-1β and IL-6 in LPS induced human mononuclear cell assay by administering the compounds disclosed herein to a patient in need thereof. In addition, the present disclosure provides methods of protecting mice from lethal gram-negative sepsis against Escherichia coli and Pseudomonas aeruginosa by administering the compounds disclosed herein to a patient in need thereof. Compounds of the present invention have also been unexpectedly found to possess broad spectrum antimicrobial activity against both gram positive (methicillin susceptible Staphylococcus aureus and methicillin resistant Staphylococcus aureus), gram negative (E. coli, P. aeruginosa, A. baumannii, K. pneumonia) bacteria as well as fungus (C. albicans) mostly found in burn and septic wounds. Accordingly, the present disclosure provides methods of treating infection caused by both gram positive (methicillin susceptible Staphylococcus aureus and methicillin resistant Staphylococcus aureus), gram negative (E. coli, P. aeruginosa, A. baumannii, K. pneumonia) bacteria as well as fungus (C. albicans) by administering compounds disclosed herein to a patient in need thereof. Compounds of the present invention have also been unexpectedly found to inhibit and eradicate biofilm formed by S. aureus. Accordingly, the present disclosure provides methods of inhibiting or eradicating biofilm formed by a microorganism, such as but not limited to S. aureus by contacting a surface with compounds disclosed herein. Compounds of present invention also demonstrated superior in vivo efficacy in protecting mice against cecal ligation and puncture (CLP) induced death and organ dysfunction.

Compounds of the present invention have also been unexpectedly found to possess superior activity against HMGB1 induced inflammation in mouse macrophages and reduced VEGF expression in retinal pigmental epithelial cells (ARPE-19) and daily intraperitoneal injection of the compound was able to reduce the average size of choroidal neovascularization (CNV) lesions to about 60% of those in control mice treated with vehicle only. Accordingly, the disclosure provides methods of protecting a subject in need thereof from infection or disorders associated with infection by treating said subject with a compound as disclosed herein.

As embodied and broadly described herein, an aspect of the present disclosure relates to a pharmaceutical composition comprising a compound according to Formula (I), or a pharmaceutically acceptable salt thereof:

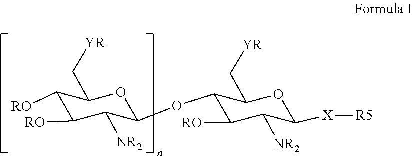

R=H, C(O)R₁, alkyl, benzyl, substituted benzyl;
R₁=CH₃, alkyl, piperidine nitroxyl, or biotin;
R₂=H, C(O)R¹, C(S)NR¹ or aceloxy alkyl carbamate of the following formula:
R₂=C(O)OCHR₃OC(O)OR₄, piperidine nitroxyl, or fluorescein isothiocyanate (FITC);
R₃=H, CH₃, C₂H₅, isopropyl;
R₄=substituted alkyl group;
X=H, O, NH, or S and is linked to the anomeric carbon via R stereochemistry (beta anomer) at a pH of 6.5-7.4;

### Y=O, NH or S;

R₅=aryl, substituted aryl, heteroaryl, substituted heteroaryl, cycloalkyls and substituted cycloalkyl, piperidine nitroxyl, piperidine N-hydroxylamine,

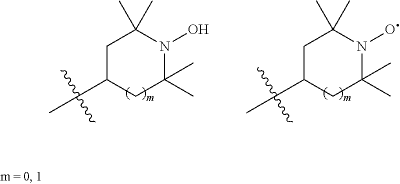

wherein the substituted aryl groups are defined as below

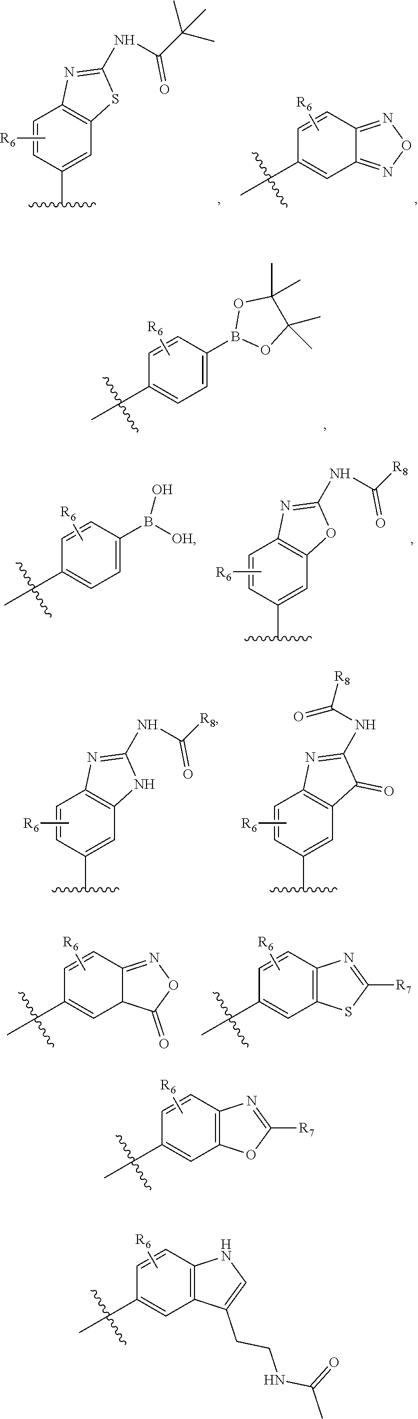

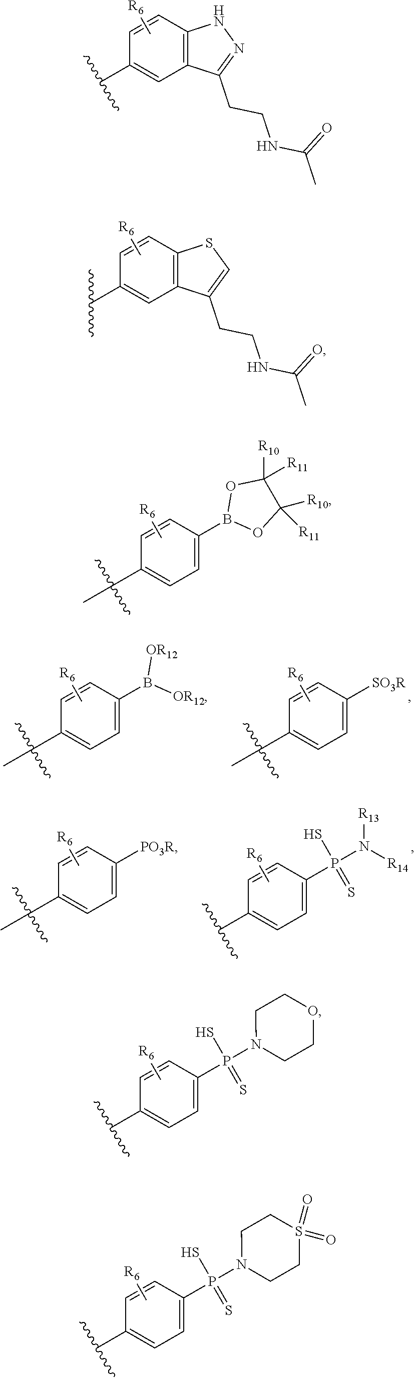

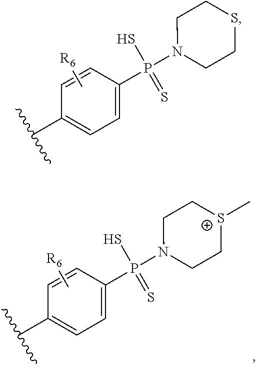

R₆=H, NR, OR₃, SR₃, Cl, Br, F, I, NO₂, CO₂H, CO₂R₃, R₇ to R₁₁ are selected from: H, acyl, alkyl, substituted alkyl, alkenyl, alkynyl, alkoxy, aryloxy, alkoxycarbonyl, amido, amino, carbonate, carbamate, carbonyl, ester, halo, hydroxy, phosphate, phosphonate, phosphinate, phosphine oxide, urea, cycloalkyl, heterocycloalkyl, aryl, and heteroaryl, C(O)R¹; and n=0-7.

In one aspect, the composition is formulated a sterile, injectable aqueous or oleaginous suspension. In another aspect, the composition is formulated as a sterile topical gel, ointment or aqueous spray. In another aspect, the composition further comprises an anti-inflammatory agent, an antimicrobial agent, or both. In another aspect, the compound of Formula (I) is further defined as:

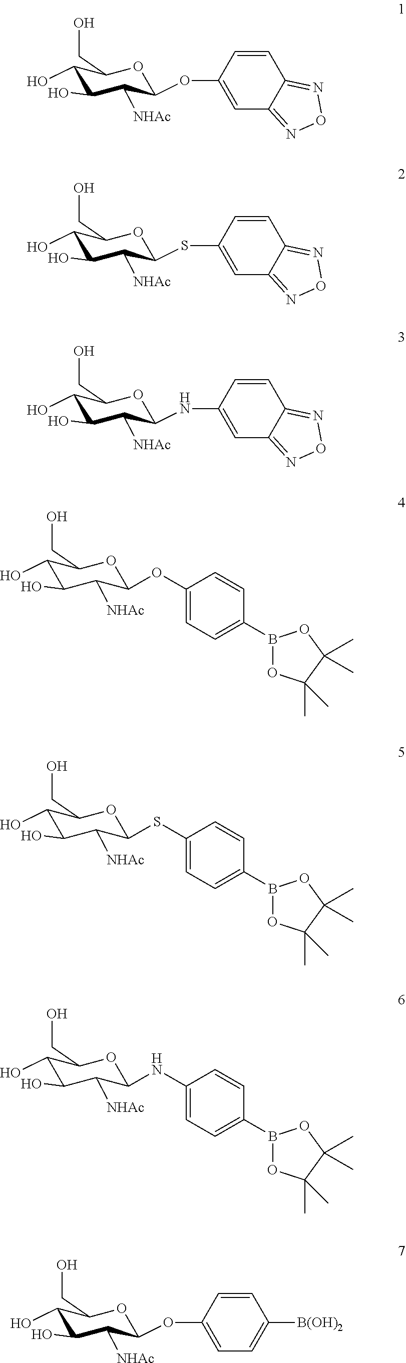

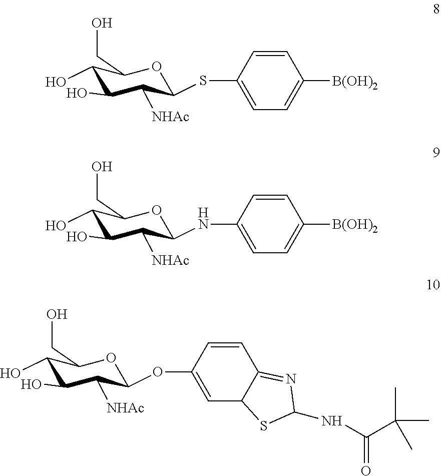

In another aspect, the compound of Formula (I) is further defined as: n=0, X=NH, Y=N, R=Biotin, R₂=C(O)CH₃, R₅=aryl, substituted aryl or substituted heteroaryl

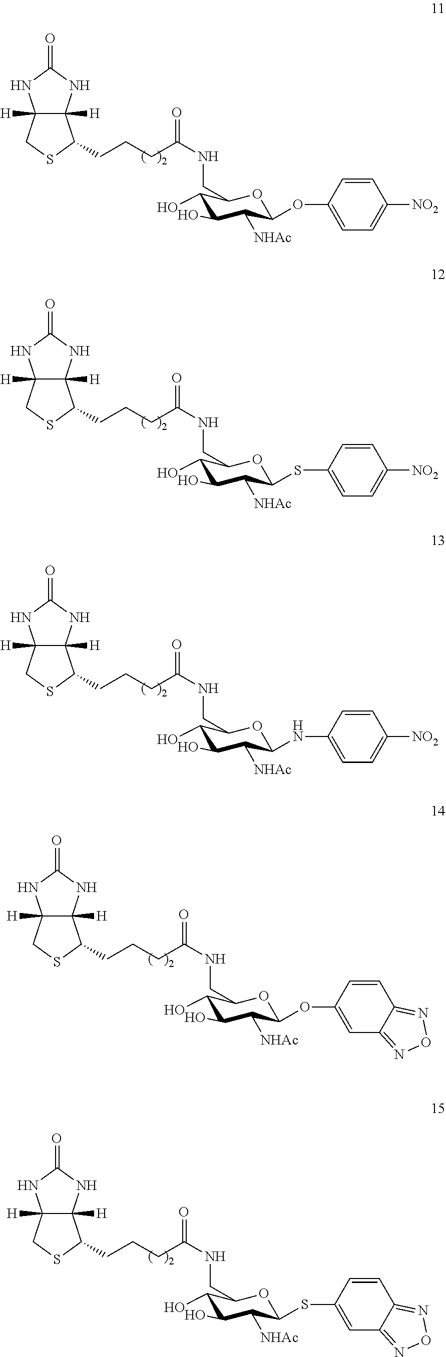

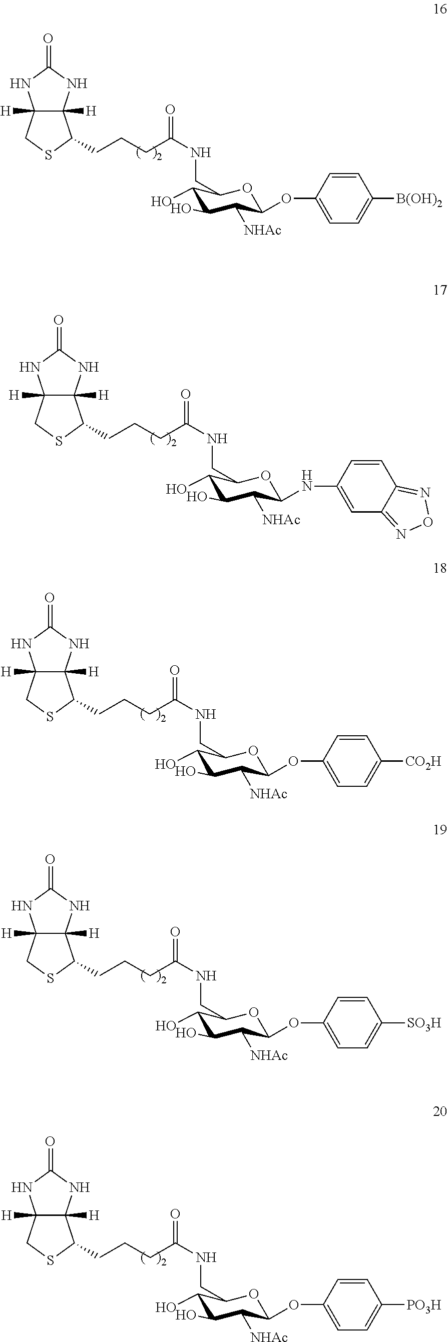

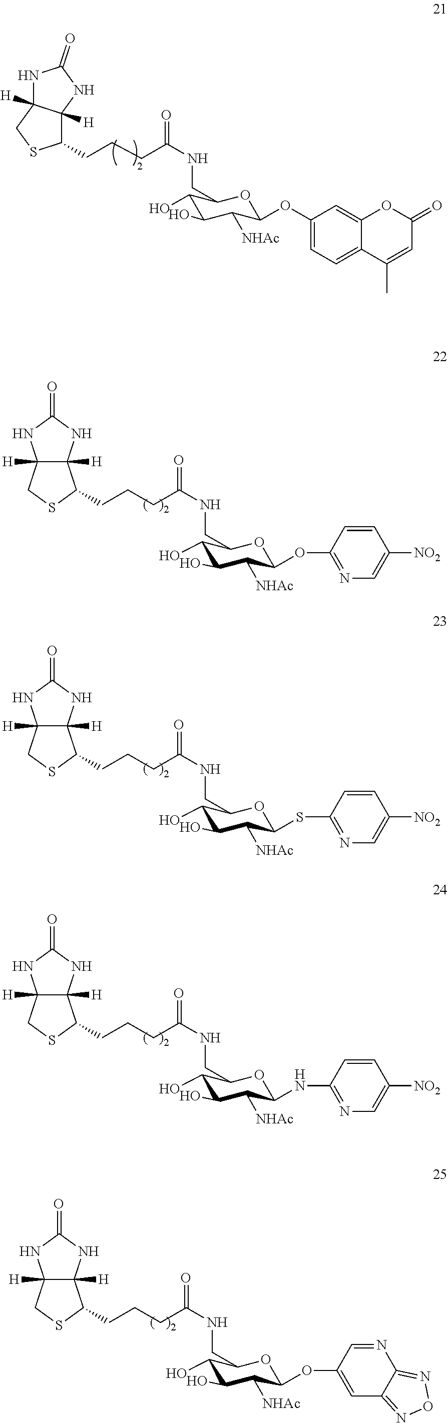

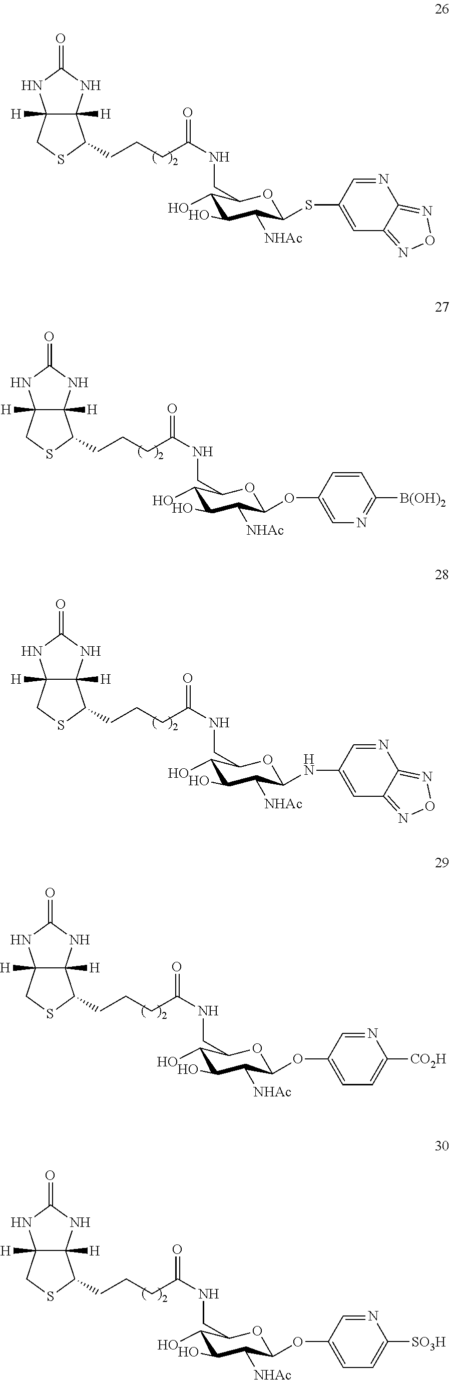

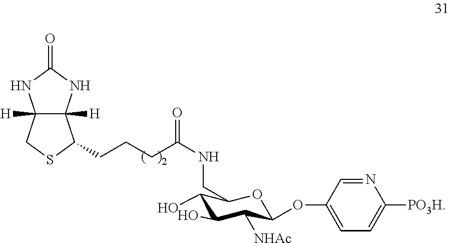

In another aspect, the compound of Formula (I) is further defined as: n=0, X=NH, Y=N, R=Biotin, R₂=C(O)CH₃, R₅=cycloalkyl or heterocycloalkyl

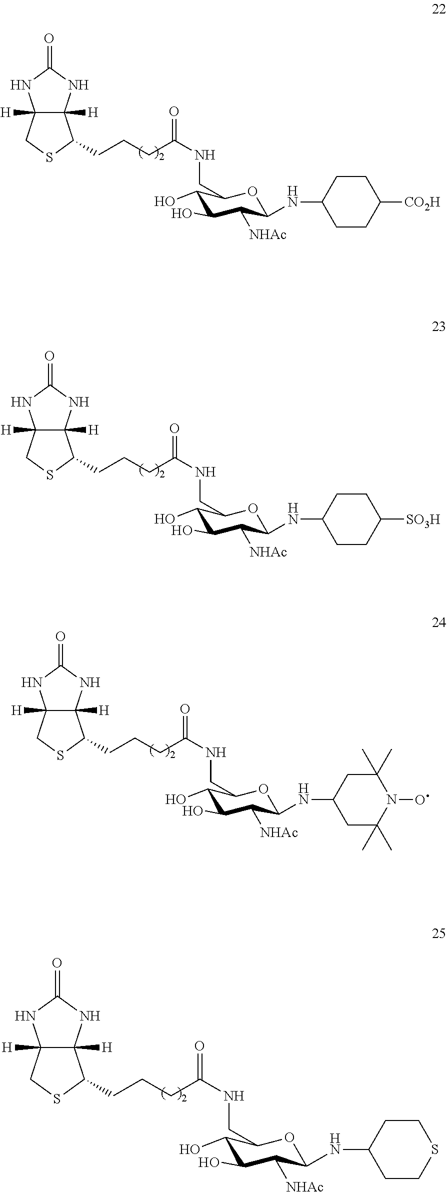

In another aspect, the compound of Formula (I) is further defined as: n=0, X=O, Y=O, R=H, R₂=FITC, R₅=aryl or substituted aryl

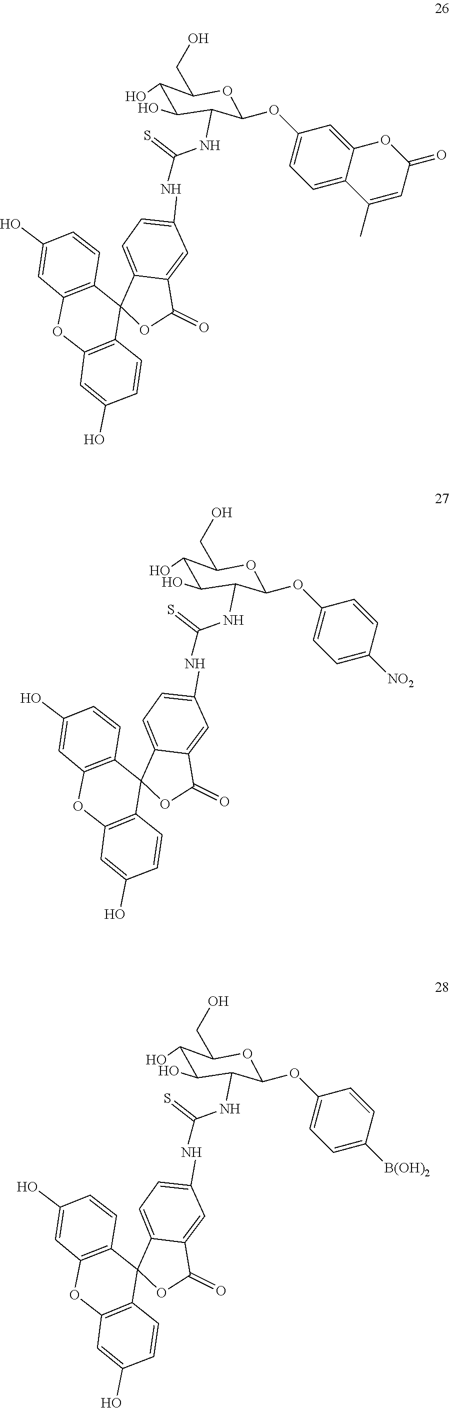 [SMILES: Cc1cc(=O)oc2cc(OC3OC(CO)C(O)C(O)C3NC(=S)Nc3ccc4c(c3)C(=O)OC43c4ccc(O)cc4Oc4cc(O)ccc43)ccc12 ; O=C1OC2(c3ccc(O)cc3Oc3cc(O)ccc32)c2ccc(NC(=S)NC3C(Oc4ccc([N+](=O)[O-])cc4)OC(CO)C(O)C3O)cc21 ; O=C1OC2(c3ccc(O)cc3Oc3cc(O)ccc32)c2ccc(NC(=S)NC3C(Oc4ccc(B(O)O)cc4)OC(CO)C(O)C3O)cc21]

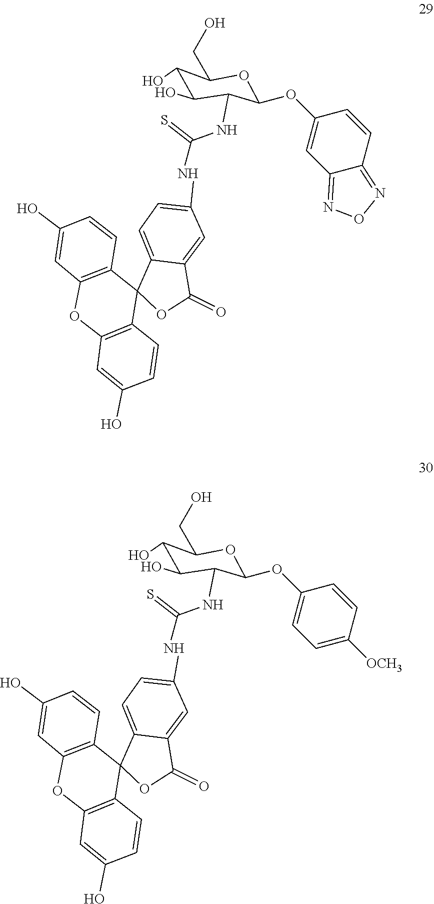 [SMILES: O=C1OC2(c3ccc(O)cc3Oc3cc(O)ccc32)c2ccc(NC(=S)NC3C(Oc4ccc5nonc5c4)OC(CO)C(O)C3O)cc21 ; COc1ccc(OC2OC(CO)C(O)C(O)C2NC(=S)Nc2ccc3c(c2)C(=O)OC32c3ccc(O)cc3Oc3cc(O)ccc32)cc1]

In another aspect, the compound of Formula (I) is further defined as: n=2-7, X=OH, Y=O, R=H, R₂=H, C(O)CH₃, FITC, or piperidine nitroxyl, R₅=H, cyclohexyl, heterocycloakyl

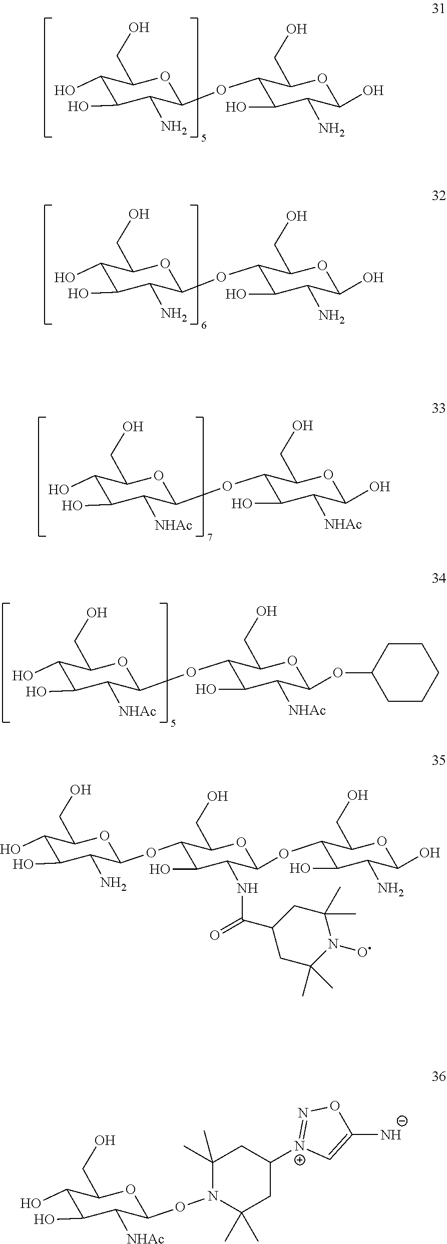

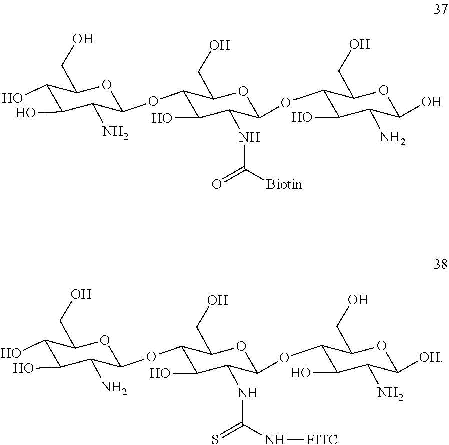

As embodied and broadly described herein, an aspect of the present disclosure relates to a method of treating at least one of sepsis, septicemia, septic shock, peritonitis, skin and soft tissue infections, ocular infection, ocular inflammation, ocular angiogenesis, rheumatoid arthritis (RA), atherosclerosis, inflammatory bowel diseases (IBD), necrotizing enterocolitis, asthma, chronic obstructive pulmonary disease, acute respiratory distress syndrome, acute lung injury, bacterial and viral pneumonia, chronic lung injury, acute kidney injury, chronic kidney injury, fibrosis, fever syndromes, cachexia, psoriasis, autoimmune diseases, cardiac diseases, retinoblastoma, cancer, disorder associated with inflammation, immunomodulation or microbial infections which comprises administering to a subject in need thereof a therapeutically effective amount of a pharmaceutical composition of Formula (I), or a pharmaceutically acceptable salt thereof, whereby the subject is treated. In another aspect, the step of administering comprises providing a pharmaceutical compound comprising about 5.0 mg to about 100 mg of a compound according to Formula (I), or a pharmaceutically acceptable salt thereof to a patient in need thereof, whereby the patient is treated. In another aspect, the step of administering comprises administering the pharmaceutical composition comprising about 10.0 mg to about 1000 mg of a compound according to Formula (I), or a pharmaceutically acceptable salt thereof to a patient in need thereof, whereby the patient is treated.

As embodied and broadly described herein, an aspect of the present disclosure relates to a composition comprising an effective amount of a compound according to Formula (I), or a pharmaceutically acceptable salt thereof:

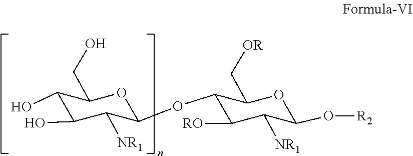

where:
n=0-1
R=benzyl, substituted benzyl
R₁=COCH₃, N-dimethylmaleimide
R₂=cyclohexyl, p-nitro phenyl, piperidine nitroxy, piperidine-N-hydroxyl, p-methoxy phenyl, and a pharmaceutically acceptable excipient. In one aspect, the composition is formulated a sterile, injectable aqueous or oleaginous suspension. In another aspect, the composition is formulated as a sterile topical ocular solution. In another aspect, the compound is selected from at least one of:

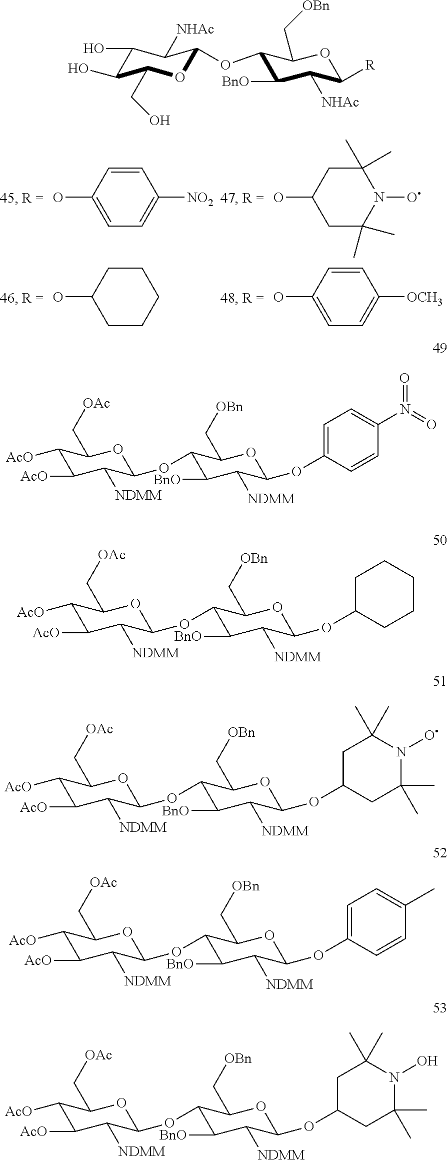

As embodied and broadly described herein, an aspect of the present disclosure relates to a method of treating ocular angiogenesis, ocular inflammation which comprises administering to a subject in need thereof a therapeutically effective amount of a pharmaceutical composition according to claim Error! Reference source not found, or a pharmaceutically acceptable salt thereof, whereby said subject is treated. In one aspect, the compounds are selected from at least one of compounds 38 to 44:

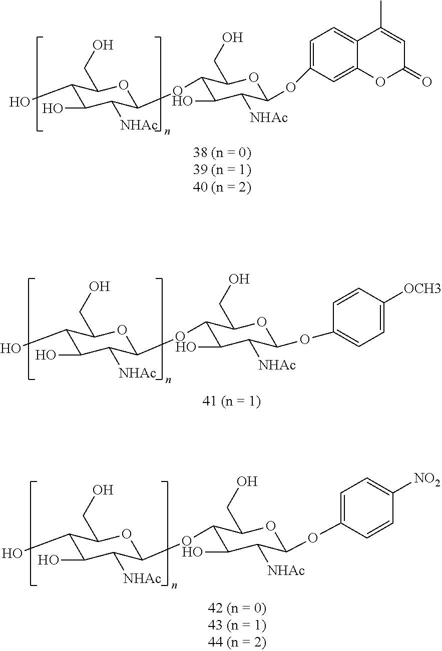

Thus, in some embodiments, the present invention includes compounds for the treatment of sepsis, septicemia, septic shock, peritonitis, skin and soft tissue infections, ocular infection, ocular inflammation, ocular angiogenesis, rheumatoid arthritis (RA), atherosclerosis, inflammatory bowel diseases (IBD), necrotizing enterocolitis, asthma, chronic obstructive pulmonary disease, acute respiratory distress syndrome, acute lung injury, bacterial and viral pneumonia, chronic lung injury, acute kidney injury, chronic kidney injury, fibrosis, fever syndromes, cachexia, psoriasis, autoimmune diseases, cardiac diseases, retinoblastoma, cancer and/or any disorder associated with inflammation, immunomodulation and microbial infection.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. However, figures provided herein are intended to help illustrate the invention or assist with developing an understanding of the invention, and are not intended to be definitions of the invention's scope.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a graph showing a graph with anti-pseudomonal activity of compounds 1 and 4 in mouse lungs by decreasing bacterial CFUs.

FIG. 2 is a graph showing the phagocytosis and intracellular bacteria killing activity of compound 4 in human monocyte derived macrophages model.

FIG. 3 is a table showing the synergistic and additive activity of bacteria killing activity by compound 4 in combination with standard of care antibiotics.

FIG. 4 showing prevention of chronic lung injury by compounds 4 in juvenile mouse BPD model after intraperitonial dosing.

FIG. 5 showing the clean genotoxicity profile of compounds 1, 4, 34 and 42 in bacteria mutation and in vitro micronucleus studies.

FIG. 6 showing affinity for hERG channel binding of compounds 1, 34 and 42 in HEK293 cell assay.

FIG. 7 showing no off targets activities of compounds 4 and 42 in Eurofin's 87 receptor, enzyme, ion channel assay.

FIG. 8 showing no off targets activities of compounds 4 and 42 in Eurofin's 87 receptor, enzyme, ion channel assay.

FIGS. 9A and 9B showing pharmacokinetic profile of compounds 1 in adult rats after intravenous dosing.

FIG. 10 is a graph showing binding of compound 11 to mouse spleen derived monocytes in a dose dependent manner.

FIG. 11 is a graph showing binding of compound 11 to the TLR4 present in mouse spleen derived monocytes in a dose dependent manner.

FIG. 12 is a graph showing binding of compound 11 to the CD163 receptor present in mouse spleen derived monocytes in a dose dependent manner.

FIGS. 13A and 13B are micrographs showing no treatment and bioavailability of compound 26 in mouse lung after intranasal administration of the nanosuspension formulation.

FIG. 14 is a graph showing the results of an evaluation of the TLR4 modulator compound 31 of the present invention in a mouse endotoxemia sepsis model and illustrates that compound 31 protected mice from lethal gram-negative sepsis against E. coli at a dose of 10 mg/kg.

FIGS. 15A to 15D are graphs showing the results of an evaluation of the TLR4/CD163 modulators AVR-25 (compound 34) AVR-45 (compound 39), AVR-48 (compound 42) and compound 43 of the present invention in a cecal ligation and puncture (CLP) model and illustrates that all the compounds protected mice from CLP induced polymicrobial sepsis and death.

FIGS. 16A to 16C are the histopathology score of major organs which demonstrates that, on treatment of compounds 34 and 44, the compounds of present invention reversed the major pathological changes and tissues resembled to sham group.

FIG. 17 demonstrated that compound 34 of present invention effectively down regulates the inflammatory cytokines in vivo in CLP mice.

FIG. 18 demonstrates that an evaluation of the TLR4 modulating compound 32 of the present invention in a lased induced CNV mouse model for wet AMD. Compound 32 decreased the choroidal neovascularization ˜60% as compared to the positive control.

FIG. 19 demonstrates that compounds of present invention (31, and 32) decreased HMGB1 induced VEGF production in ARPE-19 cell.

FIG. 20 demonstrates that compounds of present invention inhibit HMGB1 induced production of inflammatory mediator (TNF-α) in mouse bone marrow derived macrophages.

FIG. 21 demonstrates that compounds of present invention inhibit HMGB1 induced production of inflammatory mediators (TNF-α, i-NOS) and upregulate M2 biomarker CXCR4 in mouse macrophages.

FIGS. 22A, 22B, 22C and 22D demonstrate that compounds of present invention inhibited LPS induced production of inflammatory mediators in human blood cells.

FIGS. 23A, 23B, 23C, 23D and 23E demonstrate that compounds of present invention produce anti-inflammatory cytokine IL-10 in human blood cells.

FIG. 24 demonstrates that compounds of present invention decrease LPS induced sCD163 level in human blood cells.

FIG. 25 demonstrates that compounds of present invention have broad-spectrum antimicrobial activity.

FIG. 26 demonstrates that compounds of present invention inhibited biofilm formation.

FIG. 27 demonstrates that the broad-spectrum antimicrobial activity of the compounds of present invention is via disruption of cell membrane.

FIGS. 28A and 28B demonstrate that compounds of present invention do not bind to the plasma serum protein.

FIG. 29 shows the synthetic scheme for preparing compound 4 and analogs.

FIG. 30 shows the synthetic scheme for preparing compound 42.

FIG. 31 provides the synthetic scheme for preparing compound 11.

FIG. 32 provides the synthetic scheme for preparing compound 35 and 37.

### DETAILED DESCRIPTION OF THE INVENTION

In sepsis caused by gram-negative bacteria, lipopolysachharide (LPS) activates the immune system through signaling receptor Toll Like Receptor 4 (TLR4) to initiate the process for production of inflammatory cytokines (TNF-α, IL-1β, IL-6, ROS) responsible for hyper-inflammation. Thus, some investigators are seeking to develop antagonists that block either activation through TLRs or downstream signaling pathways that inhibit the storm of inflammatory molecules. However, none of the TLR4 inhibitor has the potential to balance the immune response and not suppress the immunity which is critical for development of secondary infection during sepsis.

The multifaceted pathological complexity of intra-abdominal sepsis or ARDS involving infection, inflammation, immune suppression is difficult to treat effectively only with antibiotics. Sepsis develops when the inflammatory response to infection rises to such a level that physiologic alterations within the host occur. The bacterial load may be of such magnitude (or the bacteria may be highly virulent) that an exaggerated inflammatory response appropriately matches the powerful bacterial stimulus. This concomitant “collateral damage” to normal cellular and tissue function cannot be adequately compensated for with antibiotics alone in the septic patient. In this context, an anti-inflammatory intervention such as corticosteroids may reduce the toxic effects of the inflammatory response but may also compromise effective host protection from the infection, especially in geriatric and immunocompromised patients.

The compounds of present innovation are small molecules that can synergistically inhibit inflammation, increase phagocytosis of bacteria, decrease microbial infection. The compounds of current invention decrease the bacterial CFUs via binding to TLR4 and CD163 receptors activating the phagocytic pathway and decreasing inflammation with therapeutic potential to treat sepsis, septicemia and septic shock, ARDS, bacterial and viral pneumonia without compromising the immunity. Thus, the compounds and compositions of the present invention can be used to treat at least one of sepsis, septicemia, septic shock, peritonitis, skin and soft tissue infections, ocular infection, ocular inflammation, ocular angiogenesis, rheumatoid arthritis (RA), atherosclerosis, inflammatory bowel diseases (IBD), necrotizing enterocolitis, asthma, chronic obstructive pulmonary disease, acute respiratory distress syndrome, acute lung injury, bacterial and viral pneumonia, chronic lung injury, acute kidney injury, chronic kidney injury, fibrosis, fever syndromes, cachexia, psoriasis, autoimmune diseases, cardiac diseases, retinoblastoma, cancer, disorder associated with inflammation, immunomodulation or microbial infections.

TLR4 signaling is also essential for survival in acute lung injury induced by virulent P. aeruginosa type III secretory toxins found in multi-drug resistance (MDR) strains. Thus, it is required to maintain a balance between inflammation and resolution The compounds of present invention bind to TLR4 in such a way that there is selective activation of the target cell so that there is increase in expression of the important anti-inflammatory cytokine IL-10. The anti-inflammatory cytokine IL-10 is an important endogenous regulator of chemokine expression in acute lung inflammation.

Posterior segment neovascular ocular diseases, as exemplified by proliferative diabetic retinopathy (PDR), exudative age-related macular degeneration (AMD) and retinopathy of prematurity (ROP), are a growing and huge health threat which require new effective therapies. Retinal neovascularization associated with PDR is the leading cause of blindness in working age adults. Choroidal neovascularization (CNV) is responsible for 200,000 new cases of exudative AMD each year in the US rendering this neovascular pathology the leading cause of legal blindness in non-third world nations. The projected number of people with AMD in 2020 is 196 million, increasing to 288 million in 2040 [Wong et al, The Lancet Global Health 2014, 2, e106-e116]. Pathological angiogenesis associated with ROP is the major cause of blindness in children under the age of seven [Harrell et al, Neonatal Network 2007, 26, 371-378].

Multiple lines of evidence suggest that Toll Like Receptor (TLR4) signaling may be associated with pathologic changes in retinal diseases [Cho et al, Investigative Ophthalmology & Visual Science 2009, 50, 5614-5618], including AMD eyes by oxidized lipids, lipofuscin and by drusen components. Once activated, TLR4 could contribute to the pathogenesis of AMD by multiple mechanisms such as release of TNF-α, interleukin-1β, and other pro-inflammatory mediators. TLR4 activation suppresses Wnt signaling, leading to reduced growth factor expression, secretion, and increased photoreceptor death in response to oxidative stress as well as can also lead to oxidative damage of photoreceptor outer segments. TLR4 has a direct effect on several inflammation-related signaling pathways including MAPK, NFκ-β and Jak1/Stat1 and shown to mediate neuronal toxicity through caspase-3, neuronal iNOS and ERK1/2, JNK1/2 and p38. Interestingly, TLR4-mediated microglial activation by endogenous photoreceptor proteins in retinal inflammation can aggravate retinal cell death. Finally, release of high-mobility group box-1 in ischemic neural tissue has been shown to initiate TLR4-dependent responses that contribute to retinal neovascularization [He et al, Arteriosclerosis, Thrombosis, and Vascular Biology. 2013; 33:330-338].

Accordingly, there exists a need for more effective treatments for inflammation and in particular for both dry and wet AMD pathogenesis. The compounds and methods described herein, therefore demonstrated that inhibition of TLR4 activity is of therapeutic value in AMD and other retinal diseases. The compounds of present innovation are small molecules that can synergistically inhibit angiogenesis, inflammation and accelerate phagocytosis with therapeutic potential to treat AMD.

Prior to the present disclosure, however, it does not appear that there are any reports published on the use of chitohexaose (compound 31), chitohepatose (compound 32) and chitooctaose (compound 33) and derivatives thereof as TLR4 antagonist for inhibiting inflammation and angiogenesis for ocular indications such as dry/wet AMD, diabetic retinopathy, or any chronic ocular inflammation.

In one embodiment, the principles of the present disclosure provide a compound of Formula (I):

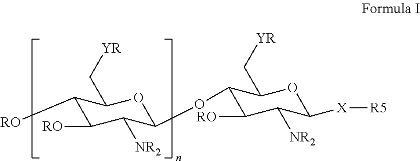

where:
R=H, C(O)R₁, alkyl, benzyl, substituted benzyl;
R₁=CH₃, alkyl, piperidine nitroxyl, or biotin;
R₂=H, C(O)R¹, C(S)NR¹ or aceloxy alkyl carbamate of the following formula:
R₂=C(O)OCHR₃OC(O)OR₄, piperidine nitroxyl, or fluorescein isothiocyanate (FITC);
R₃=H, CH₃, C₂H₅, isopropyl;
R₄=substituted alkyl group;
X=H, O, NH, or S and is linked to the anomeric carbon via R stereochemistry (beta anomer) at a pH of 6.5-7.4;
Y=O, NH or S;

R₅=aryl, substituted aryl, heteroaryl, substituted heteroaryl, cycloalkyls and substituted cycloalkyl, piperidine nitroxyl, piperidine N-hydroxylamine,

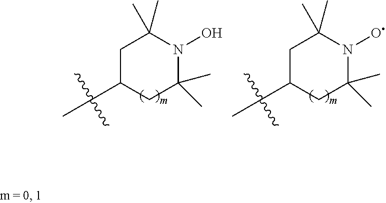

wherein the substituted aryl groups are defined as below

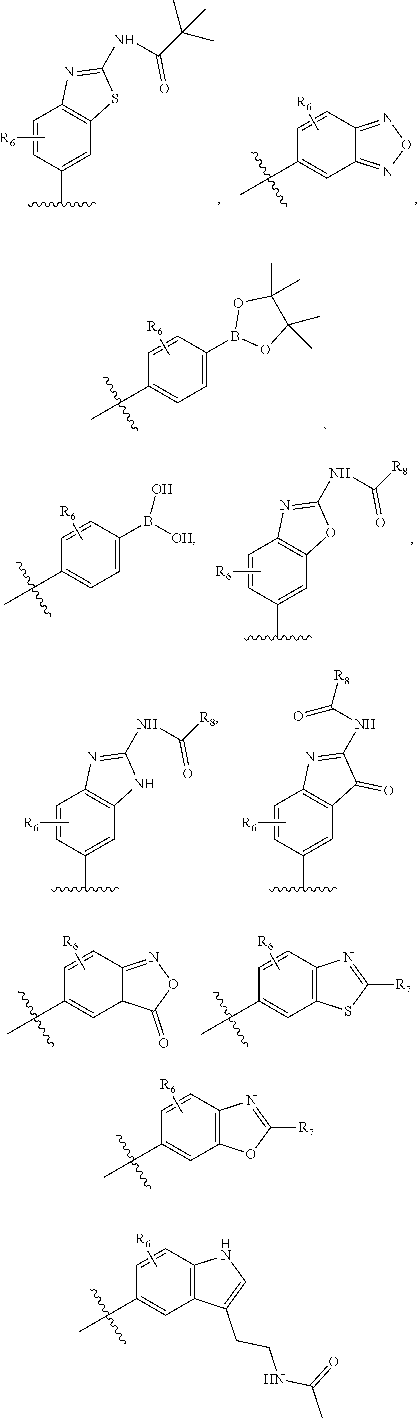

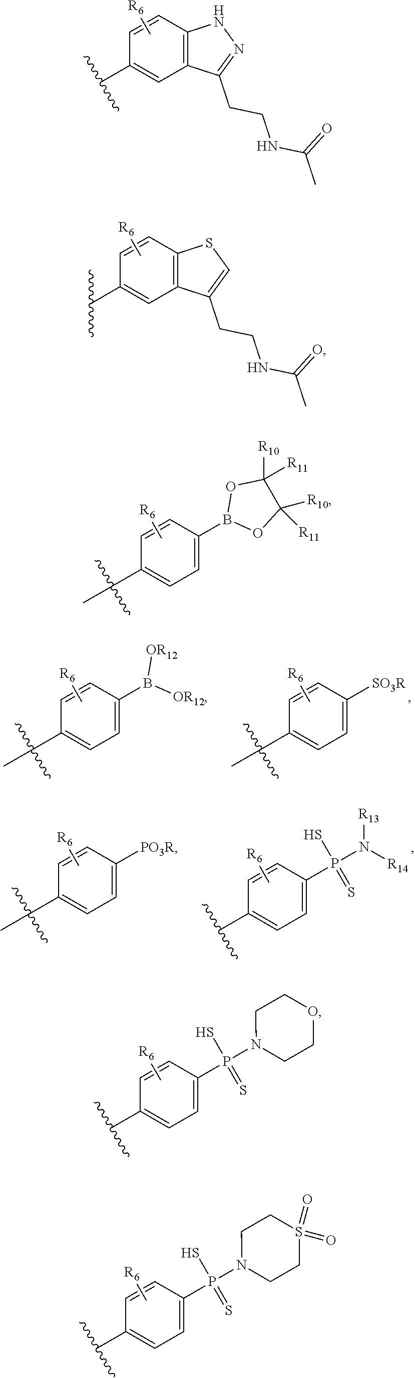

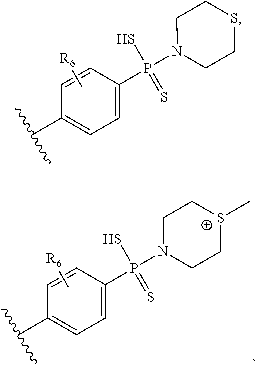

R₆=H, NR, OR₃, SR₃, Cl, Br, F, I, NO₂, CO₂H, CO₂R₃
R₇ to R₁₁ are selected from: H, acyl, alkyl, substituted alkyl, alkenyl, alkynyl, alkoxy, aryloxy, alkoxycarbonyl, amido, amino, carbonate, carbamate, carbonyl, ester, halo, hydroxy, phosphate, phosphonate, phosphinate, phosphine oxide, urea, cycloalkyl, heterocycloalkyl, aryl, and heteroaryl, C(O)R¹; and
n=0-7.

Additional compounds of the present disclosure include the following structures shown below where:

n=0, X=O, S or NH, Y=O, R=H, R₂=C(O)CH₃, R₅=substituted aryls, and substituted heteroaryl groups shown below

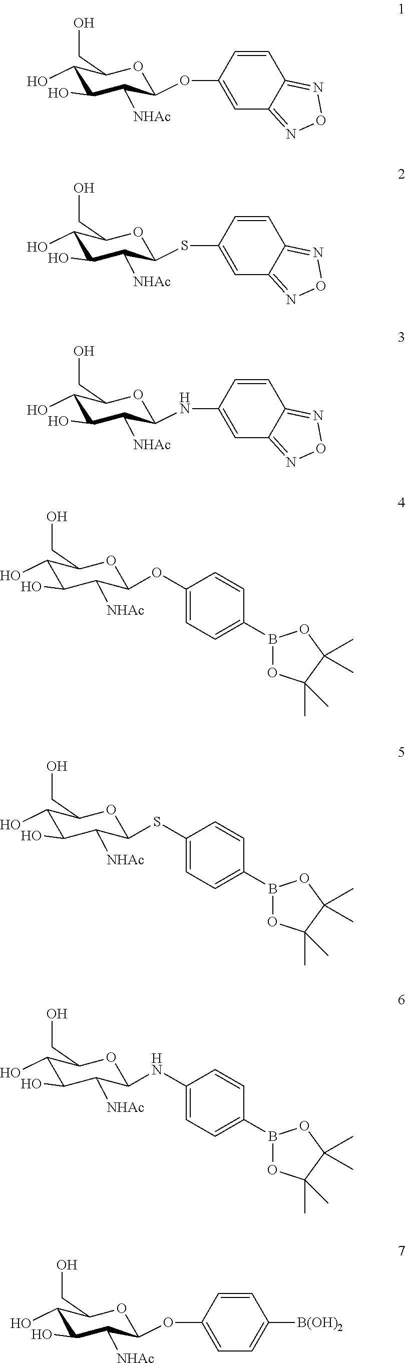

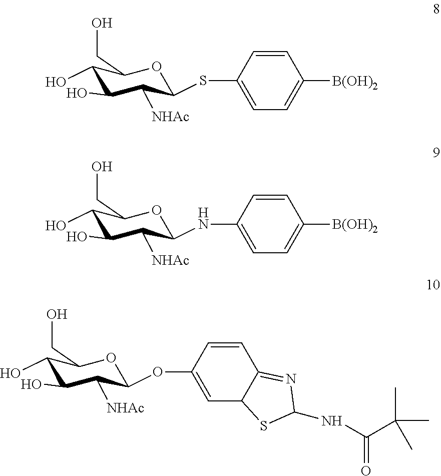

Additional compounds of the present disclosure include the following structures shown below where:

n=0, X=NH, Y=N, R=Biotin, R₂=C(O)CH₃, R₅=aryl, substituted aryl or substituted heteroaryl

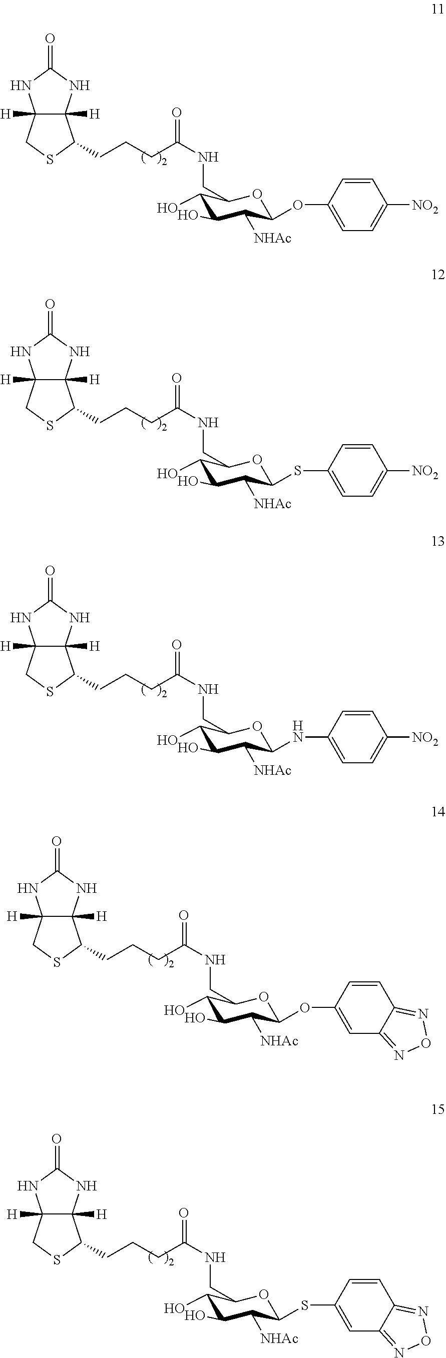

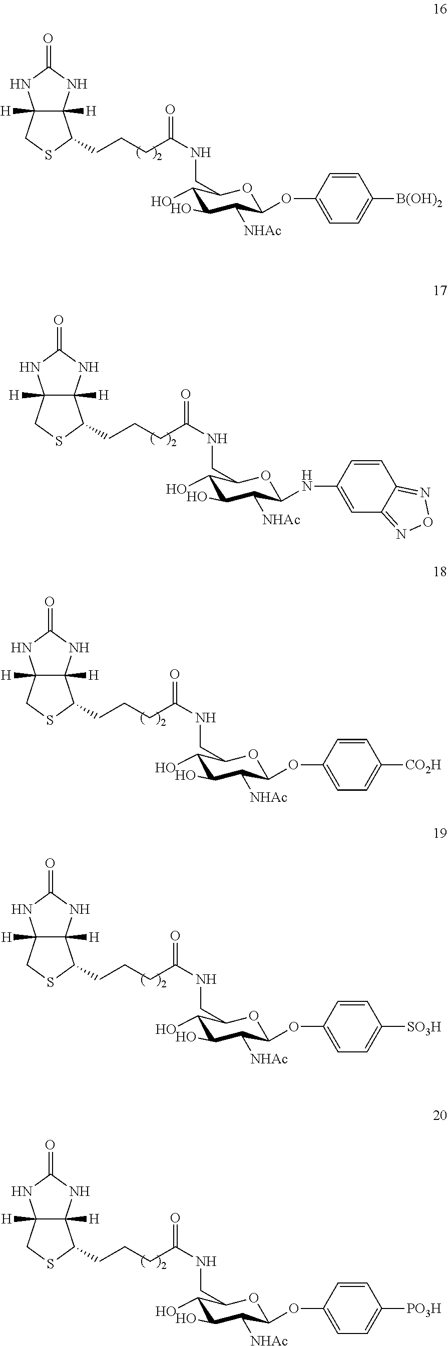

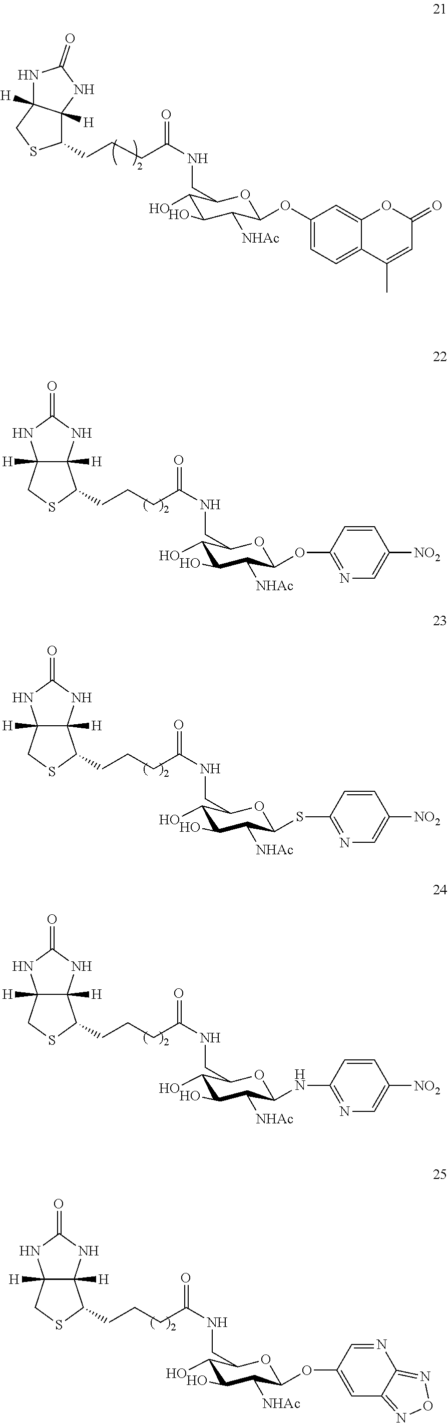

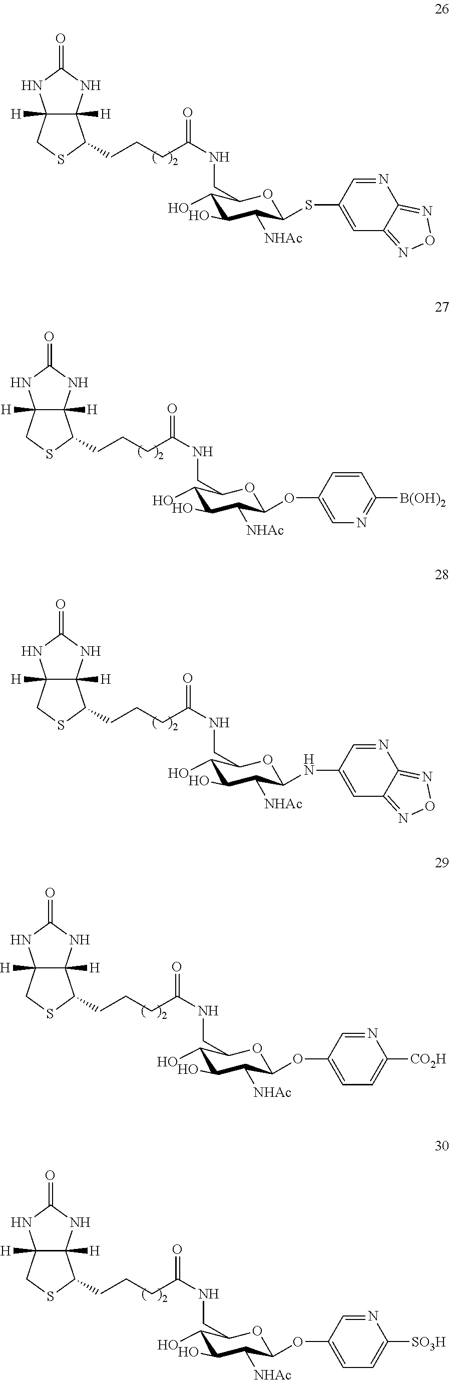

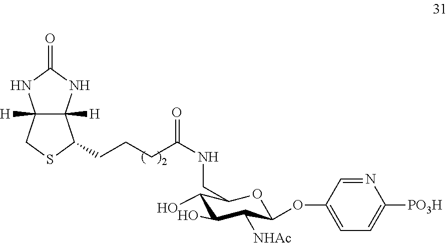

Additional compounds of the present disclosure include the following structures shown below where:

n=0, X=NH, Y=N, R=Biotin, R₂=C(O)CH₃, R₅=cycloalkyl or heterocycloalkyl

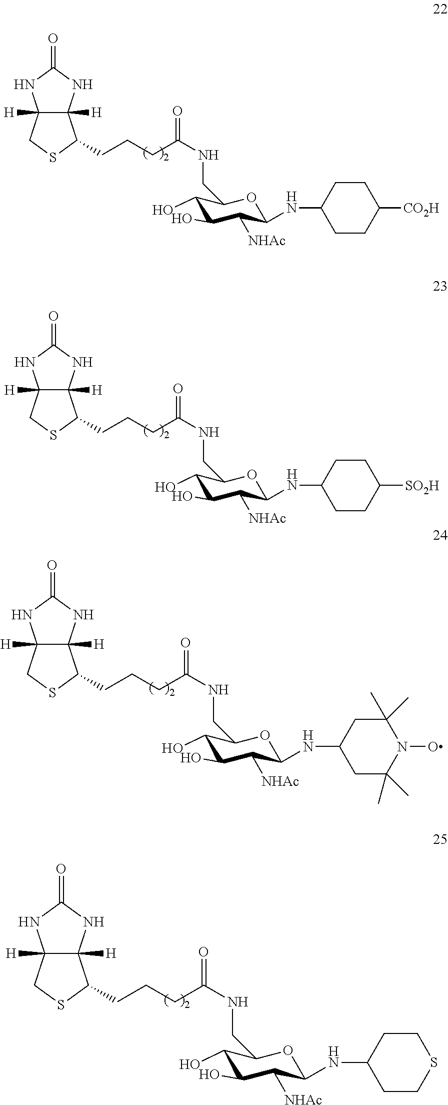

Additional compounds of the present disclosure include the following structures shown below where:

n=0, X=O, Y=O, R=H, R₂=FITC, R₅=aryl or substituted aryl

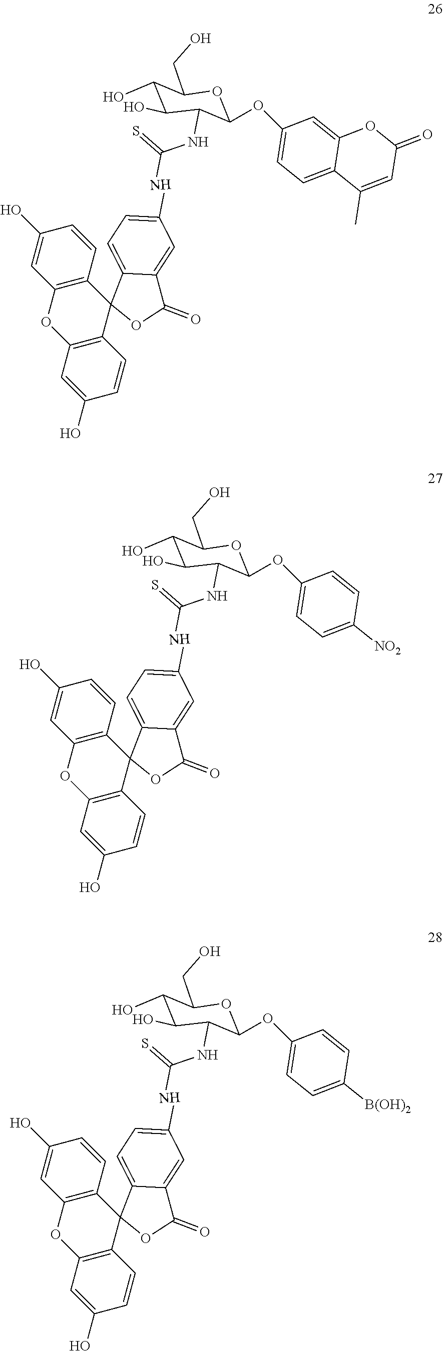 [SMILES: Cc1cc(=O)oc2cc(OC3OC(CO)C(O)C(O)C3NC(=S)Nc3ccc4c(c3)C(=O)OC43c4ccc(O)cc4Oc4cc(O)ccc43)ccc12 ; O=C1OC2(c3ccc(O)cc3Oc3cc(O)ccc32)c2ccc(NC(=S)NC3C(Oc4ccc([N+](=O)[O-])cc4)OC(CO)C(O)C3O)cc21 ; O=C1OC2(c3ccc(O)cc3Oc3cc(O)ccc32)c2ccc(NC(=S)NC3C(Oc4ccc(B(O)O)cc4)OC(CO)C(O)C3O)cc21]

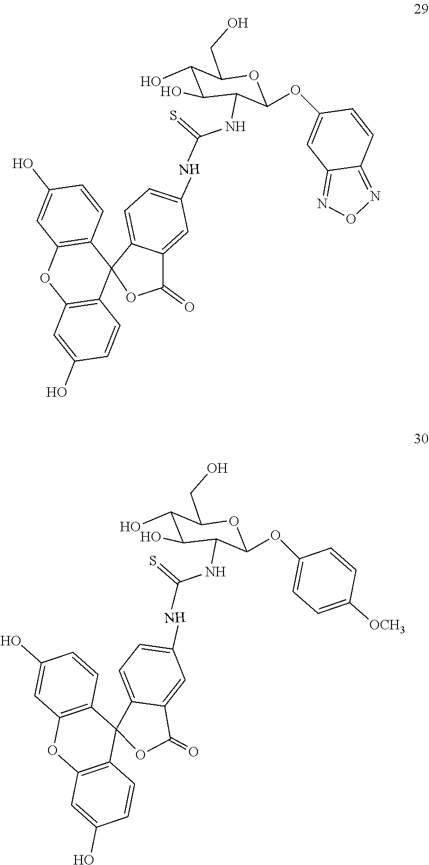 [SMILES: O=C1OC2(c3ccc(O)cc3Oc3cc(O)ccc32)c2ccc(NC(=S)NC3C(Oc4ccc5nonc5c4)OC(CO)C(O)C3O)cc21 ; COc1ccc(OC2OC(CO)C(O)C(O)C2NC(=S)Nc2ccc3c(c2)C(=O)OC32c3ccc(O)cc3Oc3cc(O)ccc32)cc1]

Additional compounds of the present disclosure include the following structures shown below where:

n=2-7, X=OH, Y=O, R=H, R₂=H, C(O)CH₃, FITC, or piperidine nitroxyl, R₅=H, cyclohexyl, heterocycloakyl

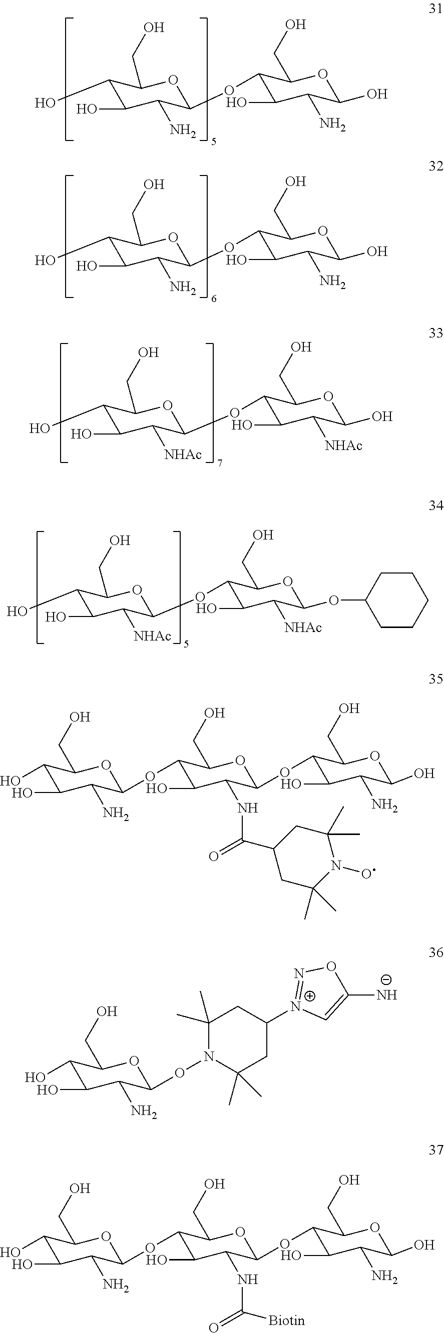

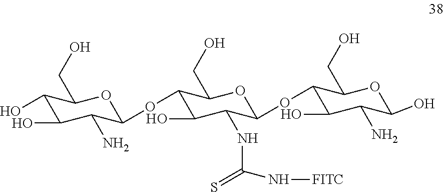

These compounds showed potent activity in inhibiting LPS and HMGB1 induced inflammation biomarkers (TNF-α, IL-1β and IL-6) in bone derived mouse macrophages as well as in human monocytes. Compounds 31 and 32 decrease the production of VEGF in ARPE-19 cells. Compound 32 showed significant decrease in CNV size in lased induced mice model for wet AMD.

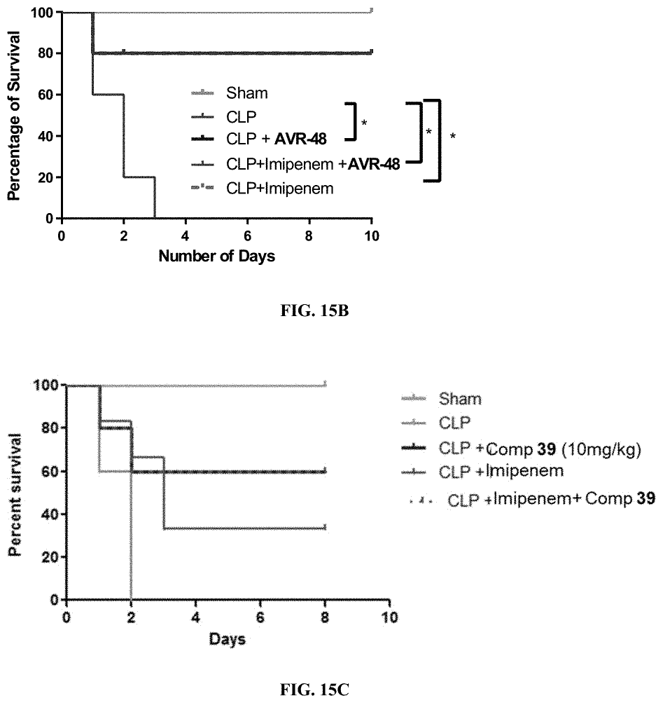

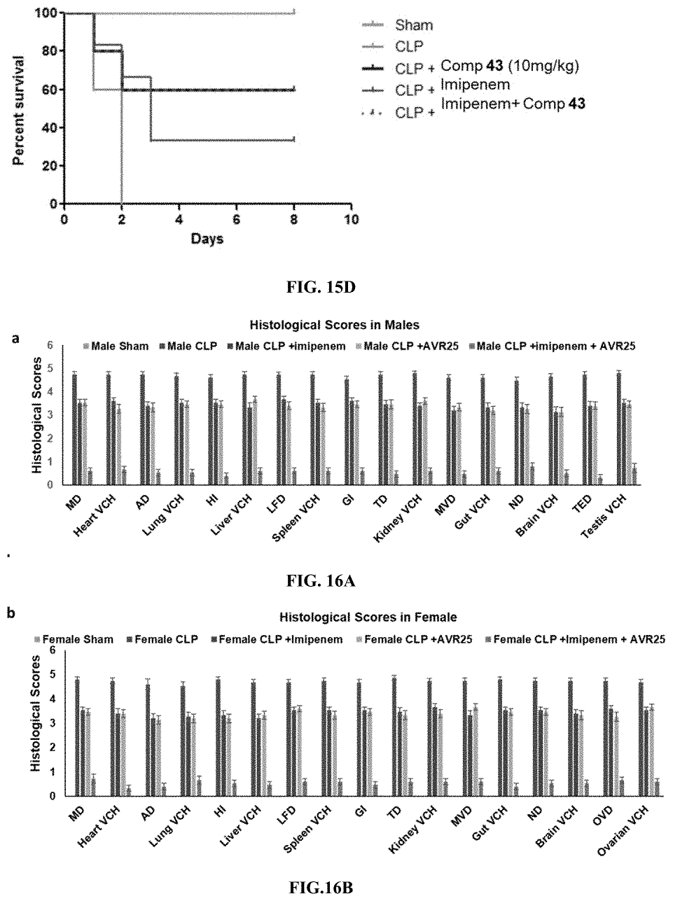

Compounds of the present invention have shown unexpectedly superior activity in inhibiting LPS induced inflammation biomarkers (TNF-α, IL-1β and IL-6) in human peripheral blood mononuclear cells. As illustrated in FIGS. 15A-D, Compounds 34 demonstrated high efficacy in protecting organ dysfunction and death of mice in a cecal ligation and puncture (CLP) model of sepsis at 10 mg/kg on intravenous (IV) dosing and downregulated inflammatory cytokines such as TNF-α, IL-1β and IL-6 in a statistically significant manner. Compound 34 also reduced both acute and chronic lung injuries in mouse models via IP injection.

Compounds of the present invention (34) can be synthesized using reported procedure as describe in Mohamed R. E et al, Carbohydrate research, 2001, 331, 129-142.

Accordingly, the compounds find use as anti-inflammatory compounds. Compounds of the present invention (35-38) can be synthesized using the synthesis schemes illustrated in FIG. 31 in conjunction with knowledge available in the art.

Additional compounds of the present disclosure include the following structures shown below where:

n=0-7, X=O, Y=O, R=H, R₂=H, C(O)CH₃, R₅=Aryl, and substituted aryls as shown here

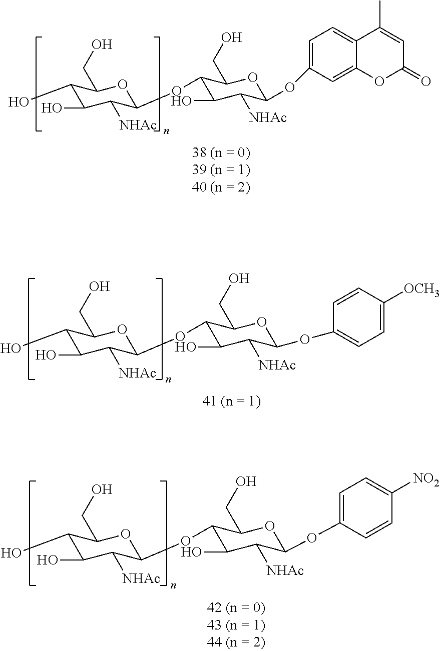

Compounds of the present invention (38-44) have shown to inhibit LPS induced inflammation biomarkers (TNF-α, IL-1β and IL-6) in human monocytes and upregulated anti-inflammatory cytokine, M2 biomarker IL-10. Compound 39, 42 and 43 also showed broad spectrum antimicrobial activity against gram-negative, gram-positive bacteria as well fungus. Compound 39 and 43 unexpectedly inhibited biofilm formation by MSSA and MRSA. Compounds 39, 42 and 43 demonstrated high survival, organ protection in CLP mice model of sepsis when administered intravenously (10 mg/kg dose). Accordingly, compounds as described herein find use as anti-inflammatory molecules in some embodiments. In some embodiments the compounds are anti-infective or antimicrobial.

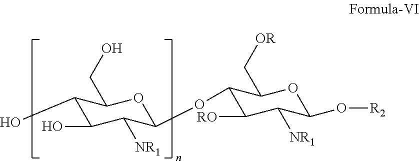

where:
n=0-1
R=benzyl, substituted benzyl
R₁=COCH₃, N-dimethylmaleimide
R₂=cyclohexyl, p-nitro phenyl, piperidine nitroxy, piperidine-N-hydroxyl, p-methoxy phenyl.

Additional compounds of the present invention include the following structures shown below:

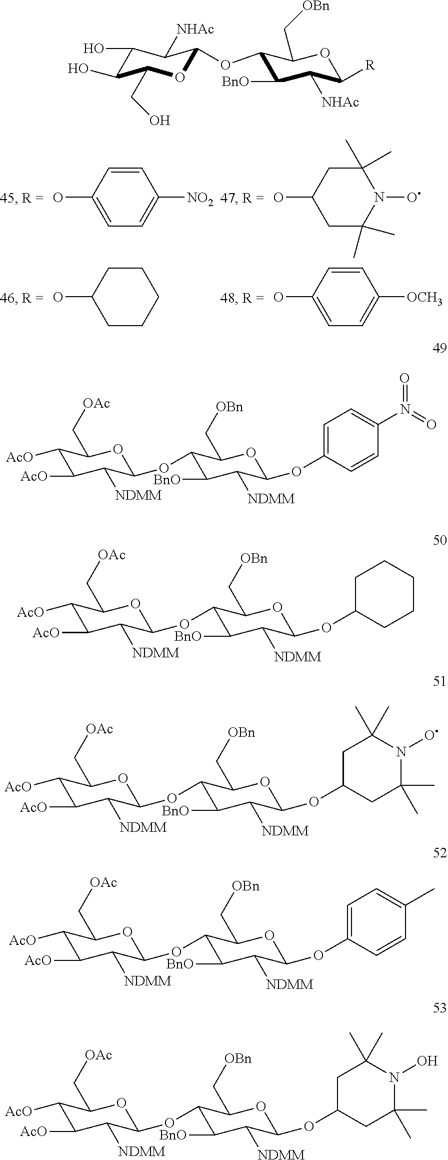

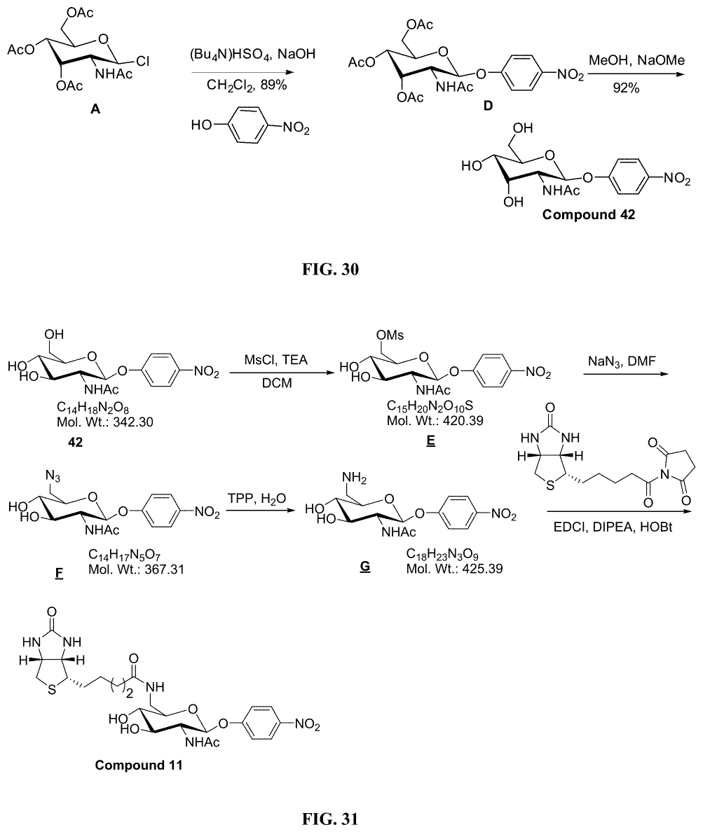

Compounds of the present invention have shown to inhibit LPS induced inflammation biomarkers (TNF-α, IL-1β and IL-6) in human monocytes and upregulated IL-10. Compounds 49 also showed broad spectrum antimicrobial activity against gram-negative, gram-positive bacteria as well fungus. Compounds 49 unexpectedly inhibited biofilm inhibition by MSSA and MRSA. Compound 49 demonstrated high survival, organ protection in CLP mice model of sepsis when administered intravenously (5.0 mg/kg dose). Compounds of the present invention can be synthesized using the synthesis schemes illustrated in FIG. 30-31 developed by us in conjunction with knowledge available in the art.

Furthermore, certain embodiments comprise pharmaceutically acceptable salts of compounds according to the present invention. Pharmaceutically acceptable salts comprise, but are not limited to, soluble or dispersible forms of compounds according to the present invention that are suitable for treatment of disease without undue undesirable effects such as allergic reactions or toxicity. Representative pharmaceutically acceptable salts include, but are not limited to, acid addition salts such as acetate, citrate, benzoate, lactate, or phosphate and basic addition salts such as lithium, sodium, potassium, or aluminum.

Formulations

In some embodiments, the compounds of the present disclosure are incorporated into parenteral formulations. The term parenteral as used herein includes subcutaneous, intravenous, intramuscular, and intra-arterial injections with a variety of infusion techniques. Intra-arterial and intravenous injection as used herein includes administration through catheters. Preferred for certain indications are methods of administration that allow rapid access to the tissue or organ being treated, such as intravenous injections for the treatment of endotoxemia or sepsis.

The compounds of the present disclosure will be administered in dosages which will provide suitable inhibition of LPS activation of target cells; generally, these dosages are, preferably between 50-3000 mg/patient, or from 100-2500 mg/patient or from 200-2000 mg/patient or from 500-1000 mg/patient or from 750-1000 mg/patient, more preferably, between 500-750 mg/patient and most preferably, between 250-500 mg/patient. The dosages are preferably once a day for 28 days, more preferably twice a day for 14 days or most preferably 3 times a day for 7 days.

Pharmaceutical compositions containing the active ingredient may be in any form suitable for the intended method of administration.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropylmethyl cellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadeaethyleneoxycetanol), a condensation product of ethylene oxide with a partial ester derived from a fatty acid and a hexitol anhydride (e.g., polyoxyethylene sorbitan mono-oleate). The aqueous suspension may also contain one or more preservative such as ethyl of n-propyl p-hydroxybenzoate.

The pharmaceutical compositions of the invention are preferably in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenteral-acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

In some embodiments the formulation comprises PLA or PLGA microparticles and may be further mixed with Na₂HPO₄, hydroxypropyl methylcellulose, polysorbate 80, sodium chloride, and/or edentate disodium.

Formulations suitable for parenteral administration include aqueous and non-aqueous isotonic sterile injection solutions which may contain anti-oxidants, buffers, bacteriostats and solutes which render the formation isotonic with the blood of the intended recipient; and aqueous and non-aqueous sterile suspensions which may include suspending agents and thickening agents. The formulations may be presented in unit-dose or multi-dose sealed containers, for example, ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders of the kind previously described.

It will be understood, however, that the specific dose level for any particular patient will depend on a variety of factors including the activity of the specific compound employed; the age, body weight, general health, and sex of the individual being treated; the time and route of administration; the rate of excretion; other drugs which have previously been administered; and the severity of the particular disease undergoing therapy.

In some embodiments the compositions of the present disclosure also contain from about 80% to about 99.5%, preferably from about 90 or 95% to about 98.5% of a compatible non-aqueous pharmaceutically acceptable topical vehicle. Some vehicles are described in U.S. Pat. No. 4,621,075, which is incorporated herein for this disclosure. Although it is preferred that these vehicles be free of water, the compositions of the present invention may contain up to about 5% water without significant adverse effects on the formation of the desired gels. These non-aqueous vehicle components are also well-known in the pharmaceutical arts, and they include (but are not limited to) short chain alcohols and ketones and emollients, such as hydrocarbon oils and waxes, lanolin and lanolin derivatives, silicone oils, monoglyceride, diglyceride, and triglyceride esters, fatty alcohols, alkyl and alkenyl esters of fatty acids, alkyl and alkenyl diesters of dicarboxylic acids, polyhydric alcohols and their ether and ester derivatives; wax esters and beeswax derivatives. Preferred vehicles incorporate methanol, ethanol, n-propanol, isopropanol, butanol, polypropylene glycol, polyethylene glycol and mixtures of these components. Particularly preferred vehicles include ethanol, n-propanol and butanol, especially ethanol. These preferred solvents may also be combined with other components, such as diisopropyl sebacate, isopropyl myristate, methyl laurate, silicone, glycerine and mixtures of these components, to provide non-aqueous vehicles which are also useful in the present invention. Of these additional components, diisopropyl sebacate is especially useful. In fact, preferred vehicles include mixtures of ethanol and diisopropyl sebacate in ratios, by weight, of from about 4:1 to about 1:4. Preferred vehicles contain from about 15% to about 35% diisopropyl sebacate and from about 65% to about 85% ethanol.

Compositions of the present invention may additionally contain, at their art-established usage levels, compatible adjunct components conventionally used in the formulation of topical pharmaceutical compositions. These adjunct components may include, but are not limited to, pharmaceutically-active materials (such as supplementary antimicrobial or anti-inflammatory ingredients, e.g., steroids) or ingredients used to enhance the formulation itself (such as excipients, dyes, perfumes, skin penetration enhancers, stabilizers, preservatives, and antioxidants). Since the compositions of the present invention permit the formation of gels without requiring the presence of conventional gelling agents, such agents are preferably not included. Examples of such agents include the pharmaceutically-acceptable acidic carboxy polymers, such as the Carbopol compounds commercially available from B. F. Goodrich Chemicals, Cleveland, Ohio.

The gel-form compositions of the present invention may be formulated by the conventional mixing of the components described above. Gel formation takes place within from about 2 minutes to about 16 hours after mixing, depending upon the components utilized.

In one embodiment the cream, lotion or gel packaged in a common trigger spray container will be firmly adhered to the area of interest as a regular cream does after it is sprayed out from the container. This is described in WO 98/51273, which is incorporated herein by reference. Accordingly, in one aspect, the present disclosure provides a pharmaceutical non-aerosol spray composition for topical application, which comprises the compounds as described herein alone or in combination. The compounds are present in an amount in the range of 0.1% to 20% or in some embodiments from 1 to 15% by weight, or in some embodiments from 2 to 10% by weight of cream, lotion or gel. The compounds used in the present invention can be incorporated into a neutral hydrophilic matrix cream, lotion or gel. In a first preferred embodiment, the cream or lotion matrix for topical application is characterized by polyoxyethylene alkyl ethers. In a second preferred embodiment, the gel is characterized by high molecular weight polymer of cross-linked acrylic acid. Polyoxyethylene alkyl ethers are non-ionic surfactants widely used in pharmaceutical topical formulations and cosmetics primarily as emulsifying agents for water-in-oil and oil-in-water emulsions. It is characterized in this invention as a base for non-aerosol trigger sprayable cream or lotion. Cross-linked acrylic acid polymer (Carbomer) employed to form the gel is another object of this invention.

A particularly suitable base for non-aerosol spray is therefore a cream or lotion containing from 1 to 25% of polyoxyethylene alkyl ethers, 3 to 40% of humectant and 0.1 to 1% of preservative or preservatives and the balance to 100% being purified water. Aptly the polyoxyethylene alkyl ether can be one or any combination selected from the group consisting of polyoxyl 20 cetostearyl ether (Atlas G-3713), poloxyl 2 cetyl ether (ceteth-2), poloxyl 10 cetyl ether (ceteth-10), poloxyl 20 cetyl ether (ceteth-20), poloxyl 4 lauryl cetyl ether (laureth-4), poloxyl 23 lauryl cetyl ether (laureth-23), poloxyl 2 oleyl ether (oleth-2) poloxyl 10 oleyl ether (oleth-10), poloxyl 20 oleyl ether (oleth-20), poloxyl 2 stearyl ether (steareth-2), poloxyl 10 stearyl ether (steareth-10), poloxyl 20 stearyl ether (steareth-20) and poloxyl 100 stearyl ether (steareth-100). Suitable humectant can be one or any combination selected from the group consisting of propylene glycol, polyethylene glycol, sorbitol or glycerine. Suitable preservative is one or any combination selected from the group consisting of methylparaben, propylparaben, benzyl alcohol, benzoic acid, sodium benzoate, sorbic acid and its salt or phenylethyl alcohol.

Another suitable base for non-aerosol spray is a gel containing from 0.1 to 2.0% of Carbomer, 0.1 to 1% of alkaline solution, 3 to 40% of humectant and 0.1 to 1% of preservative or preservative as and the balance to 100% being purified water. Aptly the Carbomer can be one or any combination selected from the group consisting of Carbomer 934, Carbomer 940 or Carbomer 941. The suitable humectant, preservative and purified water for the gel are same as that in the case or cream or lotion. Other sprayable formulations are described in US Pre-Grant Publication US2005/00255048, which is expressly incorporated herein by reference.

Ophthalmic Formulation (Topical and Intravitreal Dosing):

The compound of the invention will typically be a small percentage of the total ophthalmic composition. The compound of the invention will typically be at least 0.01 w/v %, more typically at least 0.1 w/v % and even more typically at least 0.5 w/v 3% of the ophthalmic composition. The compound of the invention will also typically be no greater than 5.0 w/v %, even more typically no greater that 3.0 w/v % and even more typically no greater than 1.5 w/v % of the ophthalmic composition.

The ophthalmic composition will also typically include a suitable ophthalmic vehicle for delivery of the compound to the eye. It is contemplated that the ophthalmic composition may be configured for topical or intravitreal application to the eye and the ophthalmic vehicle will likely be different depending upon the manner of application. Generally, for either topical or intravitreal applications, it is preferable that the ophthalmic composition be aqueous and include a substantial amount of water. Typically, the composition will include at least 30 w/v %, more typically at least 80 w/v 3% b and even more typically at least 90 w/v % water (e.g., purified water).

For intravitreal applications, particularly when the ophthalmic composition is applied to the eye with a syringe, the ophthalmic compositions may include only or consist essentially of water and compound of the invention. For sustained drug release, PLGA or PLA macroparticle formulation of the compound of invention will be used as described by Shelke et al [Drug Deliv Transi Res. 2011, (1): 76-90]. Of course, the ophthalmic composition could include other ingredients as well such as Na₂HPO₄, hydroxypropyl methylcellulose, polysorbate 80, sodium chloride, and edentate disodium.

it could also be the case that the vehicle be only or consist essentially of water for a topical application, particularly if that topical application is performed shortly after water is combined with the test compound or the composition is packaged in a manner to prevent contamination. However, if the ophthalmic composition is to be applied as a multi-dose ophthalmic composition over an extended period of time (e.g., as drops from an eye-dropper once, twice, thrice or more per day for multiple days), the ophthalmic composition will likely include additional ingredients such as antimicrobial or preservative agents or systems, surfactants, buffering agents, tonicity agents, anti-oxidants, viscosity-modifying agents any combinations thereof or the like.

For topical application, the compositions of the present invention typically include antimicrobial agent. Potential antimicrobial agents include, without limitation, hydrogen peroxide, chlorine containing preservatives such as benzalkonium chloride or others. According to a preferred aspect, however, the composition of the present invention is entirely or substantially free of any non-polymeric quaternary anti-microbial agents such as benzalkonium chloride (BAK). Most preferred antimicrobial agent in the pharmaceutical composition includes polymeric quaternary ammonium compound.

As used herein, the phrase “substantially free of” as it refers to an ingredient of the ophthalmic composition means that it is contemplated that the ophthalmic composition can be either entirely devoid of that particular ingredient or includes only a nominal amount of that particular ingredient.

The polymeric quaternary ammonium compounds useful in the compositions of the present invention are those which have an antimicrobial effect and which are ophthalmically acceptable. Preferred compounds of this type are described in U.S. Pat. Nos. 3,931,319; 4,027,020; 4,407,791; 4,525,346; 4,836,986; 5,037,647 and 5,300,287; and PCT application WO 91/09523 (Dziabo et al.), which are expressly incorporated herein by reference. The most preferred polymeric ammonium compound is polyquaternium 1, otherwise known as POLYQUAD™ or ONAMERM™ with a number average molecular weight between 2,000 to 30,000. Preferably, the number average molecular weight is between 3,000 to 14,000.

The polymeric quaternary ammonium compounds are generally used in the suspensions of the present invention in an amount that is greater than about 0.00001 w/v %, more typically greater than about 0.0003 w/v % and even more typically greater than about 0.0007 w/v % of the suspension. Moreover, the polymeric quaternary ammonium compounds are generally used in the compositions of the present invention in an amount that is less than about 3 w/v %, more typically less than about 0.003 w/v % and even more typically less than about 0.0015 w/v % of the composition.

The antimicrobial agent of the composition of the present invention can additionally or alternatively include an antimicrobial system such as a borate/polyol complex system. As used herein, the term “borate” shall refer to boric acid, salts of boric acid, borate derivatives and other pharmaceutically acceptable borates, or combinations thereof. Most suitable are: boric acid, sodium borate, potassium borate, calcium borate, magnesium borate, manganese borate, and other such borate salts. Borate interacts with polyols, such as glycerol, propylene glycol, sorbitol and mannitol, to form borate polyol complexes. The type and ratio of such complexes depends on the number of OH groups of a polyol on adjacent carbon atoms that are not in trans configuration relative to each other. It shall be understood that weight/volume percentages of the ingredients polyol and borate include those amounts whether as part of a complex or not.

As used herein, the term “polyol” includes any compound having at least one hydroxyl group on each of two adjacent carbon atoms that are not in trans configuration relative to each other. The polyols can be linear or cyclic, substituted or unsubstituted, or mixtures thereof, so long as the resultant complex is water soluble and pharmaceutically acceptable. Examples of such compounds include: sugars, sugar alcohols, sugar acids and uronic acids. Preferred polyols are sugars, sugar alcohols and sugar acids, including, but not limited to: mannitol, glycerin, xylitol, sorbitol and propylene glycol.

When used, the borate/polyol complex antimicrobial system (i.e., the borate and polyol together) typically comprise at least 0.05 w/v %, more typically at least 0.5 w/v % and even possibly at least 1 or even at least 1.2 w/v % of the composition and also typically comprise less than 5 w/v %, more typically less than 2.2 w/v % and even possibly less than 1.6 w/v % of the composition. The borate to polyol ratio (weight to weight ratio) in the composition is typically between 1 to 1 and 1 to 10 and more typically is between 1 to 2 and 1 to 4 (e.g., about 1 to 3).

Tyloxapol, polysorbate-80 and polyoxyl hydrogenated castor oil are preferred surfactants. Tyloxapol is a highly preferred surfactant. When used, the surfactant is typically present in a concentration that is at least 0.01 w/v %, more typically at least 0.025 w/v % and even possibly at least 0.1 w/v % of the composition and also typically is less than 5 w/v %, more typically less than 2.0 w/v % and even possibly less than 1.0 w/v % of the composition.

The compositions of the present invention that are to be used for topical applications are typically formulated so as to be compatible with the eye. The ophthalmic compositions intended for direct application to the eye will be formulated so as to have a pH and tonicity that are compatible with the eye. The compositions will typically have a pH in the range of 4 to 9, preferably 5.5 to 8.5, and most preferably 5.5 to 8.0. Particularly desired pH ranges are 6.0 to 7.8 and more specifically 6.4 to 7.6. The compositions will have an osmolality of 200 to 100 or 450 milliosmoles per kilogram (mOsm/kg), more preferably 240 to 360 mOsm/kg.

Preferred compositions of the present invention are multi-dose ophthalmic compositions, for example, where the composition is in an eye dropper and can be administered as one or more drops once, twice, thrice or more times per day, topically to the eye. In that case, the compositions preferably have sufficient antimicrobial activity to allow the compositions to satisfy the USP preservative efficacy requirements, as well as other preservative efficacy standards for aqueous pharmaceutical compositions.

The preservative efficacy standards for multi-dose ophthalmic solutions in the U.S., and other countries/regions are set forth in the following table:

Preservative Efficacy Test (“PET”) Criteria
(Log Order Reduction of Microbial Inoculum Over Time)

| | Bacteria | Fungi |
|---|---|---|
| USP 27 | A reduction of 1 log | The compositions must |
| | (90%), by day 7; | demonstrate over the entire test |
| | 3 logs (99.9%) by | period, which means no increases |
| | day 14, and no increase | of 0.5 logs or greater, relative to |
| | after day 14 | the initial inoculum. |
| Japan | 3 logs by 14 days; and | No increase from initial count at |
| | no increase from day 14 | 14 and 28 days |
| | through day 28. | |
| Ph. Eur. A¹ | A reduction of 2 logs | A reduction of 2 logs (99%) by 7 |
| | (99%) by 6 hours; 3 logs | days, and no increase thereafter |
| | by 24 hours; and no | |
| | recovery after 28 days | |
| Ph. Eur. B | A reduction of 1 log at | A reduction of 1 log (90%) by |
| | 24 hours; 3 logs by | day 14, and no increase thereafter |
| | day 7; and no increase | |
| | thereafter | |
| FDA/ISO | A reduction of 3 logs | No increase higher than the initial |
| 14730 | from initial challenge at | value at day 14, and no increase |
| | day 14; and a reduction | higher than the day 14 |
| | of 3 logs from | rechallenge count through |
| | rechallenge | day 28. |

There are two preservative efficacy standards in the European Pharmacopoeia “A” and “B”.

The standards identified above for the ISP 27 are substantially identical to the requirements set forth in prior editions of the USP, particularly USP 24, USP 25 and USP 26.

As an added advantage, these ophthalmic compositions containing TLR4 antagonist compounds of the present invention are suitable for topical applications to the eye.

The formulations described herein may also contain additional active ingredients, such as but not limited to anti-microbial agents as described above, pain reducing agents and the like.

As such, once made, the compounds and formulations described herein find use in the treatment of a variety of ocular inflammatory disorders including, but not limited to, AMD, sepsis and severe sepsis, SIRS and septic shock and the like. The methods comprise administering to a patient in need thereof an effective amount of the antimicrobial and anti-inflammatory compositions described herein such that the disease or disorder is treated. The medical use of such compounds will be for the treatment and/or management of sepsis, neonatal sepsis, septicemia, septic shock, burn and wounds, infective endocarditis, biofilm inhibition, ocular infection, ocular inflammation, ocular angiogenesis, diabetic retinopathy, retinopathy of prematurity, uveitis, rheumatoid arthritis (RA), atherosclerosis, inflammatory bowel diseases (IBD), asthma, acute respiratory distress syndrome, chronic obstructive pulmonary disease, bronchopulmonary dysplasia, fever syndromes, cachexia, psoriasis, autoimmune diseases, cardiac diseases, retinoblastoma, cancer, acute and chronic kidney injury and/or any disorder associated with inflammation, immunomodulation and microbial infection.

### Examples

The following examples, including the experiments conducted and results achieved are provided for illustrative purposes only and are not to be construed as limiting the invention.

EXAMPLE 1. In vitro and in vivo efficacy: The compounds of invention reduced CFUs of Pseudomonas aeruginosa bacteria in mouse lungs, increased phagocytotic activity in human macrophages and demonstrated synergistic activities with standard of care antibiotics.

FIG. 1. Female CD-1 mice (16 weeks of age) were housed in microisolator cages (n=4) for two weeks prior to infection. Anesthetized mice were infected with 10⁵ CFUs of P. aeruginosa in 40 μl by intranasal inoculation. Eight hours after infection, selected groups of mice (n=4) were dosed with either meropenem (1.5 mg/kg) via intraperitoneal injection, Compound 1 or 4 (10 mg/kg), or meropenem+compound 1 or 4 in 200 μl saline IV. Data represent the mean CFUs in lung at 72 h between treatment groups. Individual observations represent the number of mice remaining at time of sacrifice; n=5 for all groups. The experiment was repeated two times.

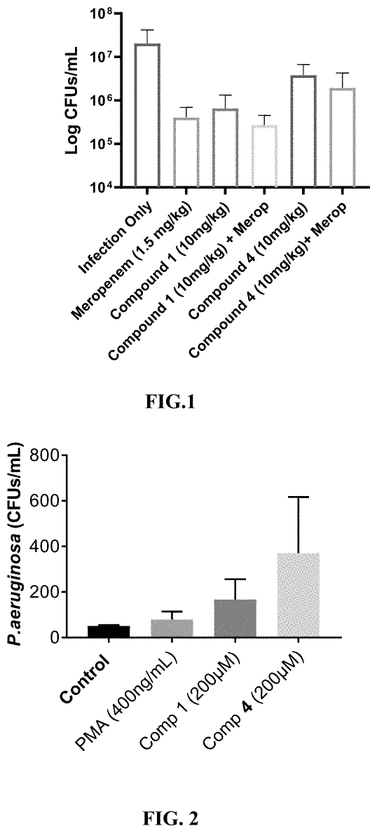

FIG. 2. Human leukemic THP-1 monocytic cells (10⁵) treated with phorbol myristyl acetate PMA (200 ng/ml) or 200 μM of Compounds 1 or 4 for 72 h to produce macrophages. Increase in phagocytosis of GFP tagged P. aeruginosa (treated 1:20 ratio of cells:bacteria) was observed after 0.5 h of incubation and treatment with gentamycin (100 μg/ml) to remove extracellular bacteria. The cell lysate was plated in agar and CFUs were calculated.

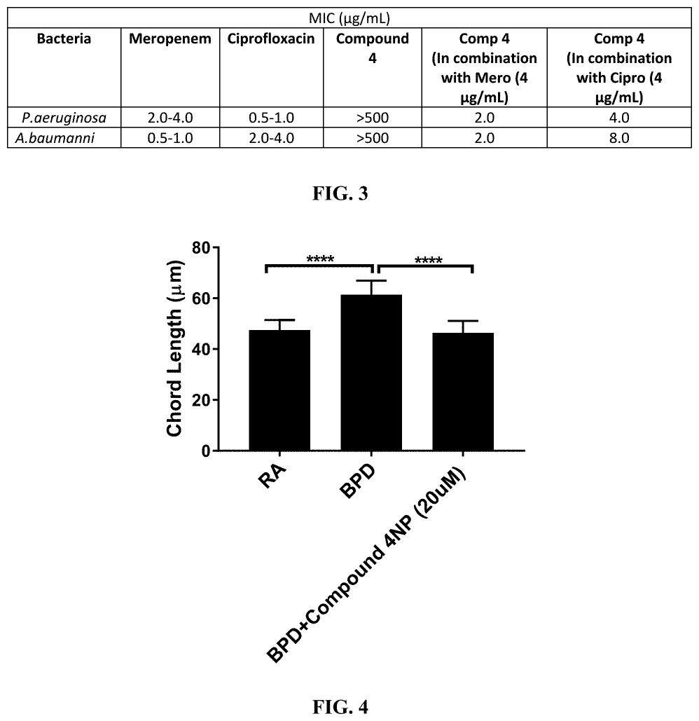

FIG. 3: Checkerboard MIC assay of Compound 4+different SoC antibiotic combinations using gram-negative (P. aeruginosa, A. baumanni) bacteria. To determine the synergy, Compound 4 was added horizontally diluted two-fold from left to right where antibiotics were added from top to bottom at their MIC, ½ MIC and at ¼ MIC concentration in a 96 well plate. 5 μl of bacteria was added to each well and incubated for 6 h and the plate was read at 600 nm. The MIC₉₀ was calculated. N=4. The last two columns show the combination MIC of compound 4 illustrating the synergy observed. Fractional inhibitory concentration was found to be <0.5 for the combinations.

EXAMPLE 2. The compounds of present invention prevent lung injury in a mouse model. FIG. 4 is a graph that shows the effect of intranasal PLGA nanosuspension of compound 4 (20 μM, at day P2 and P4) on normoxia (21% O₂, room air, RA, P0-P14), and BPD mouse pups (100% O₂, P0-P4, then P4-P14 at 21% O₂). In RA pups, all the compounds were safe to use with no significant change in chord lengths (CL), an indicator of lung injury. In BPD pups, the CL significantly increased indicating abnormal lung development in BPD groups. After treatment with compound 4 the CL of the pups significantly reduced as compared to the BPD groups. N=4-5. ****p<0.0001. One-way ANOVA, Dunnett's multiple comparisons test.

EXAMPLE 3. In vitro genotoxicity, hERG channel and off target binding effect of the compounds of present inventions

FIG. 5: Ames bacterial reverse mutation test was conducted at Covance Inc, Harrogate, UK according to their screening protocol in the absence and presence of S-9 (Jemnitz, Veres et al. 2004). Treatments of compounds 1, 4, 34 and 42 (3.2, 10, 32, 100, 320, and 1000 μg/well) were conducted in strains TA 98 and TA100 (Salmonella typhimurium) in the absence and presence of metabolic activation system S-9 (Molecular Toxicology Incorporated, USA) from male Sprague-Dawley rats induced with Aroclor 1254 added as a 5% mix to the test system. The cultures were incubated at 37±1° C. to provide a working culture of approximately 108 to 109 cells/mL, which was confirmed by either viability plating or optical density (OD) assessment at 650 nm. Revertant counts per well and the mean number of revertants (per well) were calculated for each treatment and strain. Sodium azide (NaN₃, 0.3 μg/well) was used as a positive control for the S-9 experiment and 2-Aminoanthracene (AAN, 0.4 μg/well) was used for the +S-9 experiment to activate mutagenesis. There was no evidence of cytotoxicity of compounds 1, 4, 34 and 42 for any strain and no increase in the number of revertant colonies with or without metabolic activation indicating that the compounds do not increase the mutation rate.

FIG. 5 also shows an alternative to measuring structural aberrations in mitotic cells is to measure micronuclei in human peripheral blood lymphocytes. These are produced from acentric fragments or whole chromosomes that are unable to attach to the spindle at mitosis and appear during the next interphase as small bodies adjacent to the main daughter nucleus. Cytochalasin B (Cyto-B), if added to the cultures, inhibits cytokinesis (cell division) but not karyokinesis (nuclear division) resulting in the formation of binucleate cells (Fenech and Morley 1985). If micronuclei are counted in binucleate cells, then a measurement of micronucleus induction resulting from cell division can be obtained. In the absence of S-9 activation, no statistically significant increases in MNBN cells were observed for any concentration of compounds 1, 4, 34 and 42 were analyzed. The MNBN cell frequency of all test article treated cultures fell within the normal range. Likewise, in the presence of S-9 activation, no statistically significant increases in MNBN cells were observed for any concentration of compounds 1, 4, 34 and 42 analyzed. The MNBN cell frequency of all test article treated cultures fell within the normal range. Thus, all the tested compounds do not induce any micronucleus formation at doses of 343-500 μg/mL.

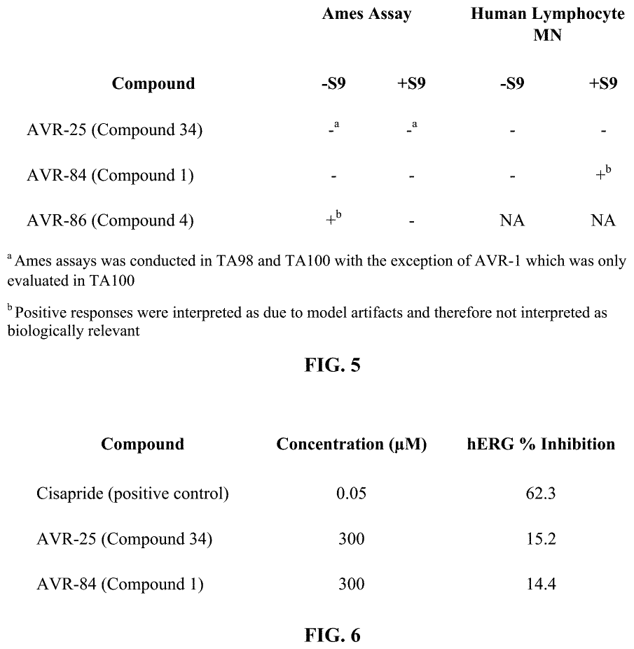

FIG. 6: Briefly, in vitro effects of compounds 1, 34 and 42 on the hERG (human ether-à-go-go-related gene) potassium channel current (a surrogate for IKr, the rapidly activating, delayed rectifier cardiac potassium current) expressed in HEK293 cells was evaluated at room temperature using the QPatch HT® (Sophion Bioscience A/S, Denmark), an automatic parallel patch clamp system. The test articles were evaluated at 0.3, 3, 30, and 300 μM, with each concentration tested in three sets of cells (N=3). The duration of exposure to each compound concentration was approximately 5 minutes. Cisapride (0.05 μM) was used as a positive control to confirm the sensitivity of the test system to hERG inhibition. Compounds AVR-25 (compound 34) compound X), AVR-48 (compound 42) and AVR-84 (compound 1) demonstrated a negligible to minimal effect on hERG activity at a concentration of 300 μM with 1-20% inhibition of the hERG channel function as shown in FIG. 6 where the positive control cispride at 0.05% inhibited 62% of hERG channel activity.

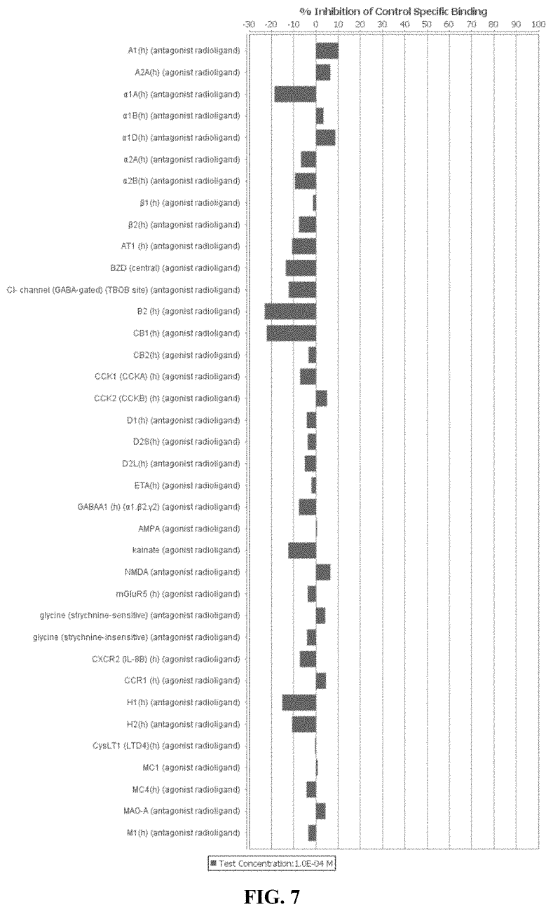

FIGS. 7 and 8: This study was conducted at Eurofins to establish the potential compounds 4 and 42 to interact with 87 different off-target receptors, ion channels, transporters, and enzymes. Compound 4 tested at 100 μM was analyzed in enzyme or radioligand binding assays according to standard protocols adapted from the scientific literature to maximize reliability and reproducibility. Reference standards were run as an integral part of each assay to ensure the validity of the results obtained. All results from the assay were expressed in terms of that assay's quantitation method and a level of ≥50% inhibition or stimulation for biochemical assays were considered significant responses. No significant results were noted. The results of this study suggest that compounds 4 and 42 has no or low propensity for off-target interactions up to the high 100 μM concentration tested with <20% binding affinity.

EXAMPLE 4. Pharmacokinetic study of the compounds of present inventions in rats.

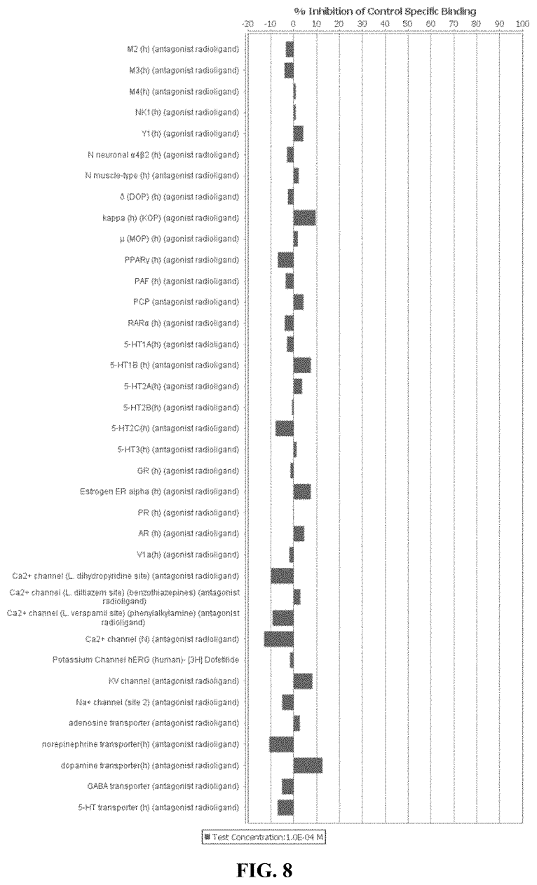

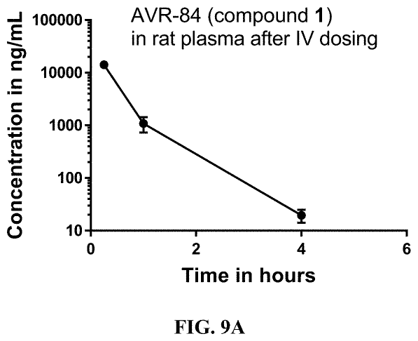

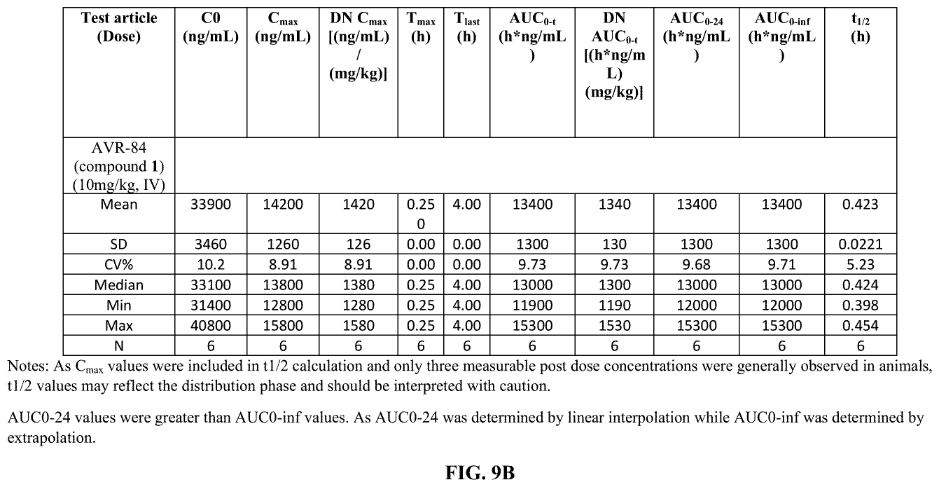

FIGS. 9A and 9B: This study was designed to provide a pharmacokinetic profile of tested compounds in both male and female Sprague Dawley rat plasma over 24 hours (0.25, 1, 4, 8, and 24 hours), individually delivered via IV route at a dose of 10 mg/kg, as determined by LC-MS/MS. The study was conducted in Covance laboratories, Harrogate, UK following their approve animal protocol. Six compounds were tested at 10 mg/kg IV injection for this study. After a single intravenous dose of 10 mg/kg of AVR-84 (compound 1), concentrations rapidly declined, with mean t_{1/2} values of 0.423 hour and mean concentrations measurable through 4.00, 8.00, and 4.00 hours, respectively. The exposure to AVR-84, as assessed by mean Cmax and AUC0-t values following IV injection of 10 mg/kg. The mean AUC₀₋ₜ value AVR-84 was found to be 13400 ng h/mL. Sex differences in AVR-84 mean Cₘₐₓ and AUC₀₋ₜ values were less than 2-fold. Other 5 compounds showed similar PK profile.

EXAMPLE 5. A series of compounds of present invention binds to both TLR4 and CD163 receptor in macrophages.

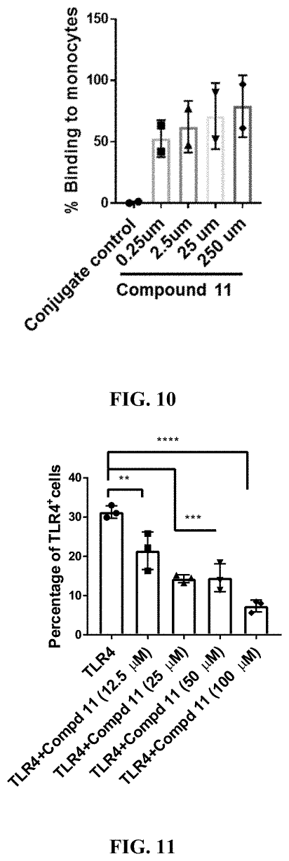

FIG. 10: Binding of biotinylated compound 11 to splenic monocytes/macrophages derived from primary splenocytes of C57BL/6J mice (n=3-5). Briefly, cells were incubated at 4° C. for 1 hr followed by incubation with biotinylated compound 11 (0.25 μM, 2.5 μM, 25 μM, and 250 μM) along with monocyte (Ly6C) markers. Then the cells were probed with appropriate fluorescence coupled streptavidin and analyzed by FACS. Dead cells were excluded during analysis. N=3

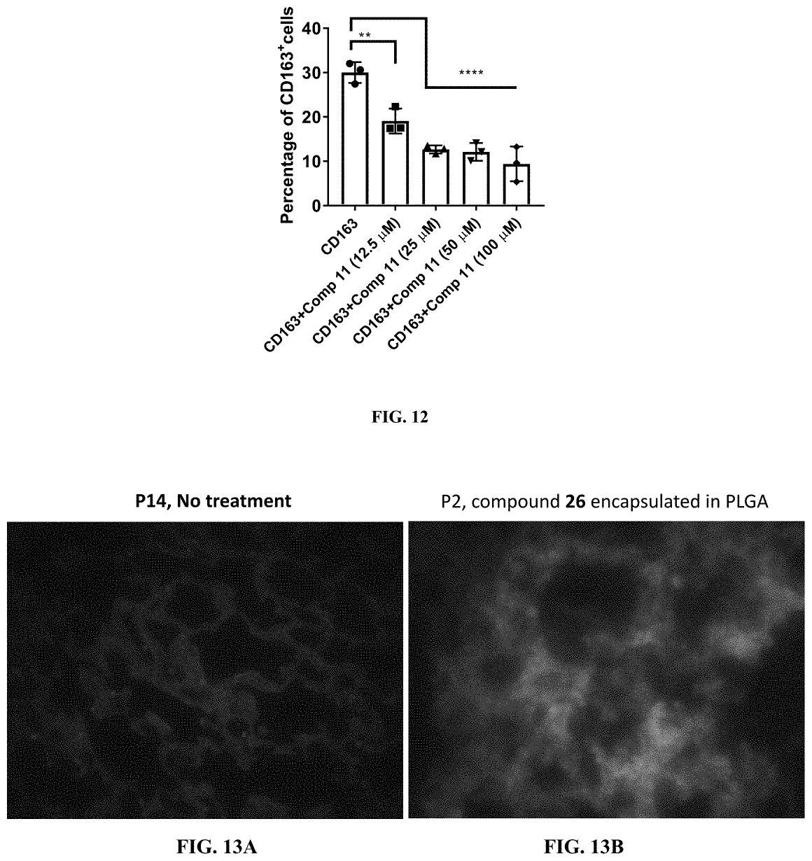

FIGS. 11 and 12: Cells were incubated at 4° C. for 1 hr followed by incubation with biotinylated compound 11 along with monocyte markers. Then the cells were probed with appropriate fluorescence coupled streptavidin (for anti-human TLR4 and CD163 antibodies) and analyzed by FACS. N=5

EXAMPLE 6. The compound of present invention reaches the lungs when delivered via intranasal (IN) drop and decreased lung injury in BPD mouse model.

FIGS. 13A and 13B: Compound 26 with FITC group as fluorescence marker was encapsulated in PLGA and delivered as IN (20 μM) suspension to mouse pups. Cryosections of the control mouse lungs show the absence of the drug (no fluorescence) and presence of compound 26 with fluorescence. RA: room air; FITC: fluorescein isothiocyanate; PLGA: poly D, L-lactic-co-glycolic acid; Scale bar 100 μm, N=2.

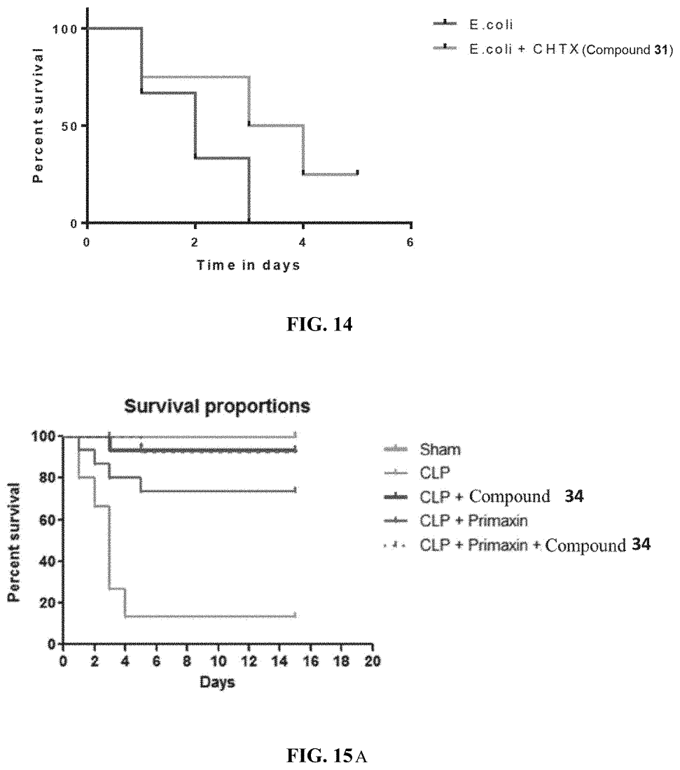

FIG. 14 is a graph showing the results of an evaluation of the TLR4 modulator compound 31 of the present invention in a mouse endotoxemia sepsis model and illustrates that compound 31 protected mice from lethal gram-negative sepsis against E. coli at a dose of 10 mg/kg.

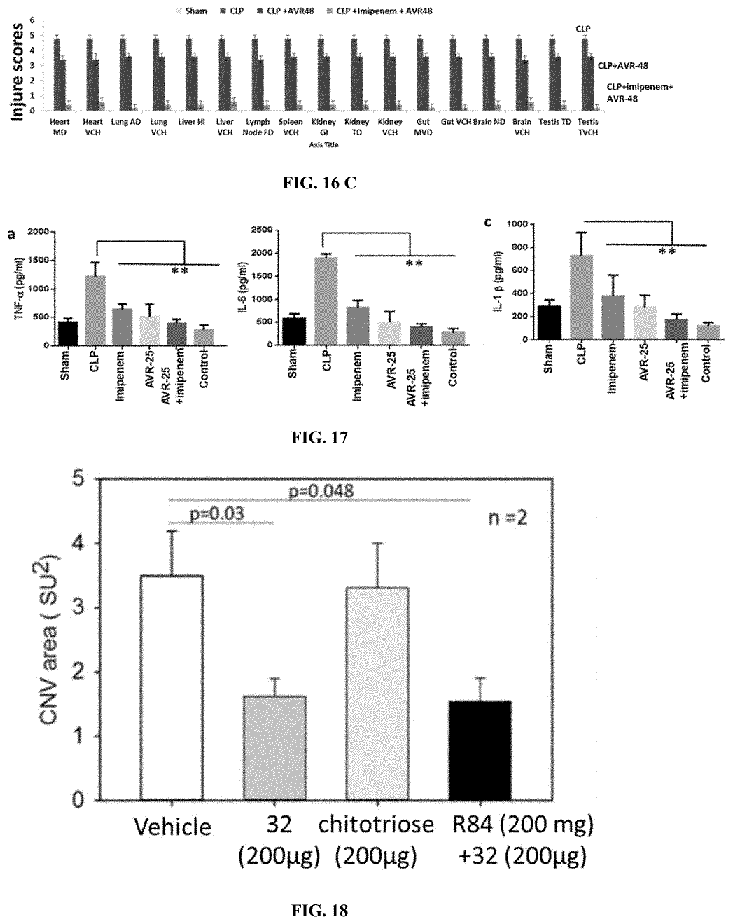

FIGS. 16A to 16C are the histopathology score of major organs which demonstrates that, on treatment of compounds 34 and 44, the compounds of present invention reversed the major pathological changes and tissues resembled to sham group.

EXAMPLE 7. Compounds were tested for inhibiting the production of inflammatory mediators in human peripheral blood mononuclear cells.

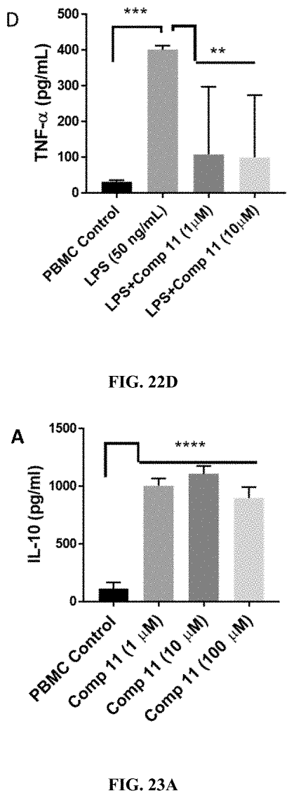

FIGS. 22A to 22D: To understand the structural requirement and limitations to probe the TLR4 binding pocket for optimal potency and efficacy, we have studied the ability of chitooligomers to inhibit LPS induced inflammation in human peripheral blood mononuclear cells. Compounds 31-34 inhibited LPS induced cytokines TNF-α, IL-1β and IL-6) in a statistically significant manner at 10 μM concentration (FIGS. 22A-22C). Compound 32 and 34 (10 μM) were found to be more potent than chitohexaose, (Compound 31) (10 μM) in terms of percentage of inhibition of LPS mediated induction of inflammatory cytokines (LPS vs Chtx p<0.001 whereas LPS vs compound 32 or 34 p<0.0001). The protocol was followed as described [Panda et al, PLoS Pathog 2012, 8, e1002717]. Human mononuclear cells were stimulated with LPS along with the series of compounds (10 μM) for 48 h. TNF-α, IL-1β, Il-6 in culture supernatants were quantified according to the manufacturer's instruction. Similarly, other analog 11 was assessed for their ability to inhibit LPS induced TNF-α production in PBMC after 24 h of treatment.

EXAMPLE 8. Compounds 31, 32, and 35 inhibits LPS induced production of inflammatory mediator (TNF-α) in mouse bone marrow derived macrophages.

FIG. 20: Bone marrow derived mouse macrophages were treated with 100 μM of the test compounds for 8 hours. Pro-inflammatory cytokines such as TNF-α protein level was measured by real-time RT-PCR. LPS treatment (10 ng/ml) was used as positive control.

EXAMPLE 9. Compounds inhibits HMGB1 induced production of inflammatory mediators (TNF-α, i-NOS) and upregulate M2 biomarker CXCR4 in mouse macrophages:

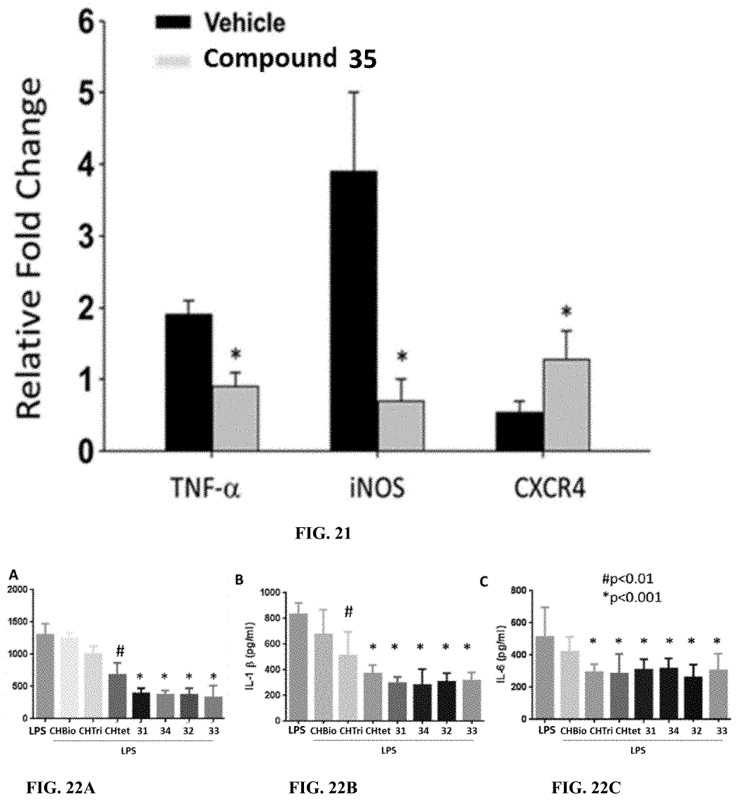

FIG. 21: The expressions of TNF-α and iNOS were both inhibited by compound 35 in macrophages. Interestingly CXCR4, an M2 macrophage marker, was upregulated by 35, suggesting potential effects on microphage polarization suggesting immune modulating activity. Bone derived macrophages from mouse were treated with HMGB1 for 8 hours, with or without 100 μM of compound 35. The mRNA levels of TNF-α, iNOS and CXCR4 were measured by real-time RT-PCR, and normalized to the expression level in control cells.

EXAMPLE 10. Compounds of present invention produces anti-inflammatory cytokines IL-10.

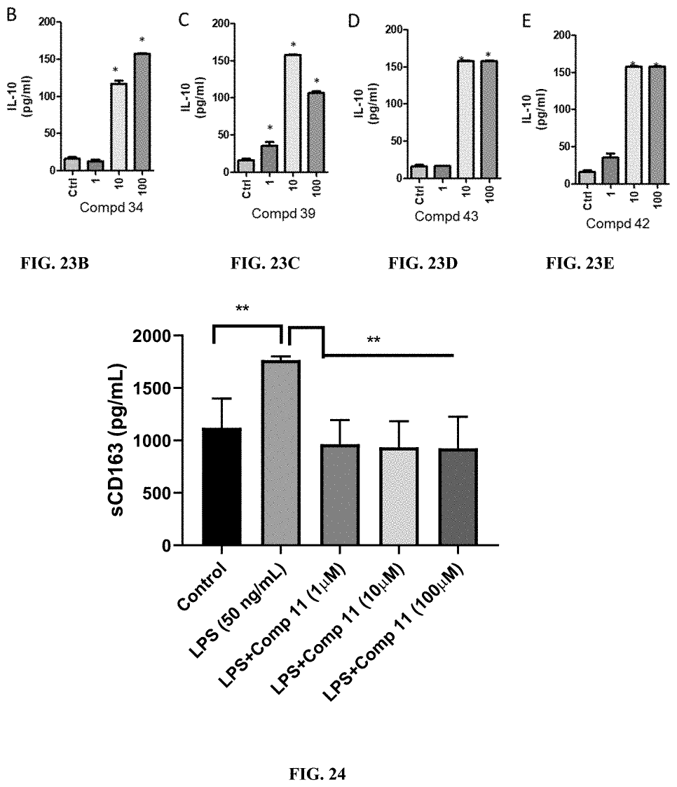

FIGS. 23A to 23E: Interleukin 10 (IL-10) is a cytokine with potent anti-inflammatory properties that plays a central role in limiting host immune response to pathogens, thereby preventing damage to the host and maintaining normal tissue homeostasis. Dysregulation of IL-10 is associated with enhanced immunopathology in response to infection as well as increased risk for development of many autoimmune diseases. Here we evaluated the compounds of present invention in upregulating the IL-10 levels in PBMC using ELISA assay. Briefly, 0.5 million cells were grown in RPMI with 10% FBS O/N in two 24 well plate. Next day, the media was taken out without disturbing the lower layer and different concentration of compounds (0, 1, 10, 100 μM) were added to make total amount of 0.5 ml volume with RPMI which was incubated for 48 hrs. Cells were harvested and the collected soup was analyzed using ELISA kit following manufacturer's instruction (Raybiotech). Compounds 11 increase IL-10 (FIG. 23A) after 24 h treatment to PBMC supernatants. FIGS. 23 B-E showed the increase in IL-10 with other analogs 34, 39, 43 and 42 when treated for 48 h and ELISA measurement done with cell lysates.

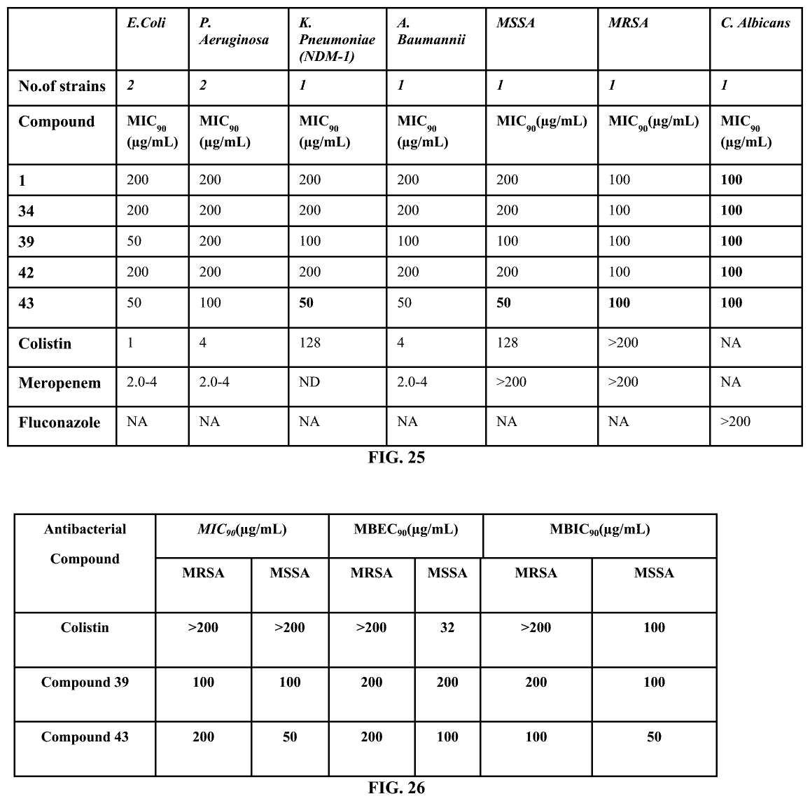

EXAMPLE 11. FIG. 25: Compounds of present invention decreases the LPS induced soluble CD163 (sCD163) in cell supernatants after 24 h as measured by ELISA showing decreased inflammation. FIG. 24 demonstrates that compounds of present invention decrease LPS induced sCD163 level in human blood cells.

EXAMPLE 12. Chitohexaose (Compound 31) protected mouse from lethal gram-negative sepsis against E. coli.

In an in vivo mice model, chitohexaose (Compound 31) protected mouse from lethal gram-negative sepsis against E. coli. Previously reported bacterial sepsis model [Roger et al., Proc Natl Acad Sci USA. 2009 Feb. 17; 106(7):2348-52] was recruited to study the efficacy of Compound 1 (FIG. 14). Intraperitoneally 2×10⁵ CFU of E. coli (ATCC-25922) was injected into BALB/c mice with and without Compound 31 (250 μg/animal). E. coli mediated sepsis induced mortality whereas simultaneous treatment of mice with Compound 31 protected (40%) from sepsis induced death. The above result explained that bacteria induced sepsis and mortality is inhibited and delayed for a certain period of time which may provide a window for therapy.

EXAMPLE 13. Compound 34, 39, 42 and 43 protected mice from CLP induced polymicrobial infection and sepsis.

To prove the concept and demonstrate the feasibility, we have tested compound 34 in mouse model of CLP. The CLP model consists of perforation of the cecum allowing the release of fecal material into the peritoneal cavity to generate an exacerbated immune response induced by polymicrobial infection. This model fulfills the human condition that is clinically relevant. Previously reported CLP sepsis protocol (Toscano et al, Journal of Visualized Experiments: 2011, (51), 2860] was recruited to study the efficacy of compound 34. Compound 34 (10 mg/kg) was injected intravenously into C57BL/6 mice (Jackson Laboratories, 10-12 weeks, N=15) into CLP group and CLP plus saline (0.5%) and antibiotics (primaxin, 5 mg/kg) after 16, 40 h post-surgery. As a result, CLP mediated sepsis induced organ dysfunction and death whereas simultaneous treatment of mice with compound 34 protected (˜93%) mice from sepsis induced death (14/15) as shown in FIG. 2. We have also demonstrated that in combination with the standard point of care anti-biotics primaxin (5 mg/kg), compound 34 protected (˜93%) mice from sepsis induced death (14/15) and simultaneously delayed the clinical symptoms such as body temperature lowering, shivering, huddling, loss of appetite, decreased movement, higher heart and respiratory rate.

Compound 39 and 43 (10 mg/kg/dose) while showed ˜60% protection, compound 42 (10.0 mg/kg/dose) demonstrated 80% protection to the mice from sepsis induced death as shown in FIGS. 15A-15D. It was also demonstrated that in combination with the standard point of care anti-biotics primaxin (5 mg/kg), the compounds protected (˜60-80%) mice from sepsis induced death and simultaneously delayed the clinical symptoms such as body temperature lowering, shivering, huddling, loss of appetite, decreased movement, higher heart and respiratory rate.

EXAMPLE 14. Histopathology score of organ tissues post-CLP mice.

Hematoxylin Eosin (H/E) staining of different organs of Sham, CLP and compounds 34 or 42 treated mice were done. Briefly, after tissues were collected, they were fixed in 10% buffered neutral formalin, processed, embedded in paraffin and sectioned at 4μ for routine hematoxylin-eosin staining. CLP mice showed micro thrombi and congestion in the heart, lungs, liver, kidney and brain, increased germinal centers size in spleen, necrosis of villi in gut and loss of testicular epithelium. On treatment with compound 34, and 42 all these changes were reversed to a major extent and tissues resembled to sham group which was shown here as injury score (FIGS. 16 A-C).

EXAMPLE 15. Biomarker study of the post-CLP serum.

FIG. 17: The plasma collected from tail vein 48 h post-surgery were stored at −30° C. and were analyzed for TNF-α, IL-6, and IL-1β levels for the Sham, CLP, CLP+compound 34, CLP+primaxin, CLP+primaxin+compound 34 and control (saline injected) groups. Compound 34 alone or in combination with antibiotics primaxin decreased the level of TNF-α, IL-1β and IL-6 statistically significant (n=4, p<0.0005) in compared to CLP group of mice. Briefly commercially available ELISA Kit was used to estimate the above cytokines by a sandwich ELISA method. The ELISA plates were coated with capture antibodies followed by incubation with test samples and appropriate standards. Then it was probed with biotin labeled secondary antibodies and avidin-peroxidase. Color was developed using TMB and optical density (OD) was recorded.

EXAMPLE 16. Compounds of present invention have broad-spectrum antimicrobial activity.

The compounds were screened against selected gram negative (E. coli, P. aeruginosa, A. baumannii, K. pneumonia), gram positive (MRSA) as well as fungus (C. albicans) mostly found in burn and septic wounds. Most of them showed antimicrobial activity with MIC₉₀ of 50-200 mg/L (FIG. 25). All prepared compounds have been evaluated in a Minimum Inhibitory Concentrations (MIC) assay with both test and control articles in accordance with guidelines of the Clinical Laboratory Standards Institute (CLSI) for broth microdilution susceptibility testing. Briefly, test and control compounds have been dissolved in DMSO, diluted to proper concentrations and added to 96-well microdilution trays. Brain Heart Infusion Broth (BHI) was used for studies with bacterial strains such as S. aureus, E. coli, P. aeruginosa, K. pneumoniae, A. baumannii and Candida albicans. Compounds were diluted serially from 200 μg/ml to 0.0625 μg/ml and plated in 96 well plates, and inoculated with approximately 1×10⁵ CFU of each organism. The MIC endpoint was determined for each compound after 24 hrs as the lowest concentration of test or control compound which completely inhibits growth of the organism in microdilution.

EXAMPLE 17. Compounds of present invention inhibited biofilm formation.

Based on the in vitro antimicrobial MIC data, we have selected three compounds 31, AVR-45 (compound 39) and compound 43 for further study against MRSA. All three compounds demonstrated better activity against MRSA and MSSA strains compared to colistin (standard of care antibiotic) which was used as positive control (FIG. 26). Further we studied their biofilm inhibition and eradication activities against biofilm cells as described previously [Ceri et al, Journal of Clinical Microbiology 1999, 37, 1771-1776]. The minimum biofilm eradication concentrations (MBIC) of compound 39 were superior to colistin for MRSA. Thus, these compounds with activities against S. aureus biofilms will have significant impact on controlling recalcitrant biofilm-mediated endovascular infections [Li et al, J Infect Dis. 2016 Nov. 1; 214(9):1421-1429; Archer et al, Virulence 2011, 2, 445-459]. Briefly, we used Calgary Biofilm Device (CBD) technology for the biofilm susceptibilities to compounds. The CBD produces 96 equivalent biofilms for the assay of antibiotic susceptibilities by the standard 96-well technology. Susceptibility to a standard group of compounds and antibiotics was determined for National Committee for Clinical Laboratory Standards (NCCLS) as described previously.

EXAMPLE 18. The broad-spectrum antimicrobial activity of the compounds of present invention is via disruption of cell membrane.

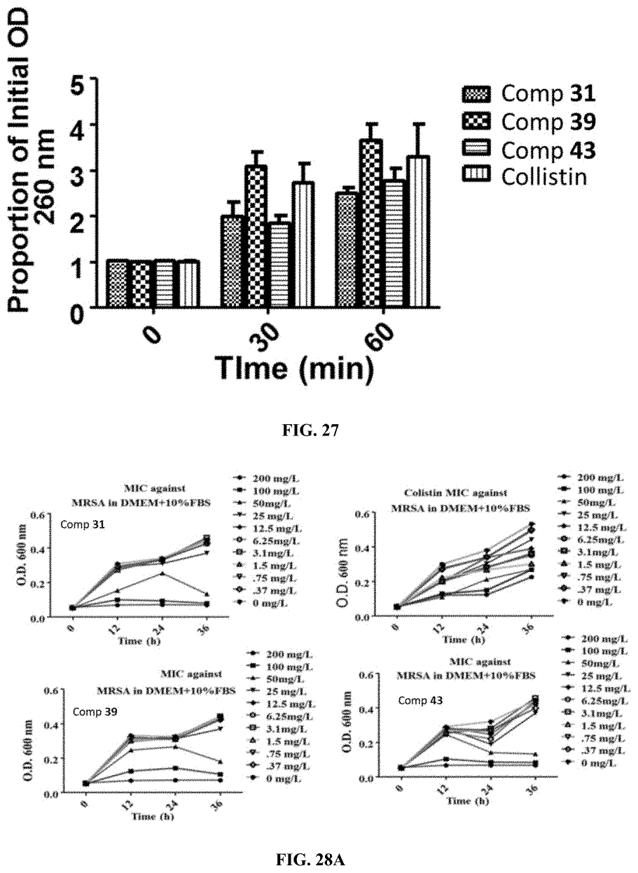

The effects of the AVR compounds on exponentially growing MRSA and membrane integrity were evaluated. MRSA re-suspended in phosphate-buffered saline was exposed to antibiotics as well as AVR compounds 31, 39 and 43 at their MIC value in brain heart infusion broth for 30 min and 1 hour before measuring the absorbance of leaked cellular material detected at an optical density of 260 nm in the culture filtrate (FIG. 27).

EXAMPLE 19. Compounds of present invention do not bind to the plasma serum protein.

One of the major difficulties in designing systemic, oral or topical drug candidate is their poor plasma/tissue bioavailability due to binding of the drug to the plasma serum protein. So, we have studied the effect of serum on compounds activities by microdilution MICs in DMEM containing 10% bovine serum (GIBCO, MA) [Hurdle et al, Journal of Antimicrobial Chemotherapy 2008, 62, 1037-1045]. The results showed (FIG. 28) that all compounds were active against MRSA and didn't bind to the serum protein suggesting them to have good bioavailability in the target tissue.

EXAMPLE 20. Compounds of present invention are not toxic to fibroblast cells.

Cytotoxicity and therapeutic index were also evaluated by exposing to NIH 3T3 mouse fibroblast cell lines (ATCC, Manassas, Va.) to antibiotic colistin and AVR compounds for 24 h, followed by an MTT [3-(4,5-dimethyl-2-thiazolyl)-2,5-diphenyl-2H-tetrazolium bromide, Thermo Fischer, MA] assay as described previously. The cytotoxicity for all the AVR compounds in NIH 3T3 mouse fibroblast cell line is shown in FIG. 25, indicating that AVR compounds selectively inhibit the growth of MRSA without compromising the growth of fibroblast cells which are critical in wound healing process and has >4 fold therapeutic index for compounds 39 and 43.

EXAMPLE 21. Compounds 31 and 32 decreased VEGF production in ARPE-19 cell.

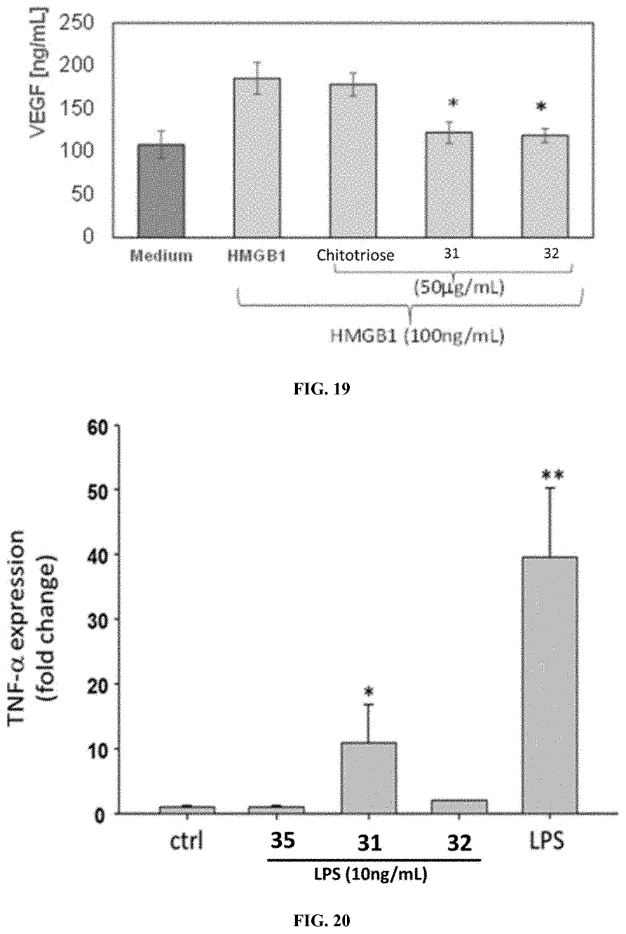

Central to photoreceptor survival and function, the RPE is the major source of the angiogenic factor VEGF and therefore plays a central role in the modulation and progression of choroidal neovascularization [Spilsbury et al, The American Journal of Pathology 2000, 157, 135-144; Betts et al, ISRN Ophthalmology 2011, 2011, 184295] leading to AMD. As part of our preliminary result to evaluate if compounds 31 and 32 can inhibit the HMGB1 (an endogenous ligand for TLR4) induced VEGF production in ARPE-19 cells. FIG. 19 showed that compounds 31 and 32 (50 μg/mL) effectively reduced the HMGB1 induced VEGF production in ARPE-19 cells in a statistically significant manner (p<0.01). 2×10⁵ ARPE-19 cells were seeded in 24 well plate for 24 h in full medium containing 10% serum following which they were maintained for additional 24 h with serum free medium. Cells were treated with 0 μg/mL (medium) or 100 ng/mL of HMGB1 along with or without of 50 μg/mL of test compounds for 24 h. Supernatant was collected and assayed using human VEGF ELISA kit from Peprotech according to manufacturer's instructions. RPE cells are located adjacent to choroidal capillaries and other major ocular vasculatures. Thus, these finding suggests that compounds 31 and 32 may have a significant effect on the inhibition of angiogenesis (via inhibiting inflammation and decreasing VEGF) of choroidal as well as retinal capillaries, which contribute to the development of AMD and retinopathy.

EXAMPLE 22. Compound 32 decreased the choroidal neovascularization ˜60% as compared to the positive control in a lased induced CNV mouse model for wet AMD.

To demonstrate the in vivo angiostatic effects of the AVR compounds, we have tested the compounds in mouse model of laser-induced CNV (FIG. 18). Laser CNV was induced in C57BL/6 (10-12 weeks) mice using an Iridex Oculight GL 532 nm diode laser (Mountain View, Calif.) connected to the Micron IV fundus imaging system using a laser injector (Phoenix Research Laboratories, Pleasanton, Calif.). The parameters used to reproducibly obtain successful laser spots (as confirmed by a gas bubble formation indicating rupture of Bruch membrane) were: 350 mW, 75 msec, and 50 μm spot size. Four laser spots were applied; 2-3 disc diameters from the optic nerve. Mice were treated with either PBS (negative control), Compound 32, and chitotriose or a positive control R84 (anti-VEGF antibody) on days 2, 4 and 6 after laser (n=4-6 mice/group). Compounds 32, and chitotriose and BSS (vehicle) were administered by IP injection once daily, and was started one day before laser and continued for 10 days after laser. By the end of the experiment, mouse eyes were examined by fundus fluorescein angiography and/or optical coherence tomography (OCT) to visualize the CNV lesions. Afterwards animals were sacrificed, and RPE/choroid/sclera flat mounts were prepared and stained with both FITC-conjugated isolectin B4 and anti-ICAM-2 antibody to quantitatively measure the size of CNV. We have performed two experiments to test the effects of Compounds 32, and chitotriose on laser-induced CNV. As shown in FIG. 18, 200 μg daily i.p. injection of compound 32 was able to reduce the average size of CNV lesions to about 60% of those in control mice treated with vehicle only (balanced salt buffer) and comparable to the positive control.

EXAMPLE-23. Synthesis of compounds 1, 4, 7 and 42.

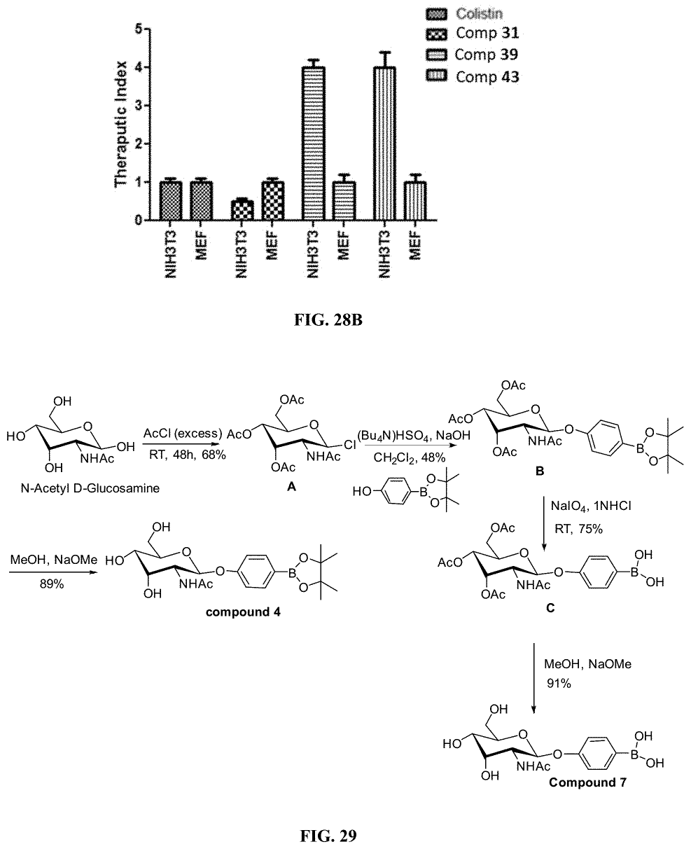

Synthesis of compound 4 (FIG. 29). 2-Acetamido-3,4,6-tri-O-acetyl-2-deoxy-α-D-glucopyranosyl chloride A: To a stirred solution of commercial 2-acetamido-2-deoxy-β-D-glucose (25 g, 0.113 mmol), acetyl chloride (50 mL) was added and the reaction mixture was stirred overnight. The reaction mixture was diluted with further portion of CH₂Cl₂ (100 mL) and poured into water-ice mixture. The organic phase was extracted with ice-cold saturated aqueous solution of NaHCO₃ (2×150 mL) and with ice-cold water (150 mL). The organic phase was dried, concentrated and run over a short column using EtOAc (15-50%) in hexane as an eluent to give pure product A in 68% of yield as a solid product. The NMR spectra were in compliance with literature reported (Sauerzapfe, Namdjou et al. 2008).

p-Pinacoloneboronatephenyl 2-acetamido-3,4,6-tri-O-acetyl-2-deoxy-β-D-glucopyranoside B: A solution of 2-acetamido-3,4,6-tri-O-acetyl-2-deoxy-β-D-glucopyranosyl chloride A (5.0 g, 13.67 mmol), tetrabutylammonium hydrogen sulfate (4.65 g, 13.67 mmol), and 4-(4, 4,5,5-tetramethyl-1,3,2-dioxaborolan-2-yl)phenol (3.15 g, 1.05 equiv.) in a mixture of CH₂Cl₂ (100 mL) and 1N NaOH (50 mL) was stirred vigorously for 1 h. The mixture was extracted with CH₂Cl₂ (2×125 mL), and washed with water and brine. The combined organic phase was dried over anhydrous Na₂SO₄, filtered, concentrated and purified by silica-gel column using EtOAc (15-50%) in hexane as an eluent to give pure product B in 48% of yield as a solid product.

p-Pinacoloneboronatephenyl 2-acetamido-2-deoxy-β-D-glucopyranoside 4: The above p-Pinacoloneboronatephenyl 2-acetamido-3,4,6-tri-O-acetyl-2-deoxy-β-D-glucopyranoside B (2.45 g, 4.45 mmol) was suspended in dry MeOH (70 mL) and methanolic solution of NaOMe (1M solution, 4.25 equivalent) was added and the mixture was stirred at RT until dissolution was complete (15 min). Dowex 50WX2-200 (5 g, previously washed in methanol) was added and removed by filtration after 15 min. The solution was evaporated in vacuo to dryness. White solid was dissolved in CH₂Cl₂ and MTBE was added to precipitate out white solid. The solid was filtered off and dried over high vacuum to provide compound 4 in 89% of yield. LC/MS=423.9 (M+1); ¹H NMR (CD₃OD, 500 MHz): δ 1.41 (s, 12H), 2.01 (s, 3H), 3.41-3.45 (m, 1H), 3.58-3.64 (m, 2H), 3.68-3.80 (m, 1H), 3.90-4.15 (m, 2H), 5.19 (d, 1H), 7.10 (d, 2H), 7.65 (d, 2H).

p-Boronic acid phenyl 2-acetamido-2-deoxy-β-D-glucopyranoside 7: The above compound p-pinacoloneboronatephenyl 2-acetamido-3,4,6-tri-O-acetyl-2-deoxy-β-D-glucopyranoside B (1.5 g, 2.73 mmol) in acetone/H₂O (4:1) was treated with NaIO₄ (2.5 equiv.), NH₄OAc (1.5 equiv) and was stirred at RT for 24 hrs. pH of the reaction mixture was adjusted to 3 by adding 1N HCl and stirred for additional 30 mins. The reaction mixture was extracted with DCM and washed with brine, dried over anhydrous Na₂SO₄, filtered off. The combine filtrate was concentrated to give crude boronic acid and purified by silica-gel column using EtOAc (0-50%) in hexane as an eluent to give product C in 75% yield as a solid product. This product was then treated with NaOMe in accordance with the procedure above for 4, to give compound 7 in 91% of yield as a white solid. The NMR and MS spectra confirmed the structure of the above product. ¹H NMR (D₂O+DMSOd₆, 500 MHz): δ 1.21 (s, 3H), 2.6-2.8 (m, 4H), 3.15 (m, 2H), 3.68-3.80 (m, 1H), 4.35 (m, 1H), 6.25 (d, 2H), 7.01 (d, 2H).

Synthesis of compound 1. Following the same procedure as described for compound 4, the intermediate (0.25 g, 0.53 mmol) was converted to compound 1 in 92% of yield as white solid. The NMR and MS spectra confirmed the structure of the above product. LC/MS=339.9 (M+1); ¹H NMR (CD₃OD, 500 MHz): δ 2.01 (s, 3H), 3.41-3.45 (m, 1H), 3.58-3.64 (m, 2H), 3.68-3.80 (m, 1H), 3.90-4.15 (m, 2H), 5.25 (d, 1H), 7.21 (d, 1H), 7.32 (s, 1H), 7.83 (s, 1H).

Synthesis of compound 42 (FIG. 30). p-Nitrophenyl 2-acetamido-3,4,6-tri-O-acetyl-2-deoxy-β-D-glucopyranoside intermediate D: Following the procedure for compound 4, compound 2-acetamido-3,4,6-tri-O-acetyl-2-deoxy-β-D-glucopyranosyl chloride A (1.0 g, 2.73 mmol) was coupled with commercial p-nitro-phenol (1.05 equiv.) to give intermediate D in 89% of yield as a white solid. ¹H NMR

p-Nitrophenyl 2-acetamido-2-deoxy-β-D-glucopyranoside 42: Following the same procedure as used for synthesizing 4, the above intermediate D (0.25 g, 0.53 mmol) was converted to compound 42 in 92% of yield as pure white solid. LC/MS=343.1 (M+1); ¹H NMR (D₂O)+CD₃OD, 500 MHz): δ 2.01 (s, 3H), 3.46 (t, 1H), 3.60-3.65 (m, 2H), 3.68-3.81 (dd, 1H), 3.90-4.15 (m, 2H), 5.24 (d, 2H), 7.20 (d, 2H), 8.23 (d, 2H).

Synthesis of compound 11 (FIG. 31): To a stirred solution of compound 42 (2.0 g, 5.84 mmol) in anhydrous CH₂Cl₂ triethylamine (1.5 equivalent) was added followed by drop wise addition of MsCl (1.0 equivalent) and stirred for overnight, reaction completion noted by TLC that provided intermediate 2, in 89% of yield after column purification. This product was directly used in the next step without any further spectral analysis. To the O-Mesyl derivative E (1 equivalent) in anhydrous DMF was added NaN₃ (3 equivalent) and heated at 65° C. for 6 h to give intermediate F in 82% as a pure enough product determined by TLC to proceed next without any purification. The azide intermediate F was converted to intermediate amine G by using standard azide to amine reduction procedure using TPP, H₂O by stirring at 23° C. overnight. This intermediate product G was then coupled with commercially available NH-succinamide-Biotin using 1.5 equivalents of EDCI, DIPEA and catalytic amount of HOBt to give 91% yield of compound 11 after column purification. The structure of compound 11 was confirmed by both ¹HNMR and MS.

¹H NMR (DMSOd₆) of compound 11: δ 1.25 (m, 2H), 1.55-1.58 (m, 4H), 1.88 (s, 3H), 2.13 (t, 2H), 2.42-2.59 (dd, 2H), 2.73-2.84 (dd, 1H), 3.12-3.18 (m, 2H), 3.20-3.25 (m, 1H), 3.72-3.78 (m, 1H), 4.14 (m, 1H), 4.28 (m, 1H), 5.15 (d, 1H), 6.38-6.43 (d, 1H), 7.13-7.16 (d, 2H), 7.91-7.99 (m, 2H), 8.16-8.19 (d, 2H). LC-TOF (+ESI): 568 (M+).

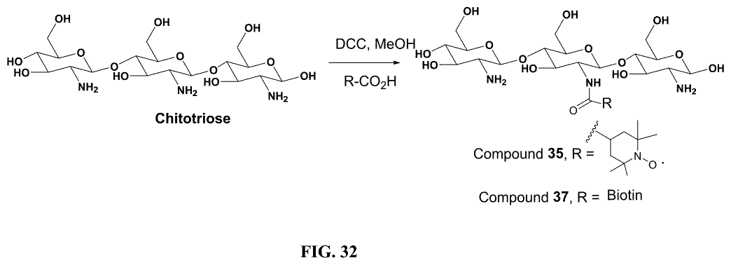

Synthesis of compound 35 and 37 (FIG. 32): To chitotriose (10 mg, 0.015 mmol) in 5 mL of methanol, a methanolic solution of 4-carboxy-TEMPO (4.5 mg, 0.026 mmol) was added. To this a methanolic solution of DCC (4.66 mg, 0.026 mmol) and cat DMAP was added and stirred at room temperature for 48 h, chilled at 2° C. for 12 h. Ether was added to precipitate white solid, filtered and dried to provide 4.0 mg of white powder. LC/MS=684 (M+1); ¹H NMR (DMSO-D₆, 500 MHz): δ 1.15 (s, 12H), 1.35-1.55 (m, 4H), 1.97-2.05 (bs, 10H), 2.43 (m, 1H), 2.80-3.23 (m, 4H), 3.25-3.66 (m, 11H), 4.07 (s, 2H), 4.41 (s, 2H), 4.65 (s, 2H), 5.18 (d, 1H), 5.52 (bs, 2H), 8.15 (d, 1H, NH).

Synthesis of compounds 45-48: To a stirred suspension of D-glucosamine (100 g, 0.55 mol) in EtOH (500 mL), NaOEt (30 g, 0.55 mol) was added. After 10 minutes, the mixture was treated with dimethylmaleic anhydride (0.5 eq) and stirred for 20 minutes. Triethylamine (65.2 mL, 0.465 mol) was added and the reaction mixture was again treated with remaining dimethylmaleic anhydride (0.5 eq). The reaction mixture was warmed to 60° C. with stirring for 2 h, EtOH was evaporated and dried. The residue was treated with pyridine, acetic anhydride and stirred at room temp for 20 h. The reaction was monitored by TLC, the solvent was evaporated and the residue poured in to ice, extracted with chloroform (3×1 L), washed with aqueous hydrochloric acid (3%) 1 L, saturated sodium bicarbonate solution (1 L), distilled water (1 L), dried with anhydrous sodium sulfate. The residue was purified by silica gel chromatography using EtOAc (20-30%) in pet-ether as eluent to get the tetraacetate compound (80 g, ˜37%). ¹H NMR (400 MHz, CDCl₃): δ 1.91 (3H, s), 1.94 (6H, s), 2.01 (s, 3H), 2.03 (s, 3H), 2.09 (s, 3H), 3.92-3.96 (m, 1H), 4.0-4.12 (dd, 1H), 4.18-4.23 (dd, 1H), 4.30-4.33 (dd, 1H, J=4.4 Hz), 5.14 (t, 1H, J=9.2 Hz), 5.69 (t, 1H, J=9.2 Hz), 6.34 (d, 1H, J=8.8 Hz).

To a solution of tetraacetate compound (100 g, 0.219 mol) in DMF, slowly added hydrazine hydrate (12 ml, 0.219 mol) at 23° C., stirred same temp for 5-6 h, and monitored by TLC. The reaction mixture was diluted with ethyl acetate (2 L) and washed with water (3×1 L), brine (1 L) and dried over Na₂SO₄ and evaporated to get anomeric alcohol compound (65 g, ˜71%). ¹H NMR (400 MHz, CDCl₃): δ 1.85 (s, 3H), 1.90 (s, 6H), 1.98 (s, 3H), 2.05 (s, 3H), 3.79-3.84 (m, 1H), 3.95-4.06 (dd, 1H), 4.10-4.13 (dd, 1H), 4.20-4.24 (dd, 1H), 5.06 (t, 1H, J=9.6 Hz), 5.42 (d, 1H, J=8.4 Hz), 5.60 (t, 1H, J=9.6 Hz).

To a stirred solution of anomeric alcohol compound (35 g, 0.084 mol) and imidazole (14.4 g, 0.211 mol) in DCM, was added TBDMSCl (15.2 g, 0.101 mol) portion wise at 23° C. and stirred at same temp for 16 h, monitored by TLC, diluted with DCM (1 L), washed with water (2×1 L), brine (500 mL), dried over Na₂SO₄ and concentrated to get crude. The residue was purified by silica gel chromatography using EtOAc (15-20%) in pet-ether as eluent to afford O-TBDMS compound (27 g, ˜61%). ¹H NMR (400 MHz, CDCl₃): δ 0.01 (s, 3H), 0.05 (s, 3H), 0.76 (s, 9H), 1.91 (s, 3H), 1.93 (s, 6H), 2.01 (s, 3H), 2.07 (s, 3H), 3.80-3.83 (m, 1H), 3.98-4.03 (dd, 1H), 4.10-4.14 (dd, 1H), 4.20-4.24 (dd, 1H), 5.05 (t, 1H, J=9.6 Hz), 5.36 (d, 1H, J=8.4 Hz), 5.66 (t, 1H, J=9.6 Hz).

To a stirred solution of compound O-TBDMS compound (27 g, 0.051 mol) in MeOH (100 mL), was added NaOMe (2.76 g, 0.052 mol) portion wise at 23° C. and stirred at same temp for 3-4 h. The reaction was monitored by TLC, MeOH was concentrated under reduced pressure and diluted with 50 mL water, pH was adjusted to 6.5-7.0, the solid was filtered and dried to get pure compound deacetylated compound (16 g, ˜77%). H NMR (400 MHz, CDCl₃): δ 0.03 (s, 3H), 0.05 (s, 3H), 0.76 (s, 9H), 1.94 (s, 6H), 3.45-3.47 (m, 1H), 3.57-3.61 (m, 1H), 3.77-3.90 (m, 3H), 4.21 (t, 1H, J=8.0 Hz), 5.23 (d, 1H, J=8.0 Hz).

A suspension of compound deacetylated compound (30 g, 0.074 mol) and dibutyltin oxide (37.25 g, 0.149 mol) in toluene (400 mL) was heated under reflux for 12 h, tetrabutylammonium iodide (55.2 g, 0.149 mol) and benzyl bromide (25.5 g, 0.149 mol) were added and the mixture was gently refluxed for 3 h, the reaction mixture was cooled, concentrated to get crude. The residue was purified by silica gel chromatography by using 15-20% EtOAc in pet-ether to yield O-dibenzylated compound (25 g, ˜60%). ¹H NMR (400 MHz, CDCl₃): δ 0.06 (s, 3H), 0.02 (s, 3H), 0.73 (s, 9H), 1.83-1.93 (bs, 6H), 3.56-3.61 (m, 1H), 3.72-3.80 (m, 2H), 3.86-3.89 (dd, 1H), 4.09-4.14 (dd, 1H), 4.53 (d, 1H, J=12 Hz), 4.56-4.63 (dd, 2H), 4.69-4.76 (dd, 2H), 5.16 (t, 1H, J=8.0 Hz), 7.15-7.24 (m, 5H), 7.33-7.37 (m, 5H).

To a mixture of compound anomeric alcohol compound (5 g, 0.012 mol) and CCl₃CN (2.06 g, 0.014 mol) in dry CH₂Cl₂ was added DBU (0.37 g, 0.002 mol) and stirred at RT for 15-16 h. The reaction mixture was concentrated to get crude. The crude was purified by silica gel chromatography by using EtOAc (25-35%) in pet-ether to yield —OTCA compound (3.8 g, 55%). ¹H NMR (400 MHz, CDCl₃): δ 1.91 (s, 3H), 1.93 (s, 6H), 2.01 (s, 3H), 2.07 (s, 3H), 3.93-4.01 (m, 1H), 3.98-4.03 (dd, 1H), 4.33-4.40 (m, 2H), 5.20 (t, 1H, J=9.2 Hz), 5.73 (t, 1H, J=9.2 Hz), 6.45 (d, 1H, J=9.2 Hz), 8.67 (s, 1H).

A mixture of —OTCA compound (4 g, 0.007 mol) and the O-dibenzyl compound (3.3 g, 0.0057 mol) is taken in oven dried round bottom flask containing activated molecular sieves powder (4 A°). Then the RB was back filled with argon twice and added dry DCM (10 mL), stirred at 23° C. for 2 h. Then resulting reaction mixture was cooled to −10° C. and added 0.1M solution of TfOH in DCM and further stirred for 16 h. The reaction mixture was concentrated under reduced pressure to get crude mixture which was purified by silica gel chromatography by using EtOAc (25-35%) in pet-ether to yield the —OAc—OBn protected disaccharide (2 g, 30%). ¹H NMR (400 MHz, CDCl₃): δ −0.005 (s, 3H), −0.11 (s, 3H), 0.71 (s, 9H), 1.76 (bs, 6H), 1.90 (s, 3H), 1.95 (bs, 6H), 1.98 (s, 3H), 3.36-3.44 (m, 3H), 3.48-3.55 (m, 3H), 3.80-3.83 (m, 2H), 3.90-3.93 (dd, 1H), 4.04-4.12 (m, 3H), 4.14-4.20 (dd, 1H), 4.42 (d, 1H, J=12.4 Hz), 4.57-4.64 (dd, 2H), 4.81 (d, 1H, J=12.4 Hz), 5.03-5.08 (m, 2H), 5.36 (d, 1H, J=8.4 Hz), 5.61 (t, 1H, J=9.2 Hz), 7.13-7.19 (m, 5H), 7.33-7.39 (m, 5H).

To a solution of compound —OAc—OBn protected disaccharide (5.5 g, 0.0056 mol) in dry THE (50 mL) was added AcOH (0.36 mL, 0.0063 mol) and cooled to −5° C. Then added 1 M TBAF (6.3 mL, 0.0063 mol) soln in THE at −5° C. stirred at room temperature for 16 h. After completion of the reaction, reaction mixture was quenched with sat NaCl solution, extracted with DCM and concentrated under reduced pressure to obtain crude TBDMS deported compound (3 g, crude) which was used further reaction without purification. ¹H NMR (400 MHz, CDCl₃): δ 1.76 (s, 6H), 1.89 (s, 3H), 1.95 (bs, 6H), 1.98 (s, 3H), 3.38-3.55 (m, 3H), 3.62-3.66 (m, 1H), 3.76-3.80 (m, 1H), 3.90-3.94 (m, 1H), 4.06-4.22 (m, 6H), 4.14-4.20 (dd, 1H), 4.40 (d, 1H, J=12.8 Hz), 4.57-4.64 (dd, 2H), 4.85 (d, 1H, J=12.8 Hz), 5.01-5.07 (m, 2H), 5.33 (d, 1H, J=8.4 Hz), 5.55-5.63 (t, 1H, J=9.2 Hz), 7.11-7.20 (m, 5H), 7.29-7.42 (m, 5H).

To a mixture of compound TBDMS deported anomeric —OH compound (4 g, 0.0045 mol) and CCl₃CN (0.54 mL, 0.0054 mol) in dry CH₂C₂ was added DBU (0.13 mL, 0.0009 mol) stirred at RT for 15-16 h. The reaction mixture was concentrated to get crude. The crude was purified by silica gel chromatography by using EtOAc (25-35%) in pet-ether to yield the —OTCA compound (1 g, 20%).

A mixture of the —OTCA compound (1 g, 0.89 mmol) and R—OH (0.7 eq) is taken in oven dried round bottom flask containing activated molecular sieves powder (4 A°) then RB was back filled with argon twice and added dry DCM (20 mL), stirred at rt for 2 h. Then resulting reaction mixture was cooled to −10° C. and added 0.1M solution of TfOH (1.3 mL, 0.13 mmol) in DCM and further stirred for 16 h. The reaction mixture was concentrated under reduced pressure to get crude mixture. The crude was purified by silica gel chromatography by using EtOAc (25-35%) in pet-ether to yield the —OAC-NDMM protected precursor compounds of 45A-48A (˜40-45%).

Compound-45A: ¹H NMR (400 MHz, CDCl₃): δ 1.76 (s, 6H), 1.89 (s, 3H), 1.95 (bs, 6H), 1.98 (s, 3H), 3.49-3.51 (m, 2H), 3.64-3.71 (m, 2H), 3.94-3.96 (m, 1H), 4.15-4.19 (m, 6H), 4.40 (m, 1H), 4.56-4.65 (m, 2H), 4.86-4.91 (m, 1H), 5.06-5.09 (m, 1H), 5.33 (d, 1H, J=8.4 Hz), 5.55-5.63 (t, 1H, J=9.2 Hz), 6.91 (d, 2H, J=8.4 Hz), 7.12-7.21 (m, 5H), 7.29-7.42 (m, 5H), 8.06 (d, 2H, J=8.4 Hz). LC/MS: M+=982.

Compound-46A: ¹H NMR (400 MHz, CDCl₃): δ 1.21-1.48 (m, 10H), 1.76 (bs, 6H), 1.89 (s, 3H), 1.95-1.96 (s, 9H), 1.98 (s, 3H), 3.36-3.38 (m, 1H), 3.43-3.46 (m, 3H), 3.59 (d, 1H, J=8.4 Hz), 3.89-3.92 (m, 2H), 4.04-4.08 (m, 4H), 4.41 (d, 1H, J=10.0 Hz), 4.60 (s, 2H), 4.84 (d, 1H, J=10.0 Hz), 4.93 (d, 1H, J=6.4 Hz), 5.04 (t, 1H, J=7.2 Hz), 5.35 (d, 1H, J=6.4 Hz), 5.57-5.61 (t, 1H, J=7.2 Hz), 7.13-7.19 (m, 5H), 7.28-7.40 (m, 5H). LC/MS: M⁺-2=943.

Compound-47A: ¹H NMR (400 MHz, CDCl₃): δ 1.21-1.35 (m, 8H), 1.45-1.62 (bs, 8H), 1.76 (bs, 6H), 1.90 (s, 3H), 1.95-2.22 (m, 12H), 3.43-3.46 (m, 4H), 3.60-3.71 (m, 1H), 3.94 (d, 1H), 4.04-4.12 (m, 4H), 4.20 (m, 1H), 4.55-4.59 (m, 1H), 4.62-4.70 (m, 2H), 4.84 (d, 1H, J=10.0 Hz), 4.93 (d, 1H, J=6.4 Hz), 5.30 (t, 1H, J=7.2 Hz), 5.35 (m, 1H), 5.61 (t, 1H, J=7.2 Hz), 7.13-7.19 (m, 5H), 7.28-7.40 (m, 5H). LC/MS: M⁺-2=1015.

Compound-48A: ¹H NMR (400 MHz, CDCl₃): δ 1.76 (s, 6H), 1.90 (s, 3H), 1.95 (s, 3H), 1.98 (s, 6H), 2.01 (s, 3H), 3.43-3.49 (m, 3H), 3.62-3.64 (m, 1H), 3.72 (s, 3H), 3.91-3.94 (m, 1H), 4.07-4.21 (m, 5H), 4.42 (d, 1H, J=12.4 Hz), 4.59 (s, 2H), 4.85 (d, 1H, J=12.4 Hz), 5.05 (t, 1H, J=10.8 Hz), 5.34-5.37 (m, 2H), 5.60 (t, 1H, J=10.8 Hz), 6.69 (d, 2H, J=9.2 Hz), 6.77 (d, 2H, J=9.2 Hz), 7.11-7.21 (m, 5H), 7.29-7.42 (m, 5H). LC/MS: M+=986.

Compounds 45 was synthesized from compound 45A (0.5 g) by removal of NDMM group using hydrazine hydrate in HCl followed by treatment with Ac₂O to introduce NHAc group. Finally removal of —OAc groups by NaOCH₃/MeOH at room temperature as described previously [Mohamed R. E et al, Carbohydrate research, 2001, 331, 129-142] afforded compound 45 (22 mg white solid). ¹H NMR (400 MHz, CDCl₃): δ 1.98 (s, 6H), 3.43-3.49 (m, 3H), 3.62-3.64 (m, 1H), 3.72 (s, 3H), 3.91-3.94 (m, 1H), 4.23-4.42 (m, 3H), 4.62 (m, 4H), 5.90 (d, 1H, J=10.8 Hz), 6.42 (d, 1H, J=10.8 Hz), 6.91 (d, 2H, J=8.4 Hz), 7.12-7.21 (m, 5H), 7.29-7.42 (m, 5H), 8.06 (d, 2H, J=8.4 Hz). LC/MS: M+=710.

EXAMPLE-24. Topical/intravitreal formulation.

The table below represents exemplary ranges for a topical or intravitreal ophthalmic composition according to the present invention:

| Ingredients | w/v % |
|---|---|
| Compound 32 | 0.1 to 1.5 |
| Mannitol | 2.0 |
| Sodium acetate | 0.5 |
| Acetic acid | 0.02 |
| PEG 8000 | 2.0 |
| Polysorbate 80 | 1.0 |
| HPMC | 0.5 |
| Sodium hydroxide/Hydrochloric acid | For adjusting pH 6.5-7.4 |
| Water | Q.S. to 100 |

EXAMPLE-25. Injectable Formulation.

The table below represents exemplary ranges for an intravenous (IV) composition according to the present invention: The compound of the invention is dissolved in most of the water (35° 40° C.) and the pH adjusted to between 6.5 and 7.4 with the hydrochloric acid or the sodium hydroxide as appropriate. The batch is then made up to volume with water and filtered through a sterile micropore filter into a sterile 10 mL amber glass vial (type 1) and sealed with sterile closures and over seals.

| Ingredients | Amount | |
|---|---|---|
| Compound 1 or 4 or 42 | 5-10 | mg/kg |
| Hydrochloric Acid Solution 0.1M or | 4.0 to 7.0 | (pH) |
| Sodium Hydroxide Solution 0.1M q.s. | 4.0 to 7.0 | (pH) |
| to pH | | |
| Sterile water q.s. to | 10 | mL |

EXAMPLE-26. The tables below represents exemplary ranges for topical gel, lotion and spray compositions according to the present invention.

Oral Formulations. The composition of the present invention is typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the composition may be formulated to provide, e.g., maximum and/or consistent dosing for the particular form for oral, rectal, topical, intravenous injection or parenteral administration. While the composition may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins, 2000; Martindale, The Extra Pharmacopoeia, Thirty-Second Edition (The Pharmaceutical Press, London, 1999); Remington: The Science and Practice of Pharmacy, Pharmaceutical Press; 22nd Edition (2012); all of which are incorporated by reference, and the like, relevant portions incorporated herein by reference.

For example, the composition may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The composition may be administered in the form of liposome delivery systems, e.g., small unilamellar vesicles, large unilamallar vesicles, and multilamellar vesicles, whether charged or uncharged. Liposomes may include one or more: phospholipids (e.g., cholesterol), stearylamine and/or phosphatidylcholines, mixtures thereof, and the like.

The composition may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the composition may be coupled one or more biodegradable polymers to achieve controlled release of the composition, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the composition and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

For direct delivery to the nasal passages, sinuses, mouth, throat, esophagous, trachea, lungs and alveoli, the composition may also be delivered as an intranasal form via use of a suitable intranasal vehicle. For dermal and transdermal delivery, the composition may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of composition may include the following forms.

Capsules. Capsules may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate.

Soft Gelatin Capsules. A mixture of active ingredient is dissolved in a digestible oil such as soybean oil, cottonseed oil or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin. For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

The table below represents exemplary ranges for an oral suspension composition according to the present invention: The compound of the invention is accurately weighed and was transferred in 5-mL centrifuge tubes and the corresponding volume of each tested vehicle (shown in the table) was then added to reach 5 mg/mL as maximal solubility target concentration. Using a sonication bath the vials were firstly sonicated for 5-10 min and then transferred to a mixing and heating plate (Fisher Scientific Thermal Mixer—Isotemp) where the solutions were incubated at 25° C. for 2 hours and mixed at 500 rpm.

| Ingredients | Amount | |
|---|---|---|
| Compound 1 or 42 | 5-30 | mg/kg |
| Solutol HS15 | 0.2-0.4 | g |
| Vitamin E TPGS | 0.4 | g |
| Tween 80 | 0.1-0.4 | mL |
| Tetraglycol | 2-5 | mL |
| Methyl cellulose 400 cp | 0.1-0.5 | g |
| Water q.s | 15-30 | mL |

Gel Formulation.

The table below represents exemplary ranges for gel composition according to the present invention: Disperse the Carbomer 934 uniformly in about 40% of total amount of water. Add the ammonia solution gradually into the dispersion with agitation to form a clear gel. In a separate container dissolve the methyl paraben in prolylene glycol and then disperse 10.0 g of compound 15 in this solution to make a homogenous suspension. Gradually add the suspension into the gel with agitation, a uniform white opaque gel will be obtained.

| Ingredient | Amount | |
|---|---|---|
| Compound 11 | 10.0 | g |
| Carbomer 934 | 3.0 | g |
| Propylene Glycol | 40 | mL |
| Strong ammonia solution | 4.0 | mL |
| Methyl paraben | 3.0 | g |
| Purified water, USP q. s to | 1000 | g |

The table below represents exemplary ranges for cream or lotion composition according to the present invention: dissolve the methyl paraben in about 8000 of total amount of prolylene glycol. Add the poloxyl 2 cetyl ether into this solution with agitation. In a separate processing container mix the 20% of the propylene glycol, part of the purified water and 10.0 g of compound 15 to form a uniform suspension. Gradually add the suspension into the first processing container with moderate stirring until a homogenous, soft, white cream is obtained Pass the cream through a colloid mill and bring the mass of the batch to the targeted quantity.

| Ingredient | Amount | |
|---|---|---|
| Compound 11 | 10.0 | g |
| Poloxyl 2 cetyl ether | 50.0 | g |
| Propylene Glycol | 50 | mL |
| Methyl paraben | 3.0 | g |
| Purified water, USP q. s to | 1000 | g |

Non-Aerosol Spray Formulation:

The table below represents exemplary ranges for non-aerosol spray composition according to the present invention: 5.00 g of polyoxyl 10 oleyl ether is dissolved in 188.75 g of castor oil with gentle stirring. 25.0 g of compound 15 and 25.0 g of zinc oxide were suspended to the slurry with moderate stirring followed by 1.25 g of fumed silica gel. The slurry is mixed under high shear stress until uniform and smooth and packaged in a spray bottle.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

| Ingredient | Amount | |
|---|---|---|
| Compound 11 | 25.0 | g |
| Zinc oxide | 25.0 | g |
| Castor oil | 188.75 | g |
| Polyoxy 10 oleyl ether | 5 | g |

| | |
|---|---|
| Fumed silica | 1.25 |

In so far as the description above and the accompanying drawings disclose any additional subject matter, the inventions are not dedicated to the public and the right to file one or more applications to claim such additional inventions is reserved.

### REFERENCES BACKGROUND OF THE INVENTION

Lode H. Best use of antibiotics in nosocomial pneumonia improving efficacy and limiting resistance. Eur Respir Rev 2007; 16: 31-32.
American Thoracic Society, Infectious Diseases Society of America Guidelines for the management of adults with hospital acquired, ventilator-associated, and healthcare-associated pneumonia. Am J Respir Crit Care Med 2005; 171:388-416.
Rello J, Jubert P, Vall√©s J, Artigas A, Ru√© M, Niederman M S. Evaluation of outcome for intubated patients with pneumonia due to Pseudomonas aeruginosa. Clinical Infectious Diseases 1996; 23(5):973-978.
Prina E, Ranzani O T, Torres A. Community-acquired pneumonia. Lancet 2015; 386:1097-1108.
World Health Organization. The Top 10 Causes of Death. www.who.int/news-room/fact-sheets/detail/the-top-10-causes-of-death
Kollef M H, Shorr A, Tabak Y P, Gupta V, Liu L Z, Johannes R S. Epidemiology and outcomes of health-care-associated pneumonia: results from a large U S database of culture-positive pneumonia. Chest 2005; 128:3854-3862.
Restrepo M I, Anzueto A. The role of Gram-negative bacteria in healthcare-associated pneumonia. Semin Respir Crit Care Med 2009; 30:61-66.
Arancibia F, Bauer T T, Ewig S, Mensa J, Gonzalez J, Niederman M S, Torres A. Community-acquired pneumonia due to gram-negative bacteria and Pseudomonas aeruginosa: incidence, risk, and prognosis. Arch Intern Med 2002; 162:1849-1858.
Wunderink R G, Waterer G W. Community-acquired pneumonia. N Engl J Med 2014; 370:1863.
Lister P D, Wolter D J, Hanson N D. Antibacterial-resistant Pseudomonas aeruginosa: clinical impact and complex regulation of chromosomally encoded resistance mechanisms. Clin Microbiol Rev 2009; 22:582-610.
Jain S, Self W H, Wunderink R G, Fakhran S, Balk R, Bramley A M, Reed C, Grijalva C G, Anderson E J, Courtney D M, Chappell J D, Qi C, Hart E M, Carroll F, Trabue C, Donnelly H K, Williams D J, Zhu Y, Arnold S R, Ampofo K, Waterer G W, Levine M, Lindstrom S, Winchell J M, Katz J M, Erdman D, Schneider E, Hicks L A, McCullers J A, Pavia A T, Edwards K M, Finelli L; CDC EPIC Study Team. Community-acquired pneumonia requiring hospitalization among U.S. adults. N Engl J Med 2015; 373:415-427.
Govindaraj Vaithinathan A, Vanitha A. WHO global priority pathogens list on antibiotic resistance: an urgent need for action to integrate One Health data. Perspectives in Public Health 2018; 138(2):87-88.
Tamma P D, Cosgrove S E, Maragakis L L. Combination Therapy for Treatment of Infections with Gram-Negative Bacteria. Clin Microbiol Rev. 2012; 25(3):450-470.
Hancock R E W, Nijnik A, Philpott D J Modulating immunity as a therapy for bacterial infections. Nature Reviews Microbiology 2012; 10(4):243-254.
Faure K, Sawa T, Ajayi T, Fujimoto J, Moriyama K, Shime N, Wiener-Kronish J P. TLR4 signaling is essential for survival in acute lung injury induced by virulent Pseudomonas aeruginosa secreting type III secretory toxins. Respiratory Research 2004; 5(1):1-1.

### REFERENCES

Leon, Carlos G.; Tory, Rita; Jia, Jessica; Sivak, Olena; Wasan, Kishor M.
Pharmaceutical Research (2008), 25(8), 1751-1761.
Savva Athina; Roger Thierry From Frontiers in immunology (2013), 4387, Language: English, Database: MEDLINE
Cho, Y.; Wang, J. J.; Chew, E. Y.; Ferris, F. L.; Mitchell, P.; Chan, C.-C.; Tuo, J., Toll-like Receptor Polymorphisms and Age-Related Macular Degeneration: Replication in Three Case-Control Samples. Investigative Ophthalmology & Visual Science 2009, 50, (12), 5614-5618.
Higgins, G. T.; Wang, J. H.; Dockery, P.; Cleary, P. E.; Redmond, H. P., Induction of angiogenic cytokine expression in cultured RPE by ingestion of oxidized photoreceptor outer segments. Invest Ophthalmol Vis Sci. 2003, 44(4):1775-82
Kaarniranta, K.; Salminen, A., Age-related macular degeneration: activation of innate immunity system via pattern recognition receptors. J Mol Med (Berl). 2009, 87(2):117-23.
Elner, S. G.; Petty, H. R.; Elner, V. M.; Yoshida, A.; Bian, Z.-M.; Yang, D.; Kindezelskii, A. L., TLR4 mediates human retinal pigment epithelial endotoxin binding and cytokine expression. Transactions of the American Ophthalmological Society 2005, 103, 126-137.
Yi, H.; Patel, A. K.; Sodhi, C. P.; Hackam, D. J.; Hackam, A. S., Novel Role for the Innate Immune Receptor Toll-Like Receptor 4 (TLR4) in the Regulation of the Wnt Signaling Pathway and Photoreceptor Apoptosis. PLoS One 2012, 7, (5), e36560.
He, C.; Sun, Y.; Ren, X.; Lin, Q.; Hu, X.; Huang, X.; Su, S.-B.; Liu, Y.; Liu, X., Angiogenesis mediated by toll-like receptor 4 in ischemic neural tissue. Arterioscler Thromb Vasc Biol. 2013, 33(2):330-8.
Huang, J.-D.; Amaral, J.; Lee, J. W.; Rodriguez, I. R., 7-Ketocholesterol-Induced Inflammation Signals Mostly through the TLR4 Receptor Both In Vitro and In Vivo. PLoS One 2014, 9, (7), e100985.
CN 102475714.
CN 101732338.
US 20020022601.
Hadwiger, Lee A.; Klosterman, S.; Chang, M.-M.; Friel, P.; Hosick, H. L., From Advances in Chitin Science (1997), 2, 102-109.
EP 183556.
Xu, Ke; Chen, Jun-quan Zhongguo Laonianxue Zazhi (2012), 32(24), 5478-5480.
Wang, Jianyun; Chen, Yuanwei; Ding, Yulong; Shi, Guoqi; Wan, Changxiu, Applied Surface Science (2008), 255(2), 260-262.
Xiong, Chuannan; Wu, Haige; Wei, Peng; Pan, Ma; Tuo, Yaqin; Kusakabe, Isao; Du, Yuguang, Carbohydrate Research (2009), 344(15), 1975-1983.
CN102085212.
Opal, S. M.; Laterre, P.; Francois, B.; et al., Effect of eritoran, an antagonist of md2-tlr4, on mortality in patients with severe sepsis: The access randomized trial. JAMA 2013, 309, 1154-1162.
Wong, W. L.; Su, X.; Li, X.; Cheung, C. M. G.; Klein, R.; Cheng, C.-Y.; Wong, T. Y., Global prevalence of age-related macular degeneration and disease burden projection for 2020 and 2040: a systematic review and meta-analysis. The Lancet Global Health 2014, 2, e106-e116.
Harrell, S. N.; Brandon, D. H., Retinopathy of Prematurity: The Disease Process, Classifications, Screening, Treatment, and Outcomes. Neonatal Network 2007, 26, 371-378.
Cho, Y.; Wang, J. J.; Chew, E. Y.; Ferris, F. L.; Mitchell, P.; Chan, C.-C.; Tuo, J., Toll-like Receptor Polymorphisms and Age-Related Macular Degeneration: Replication in Three Case-Control Samples. Investigative Ophthalmology & Visual Science 2009, 50, 5614-5618.
He, C.; Sun Y Fau-Ren, X.; Ren X Fau-Lin, Q.; Lin Q Fau-Hu, X.; Hu X Fau-Huang, X.; Huang X Fau-Su, S.-B.; Su Sb Fau-Liu, Y.; Liu Y Fau-Liu, X.; Liu, X., Angiogenesis mediated by toll-like receptor 4 in ischemic neural tissue.
Xia, W.; Liu, P.; Zhang, J.; Chen, J., Biological activities of chitosan and chitooligosaccharides. Food Hydrocolloids 2011, 25, 170-179.
Mosser, D. M., The many faces of macrophage activation. Journal of Leukocyte Biology 2003, 73, 209-212.
Panda, S. K.; Kumar, S.; Tupperwar, N. C.; Vaidya, T.; George, A.; Rath, S.; Bal, V.; Ravindran, B., Chitohexaose Activates Macrophages by Alternate Pathway through TLR4 and Blocks Endotoxemia. PLoS Pathog 2012, 8, e1002717.
Roger, T.; Froidevaux C Fau-Le Roy, D.; Le Roy D Fau-Reymond, M. K.; Reymond Mk Fau-Chanson, A.-L.; Chanson Al Fau-Mauri, D.; Mauri D Fau-Burns, K.; Burns K Fau-Riederer, B. M.; Riederer Bm Fau-Akira, S.; Akira S Fau-Calandra, T.; Calandra, T., Protection from lethal gram-negative bacterial sepsis by targeting Toll-like receptor 4.
Ceri, H.; Olson, M. E.; Stremick, C.; Read, R. R.; Morck, D.; Buret, A., The Calgary Biofilm Device: New Technology for Rapid Determination of Antibiotic Susceptibilities of Bacterial Biofilms. Journal of Clinical Microbiology 1999, 37, 1771-1776.
Li, L.; Cheung, A.; Bayer, A. S.; Chen, L.; Abdelhady, W.; Kreiswirth, B. N.; Yeaman, M. R.; Xiong, Y. Q., The Global Regulon sarA Regulates beta-lactam Antibiotic Resistance In Methicillin-resistant Staphylococcus aureus (MRSA) In Vitro and In Endovascular Infections. The Journal of infectious diseases 2016.
Archer, N. K.; Mazaitis, M. J.; Costerton, J. W.; Leid, J. G.; Powers, M. E.; Shirtliff, M. E., Staphylococcus aureus biofilms: Properties, regulation and roles in human disease. Virulence 2011, 2, 445-459.
Hurdle, J. G.; Yendapally, R.; Sun, D.; Lee, R. E., Evaluation of Analogs of Reutericyclin as Prospective Candidates for Treatment of Staphylococcal Skin Infections. Antimicrobial Agents and Chemotherapy 2009, 53, 4028-4031.
Hurdle, J. G.; Lee, R. B.; Budha, N. R.; Carson, E. I.; Qi, J.; Scherman, M. S.; Cho, S. H.; McNeil, M. R.; Lenaerts, A. J.; Franzblau, S. G.; Meibohm, B.; Lee, R. E., A microbiological assessment of novel nitrofuranylamides as anti-tuberculosis agents. Journal of Antimicrobial Chemotherapy 2008, 62, 1037-1045.
Spilsbury, K.; Garrett, K. L.; Shen, W.-Y.; Constable, I. J.; Rakoczy, P. E., Overexpression of Vascular Endothelial Growth Factor (VEGF) in the Retinal Pigment Epithelium Leads to the Development of Choroidal Neovascularization. The American Journal of Pathology 2000, 157, 135-144.
Betts, B. S.; Parvathaneni, K.; Yendluri, B. B.; Grigsby, J.; Tsin, A. T. C., Ginsenoside-Rb1 Induces ARPE-19 Proliferation and Reduces VEGF Release. ISRN Ophthalmology 2011, 2011, 184295.
Balany, J. and V. Bhandari (2015). “Understanding the Impact of Infection, Inflammation, and Their Persistence in the Pathogenesis of Bronchopulmonary Dysplasia.” Front Med (Lausanne) 2: 90.
Bancalari, E. and N. Claure (2016). Bronchopulmonary dysplasia: definitions and epidemiology. Bronchopulmonary Dysplasia. V. Bhandari. Switzerland, Springer International Publishing: 167-182.
Bhandari, A. and V. Bhandari (2009). “Pitfalls, problems, and progress in bronchopulmonary dysplasia.” Pediatrics 123(6): 1562-1573.
Bhandari, A. and V. Bhandari (2011). “New” Bronchopulmonary Dysplasia: A clinical review.” Clin Pulm Med 18(3): 137-143.
Bhandari, A. and S. McGrath-Morrow (2013). “Long-term pulmonary outcomes of patients with bronchopulmonary dysplasia.” Semin Perinatol 37(2): 132-137.
Bhandari, V. (2008). “Molecular mechanisms of hyperoxia-induced acute lung injury.” Front Biosci 13: 6653-6661.
Bhandari, V. (2014). “Drug therapy trials for the prevention of bronchopulmonary dysplasia: current and future targets.” Front Pediatr 2: 76.
Bhandari, V. (2014). “Postnatal inflammation in the pathogenesis of bronchopulmonary dysplasia.” Birth Defects Res A Clin Mol Teratol 100(3): 189-201.
Bhandari, V., R. Choo-Wing, C. G. Lee, K. Yusuf, J. H. Nedrelow, N. Ambalavanan, H. Malkus, R. J. Homer and J. A. Elias (2008). “Developmental regulation of NO-mediated VEGF-induced effects in the lung.” Am J Respir Cell Mol Biol 39(4): 420-430.
Bi, M. H., B. E. Wang, X. X. Zheng, M. Li, K. Mayer and S. W. Zhang (2008). “[The effect of recombinant interleukin-10/Fc fusion protein on lipopolysaccharide-induced acute lung injury in mice].” Zhongguo Wei Zhong Bing Ji Jiu Yi Xue 20(8): 461-464.
Choo-Wing R, S. M., Harijith A, Bowen B, Pryhuber G, Janer C, Andersson S, Homer R J, Bhandari V (2013). “Hyperoxia and interferon-7-induced injury in developing lungs occur via cyclooxygenase-2 and the endoplasmic reticulum stress-dependent pathway” Am J Respir Cell Mol Biol 48(6): 749-757.
Dai, C., X. Yao, E. M. Gordon, A. Barochia, R. A. Cuento, M. Kaler, K. S. Meyer, K. J. Keeran, G. Z. Nugent, K. R. Jeffries, X. Qu, Z. X. Yu, A. Aponte, M. Gucek, P. K. Dagur, J. P. McCoy and S. J. Levine (2016). “A CCL24-dependent pathway augments eosinophilic airway inflammation in house dust mite-challenged Cd163−/− mice.” Mucosal Immunology 9(3): 702-717.
Densmore, J. C., P. R. Signorino, J. Ou, O. A. Hatoum, J. J. Rowe, Y. Shi, S. Kaul, D. W. Jones, R. E. Sabina, K. A. Pritchard, Jr., K. S. Guice and K. T. Oldham (2006). “Endothelium-derived microparticles induce endothelial dysfunction and acute lung injury.” Shock 26(5): 464-471.
Harijith, A. and V. Bhandari (2016). Hyperoxia in the pathogenesis of bronchopulmonary dysplasia. Bronchopulmonary Dysplasia. V. Bhandari. Switzerland, Springer International Publishing: 3-26.
Jensen, E. A. and B. Schmidt (2014). “Epidemiology of bronchopulmonary dysplasia.” Birth Defects Res A Clin Mol Teratol 100(3): 145-157.
Kapur, R., M. Kim, J. Rebetz, M. T. Rondina, L. Porcelijn and J. W. Semple (2017). “Low levels of interleukin-10 in patients with transfusion-related acute lung injury.” Annals of Translational Medicine 5(16): 339.
Kneidl, J., B. Loffler, M. C. Erat, J. Kalinka, G. Peters, J. Roth and K. Barczyk (2012). “Soluble CD163 promotes recognition, phagocytosis and killing of Staphylococcus aureus via binding of specific fibronectin peptides.” Cell Microbiol 14(6): 914-936.
Kwon, J. H., M. Kim, Y. K. Bae, G.-H. Kim, S. J. Choi, W. Oh, S. Um and H. J. Jin (2019). “Decorin Secreted by Human Umbilical Cord Blood-Derived Mesenchymal Stem Cells Induces Macrophage Polarization via CD44 to Repair Hyperoxic Lung Injury.” International journal of molecular sciences 20(19): 4815.
Lee, H.-S. and C.-K. Kim (2011). “Effect of recombinant IL-10 on cultured fetal rat alveolar type II cells exposed to 65%-hyperoxia.” Respiratory Research 12(1): 68-68.
Li, H. D., Q. X. Zhang, Z. Mao, X. J. Xu, N. Y. Li and H. Zhang (2015). “Exogenous interleukin-10 attenuates hyperoxia-induced acute lung injury in mice.” Exp Physiol 100(3): 331-340.
Li, Z., R. Choo-Wing, H. Sun, A. Sureshbabu, R. Sakurai, V. K. Rehan and V. Bhandari (2011). “A potential role of the JNK pathway in hyperoxia-induced cell death, myofibroblast transdifferentiation and TGF-beta1-mediated injury in the developing murine lung.” BMC Cell Biol 12: 54.
McEvoy, C. T., L. Jain, B. Schmidt, S. Abman, E. Bancalari and J. L. Aschner (2014). “Bronchopulmonary dysplasia: NHLBI Workshop on the Primary Prevention of Chronic Lung Diseases.” Ann Am Thorac Soc 11 Suppl 3: S146-153.
Moore, K. W., F. Rousset and J. Banchereau (1991). “Evolving principles in immunopathology: interleukin 10 and its relationship to Epstein-Barr virus protein BCRF1.” Springer Semin Immunopathol 13(2): 157-166.
Natarajan, G., A. Pappas, S. Shankaran, D. E. Kendrick, A. Das, R. D. Higgins, A. R. Laptook, E. F. Bell, B. J. Stoll, N. Newman, E. C. Hale, R. Bara and M. C. Walsh (2012). “Outcomes of extremely low birth weight infants with bronchopulmonary dysplasia: impact of the physiologic definition.” Early Hum Dev 88(7): 509-515.
Raju, T. N. K., A. S. Buist, C. J. Blaisdell, M. Moxey-Mims and S. Saigal (2017). “Adults born preterm: a review of general health and system-specific outcomes.” Acta Paediatr 106(9): 1409-1437.
Rubenfeld, G. D., E. Caldwell, E. Peabody, J. Weaver, D. P. Martin, M. Neff, E. J. Stern and L. D. Hudson (2005). “Incidence and outcomes of acute lung injury.” N Engl J Med 353(16): 1685-1693.
Russell, R. B., N. S. Green, C. A. Steiner, S. Meikle, J. L. Howse, K. Poschman, T. Dias, L. Potetz, M. J. Davidoff, K. Damus and J. R. Petrini (2007). “Cost of hospitalization for preterm and low birth weight infants in the United States.” Pediatrics 120(1): el-9.
Salim, A., M. Martin, C. Constantinou, B. Sangthong, C. Brown, G. Kasotakis, D. Demetriades and H. Belzberg (2006). “Acute respiratory distress syndrome in the trauma intensive care unit: Morbid but not mortal.” Arch Surg 141(7): 655-658.
Smith, V. C., J. A. Zupancic, M. C. McCormick, L. A. Croen, J. Greene, G. J. Escobar and D. K. Richardson (2005). “Trends in severe bronchopulmonary dysplasia rates between 1994 and 2002.” J Pediatr 146(4): 469-473.
Sun H, C.-W. R., Fan J, Leng L, Syed M A, Hare A A, Jorgensen W L, Bucala R, Bhandari V (2013). “Small molecular modulation of macrophage migration inhibitory factor in the hyperoxia-induced mouse model of bronchopulmonary dysplasia.” Respir Res 14(1).
Sun H, C.-W. R., Sureshbabu A, Fan J, Leng L, Yu S, Jiang D, Noble P, Homer R J, Bucala R, Bhandari V (2013). “A critical regulatory role for macrophage migration inhibitory factor in hyperoxia-induced injury in the developing murine lung.” PLoS ONE 8(4).
Sureshbabu, A., M. Syed, P. Das, C. Janer, G. Pryhuber, A. Rahman, S. Andersson, R. J. Homer and V. Bhandari (2016). “Inhibition of RPTOR Prevents Hyperoxia-induced Lung Injury by Enhancing Autophagy and Reducing Apoptosis in Neonatal Mice.” Am J Respir Cell Mol Biol.
Sureshbabu, A., M. A. Syed, C. S. Boddupalli, M. V. Dhodapkar, R. J. Homer, P. Minoo and V. Bhandari (2015). “Conditional overexpression of TGFbeta1 promotes pulmonary inflammation, apoptosis and mortality via TGFbetaR2 in the developing mouse lung.” Respir Res 16(1): 4.
Syed, M. A., R. Choo-Wing, R. J. Homer and V. Bhandari (2016). “Role of Nitric Oxide Isoforms in Vascular and Alveolar Development and Lung Injury in Vascular Endothelial Growth Factor Overexpressing Neonatal Mice Lungs.” PLoS One 11(1): e0147588.
Timmermann, M. and P. Hogger (2005). “Oxidative stress and 8-iso-prostaglandin F(2alpha) induce ectodomain shedding of CD163 and release of tumor necrosis factor-alpha from human monocytes.” Free Radic Biol Med 39(1): 98-107.
Trembath, A. and M. M. Laughon (2012). “Predictors of bronchopulmonary dysplasia.” Clin Perinatol 39(3): 585-601.
Villar, J., J. Blanco and R. M. Kacmarek (2016). “Current incidence and outcome of the acute respiratory distress syndrome.” Curr Opin Crit Care 22(1): 1-6.
Weaver, L. K., P. A. Pioli, K. Wardwell, S. N. Vogel and P. M. Guyre (2007). “Up-regulation of human monocyte CD163 upon activation of cell-surface Toll-like receptors.” J Leukoc Biol 81(3): 663-671.
Zhi, Y., P. Gao, X. Xin, W. Li, L. Ji, L. Zhang, X. Zhang and J. Zhang (2017). “Clinical significance of sCD163 and its possible role in asthma (Review).” Mol Med Rep 15(5): 2931-2939.

What is claimed is:

1. A pharmaceutical composition comprising a compound according to Formula (I), or a pharmaceutically acceptable salt thereof: 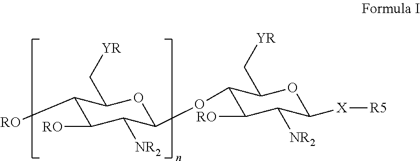
R=H, C(O)R₁, alkyl, benzyl, substituted benzyl;
R₁=CH₃, alkyl, piperidine nitroxyl, or biotin;
R₂=H, C(O)R¹, C(S)NR¹ or aceloxy alkyl carbamate of the following formula:
R₂=C(O)OCHR₃OC(O)OR₄, piperidine nitroxyl, or fluorescein isothiocyanate (FITC);
R₃=H, CH₃, C₂H₅, isopropyl;
R₄=substituted alkyl group;
X=H, O, NH, or S and is linked to the anomeric carbon via R stereochemistry (beta anomer) at a pH of 6.5-7.4;
Y=O, NH or S;
R₅=aryl, substituted aryl, heteroaryl, substituted heteroaryl, cycloalkyls and substituted cycloalkyl, piperidine nitroxyl, piperidine N-hydroxylamine, 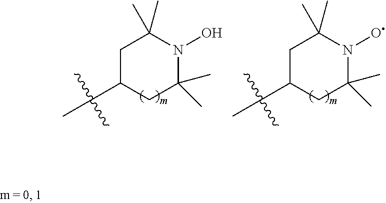
wherein the substituted aryl groups are defined as below 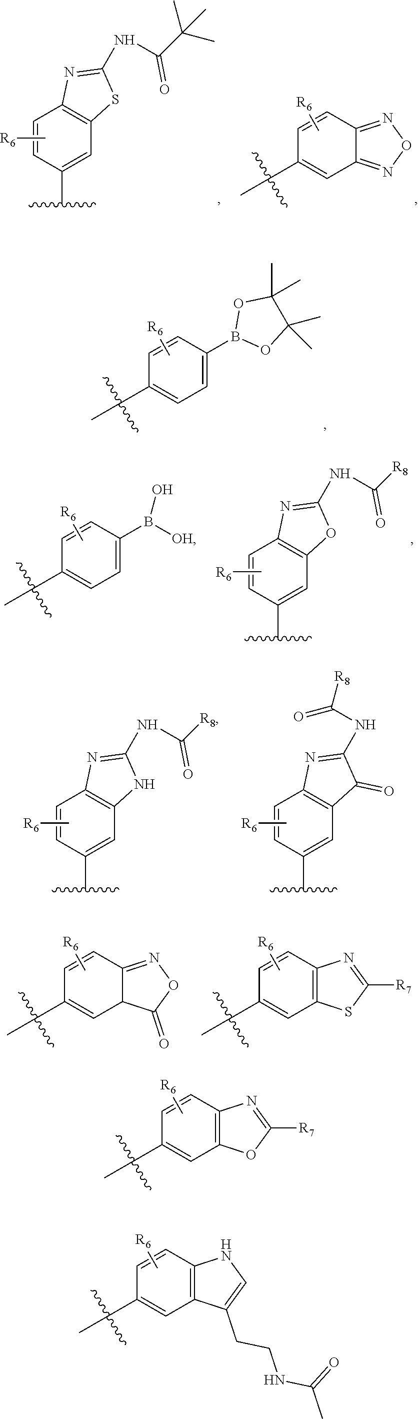 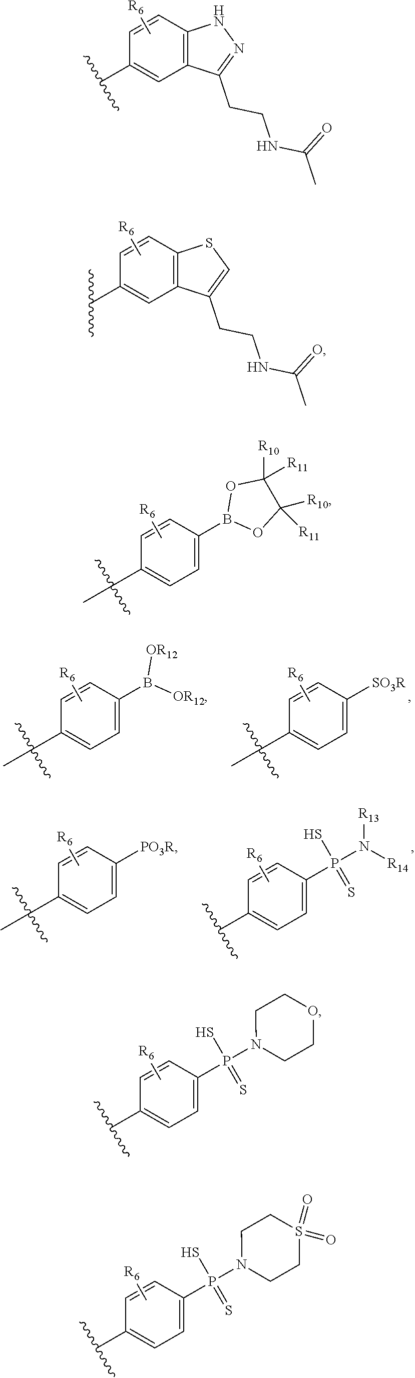 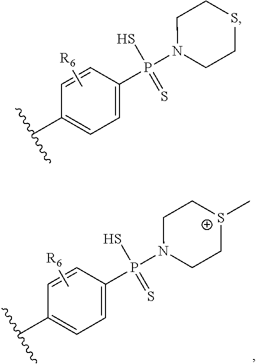
R₆=H, NR, OR₃, SR₃, Cl, Br, F, I, NO₂, CO₂H, CO₂R₃
R₇ to R₁₁ are selected from: H, acyl, alkyl, substituted alkyl, alkenyl, alkynyl, alkoxy, aryloxy, alkoxycarbonyl, amido, amino, carbonate, carbamate, carbonyl, ester, halo, hydroxy, phosphate, phosphonate, phosphinate, phosphine oxide, urea, cycloalkyl, heterocycloalkyl, aryl, and heteroaryl, C(O)R¹; and
n=0-7.

2. The composition of claim 1, wherein the composition is formulated a sterile, injectable aqueous or oleaginous suspension.

3. The composition of claim 1, wherein the composition is formulated as a sterile topical gel, ointment or aqueous spray.

4. The composition of claim 1, further comprising an anti-inflammatory agent, an antimicrobial agent, or both.

5. The composition of claim 1, wherein the compound of Formula (I) is further defined as:

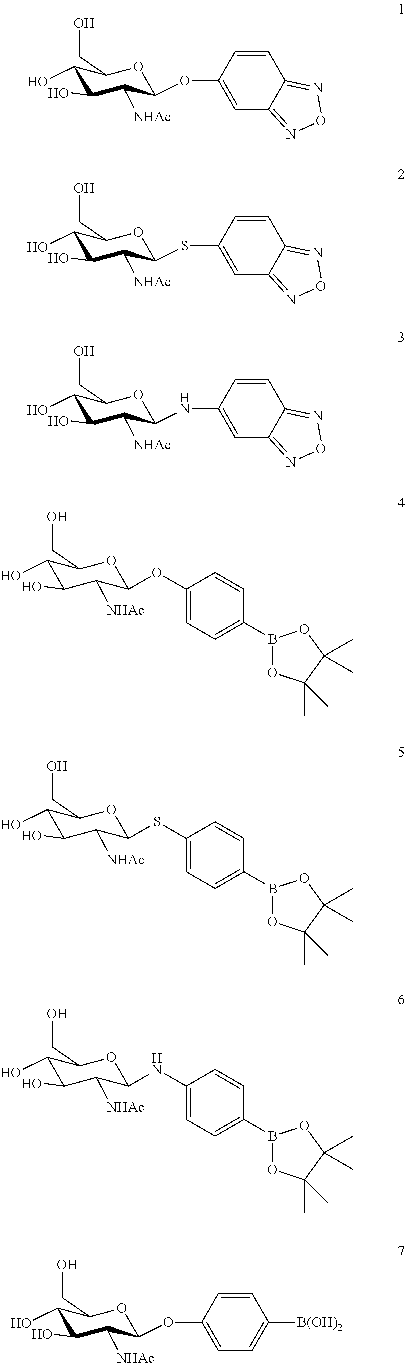

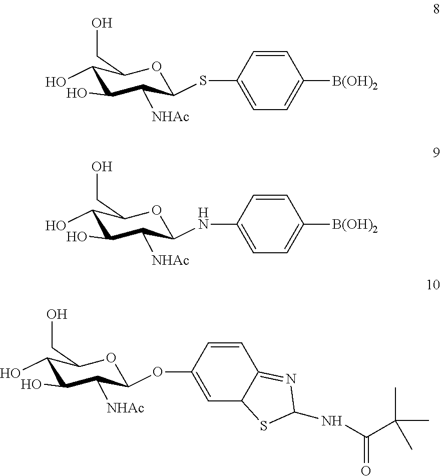

6. The composition of claim 1, wherein the compound of Formula (I) is further defined as:
n=0, X=NH, Y=N, R=Biotin, R₂=C(O)CH₃, R₅=aryl, substituted aryl or substituted heteroaryl.

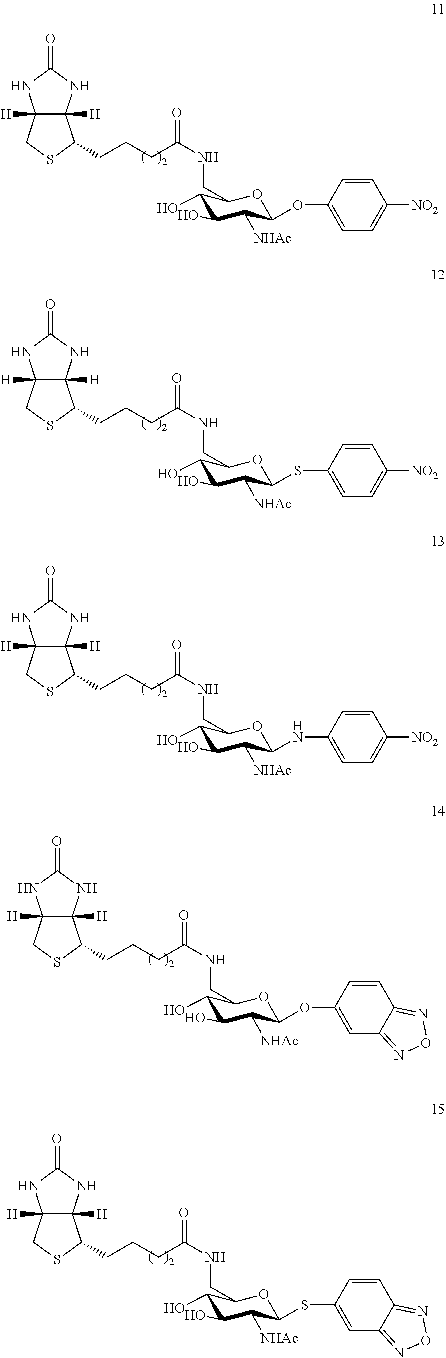

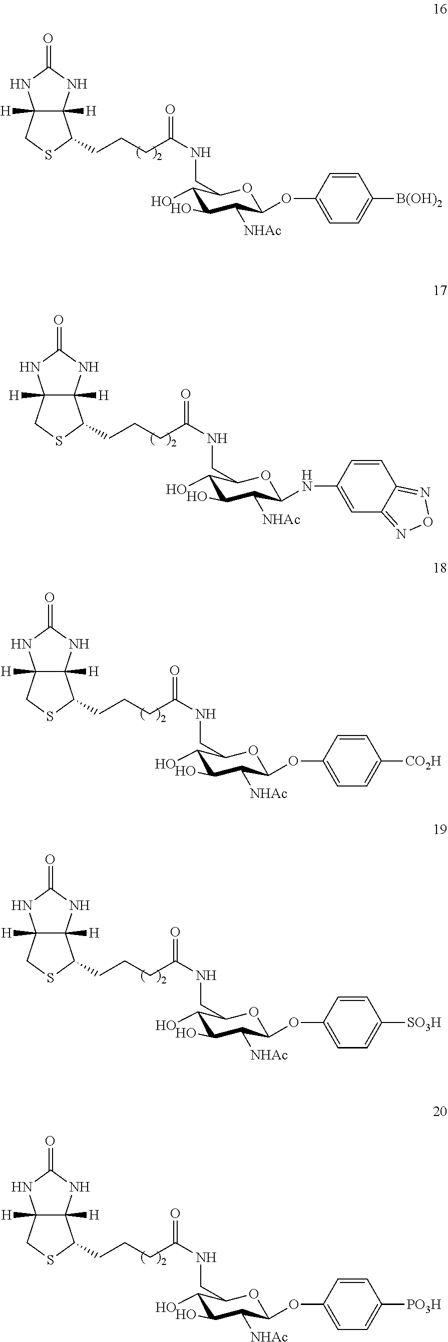

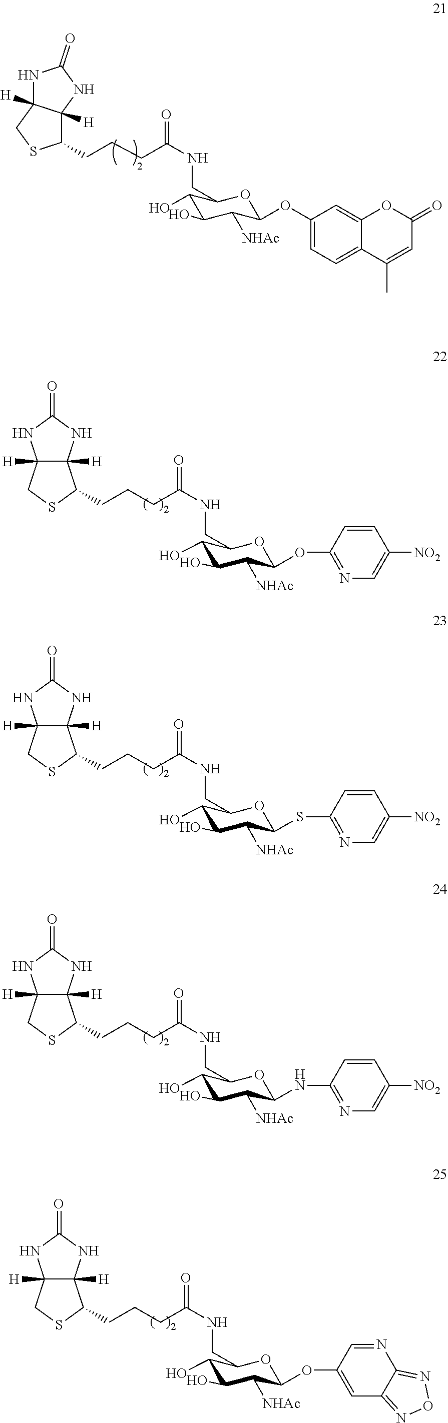

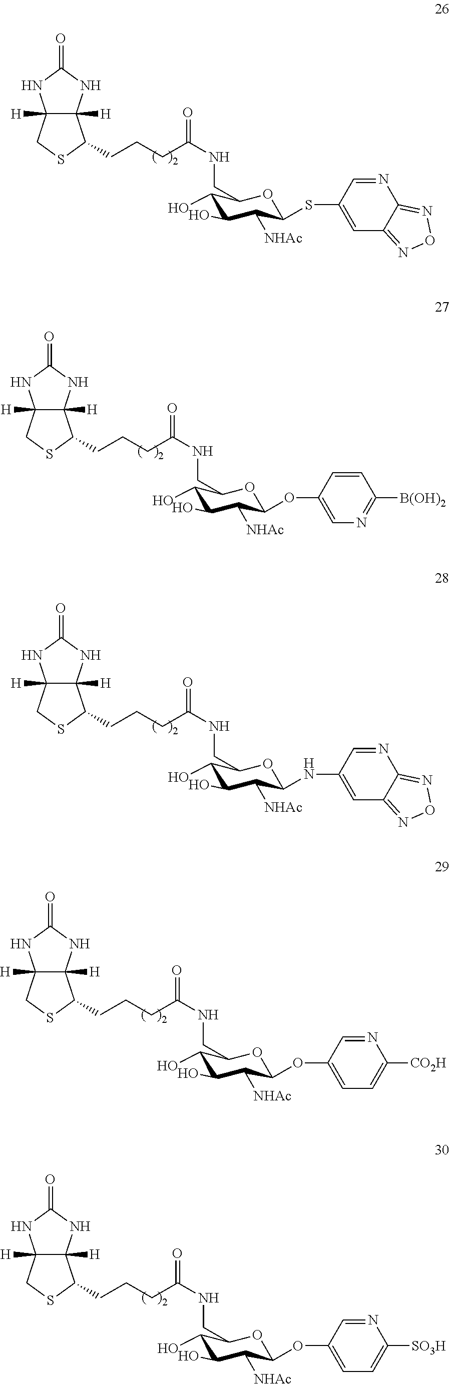

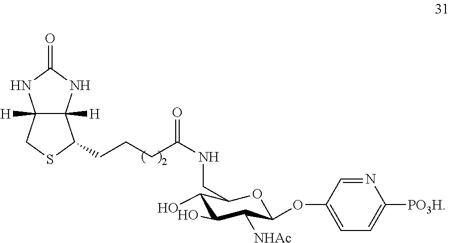

7. The composition of claim 1, wherein the compound of Formula (I) is further defined as:
n=0, X=NH, Y=N, R=Biotin, R₂=C(O)CH₃, R₅=cycloalkyl or heterocycloalkyl

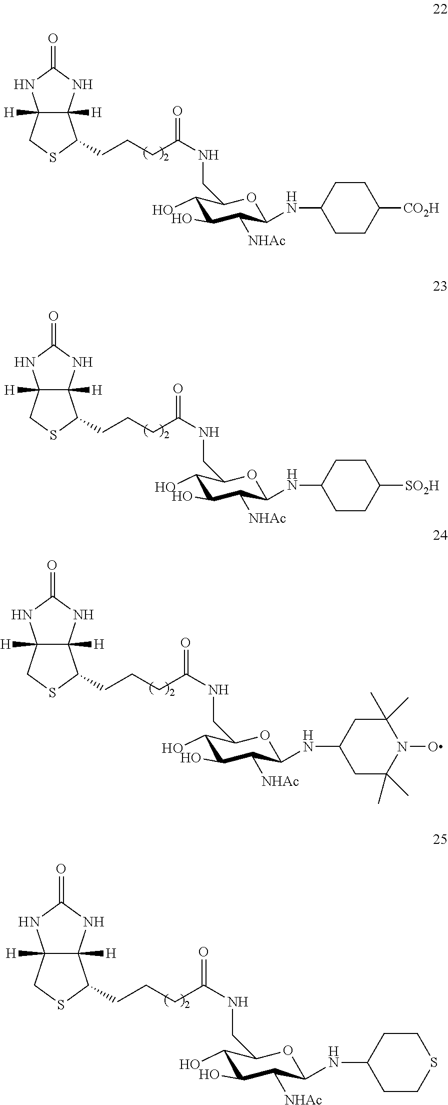

8. The composition of claim 1, wherein the compound of Formula (I) is further defined as:
n=0, X=O, Y=O, R=H, R₂=FITC, R₅=aryl or substituted aryl

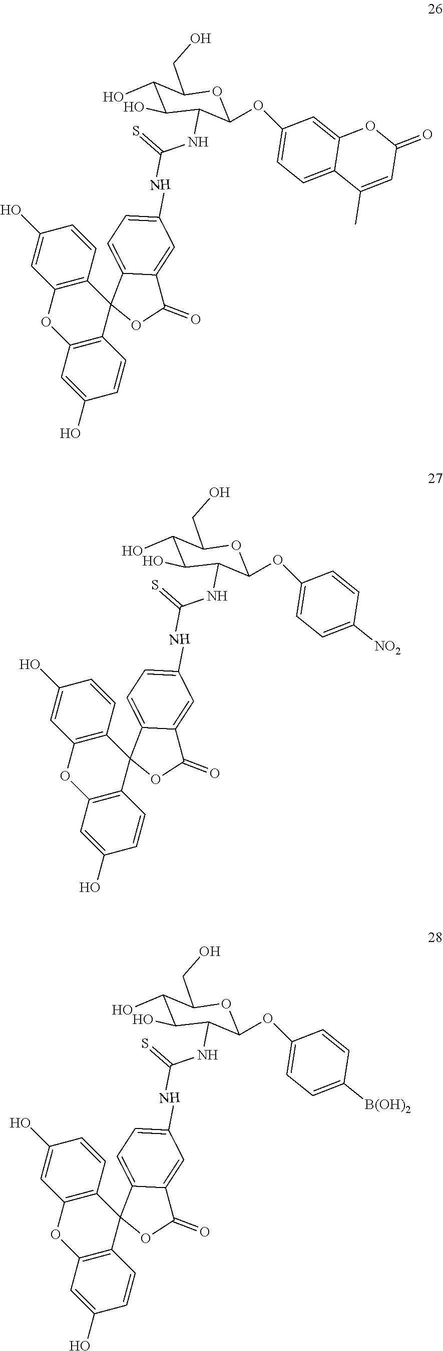 [SMILES: Cc1cc(=O)oc2cc(OC3OC(CO)C(O)C(O)C3NC(=S)Nc3ccc4c(c3)C(=O)OC43c4ccc(O)cc4Oc4cc(O)ccc43)ccc12 ; O=C1OC2(c3ccc(O)cc3Oc3cc(O)ccc32)c2ccc(NC(=S)NC3C(Oc4ccc([N+](=O)[O-])cc4)OC(CO)C(O)C3O)cc21 ; O=C1OC2(c3ccc(O)cc3Oc3cc(O)ccc32)c2ccc(NC(=S)NC3C(Oc4ccc(B(O)O)cc4)OC(CO)C(O)C3O)cc21]

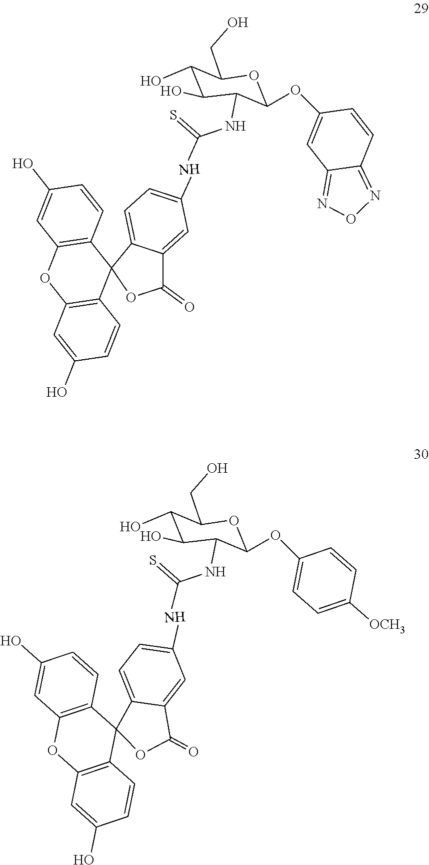 [SMILES: O=C1OC2(c3ccc(O)cc3Oc3cc(O)ccc32)c2ccc(NC(=S)NC3C(Oc4ccc5nonc5c4)OC(CO)C(O)C3O)cc21 ; COc1ccc(OC2OC(CO)C(O)C(O)C2NC(=S)Nc2ccc3c(c2)C(=O)OC32c3ccc(O)cc3Oc3cc(O)ccc32)cc1]

9. The composition of claim 1, wherein the compound of Formula (I) is further defined as:
n=2-7, X=OH, Y=O, R=H, R₂=H, C(O)CH₃, FITC, or piperidine nitroxyl, R₅=H, cyclohexyl, heterocycloakyl

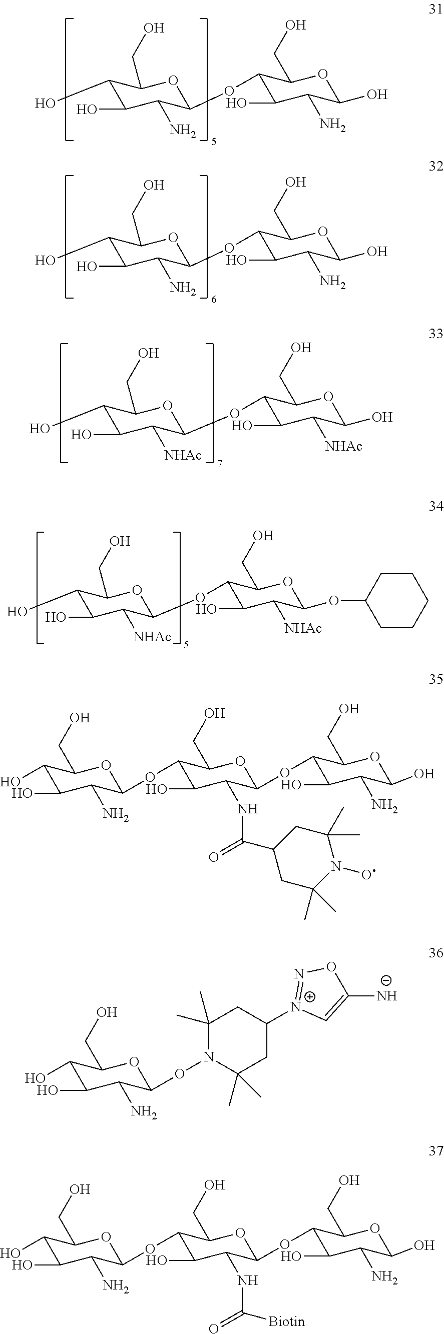

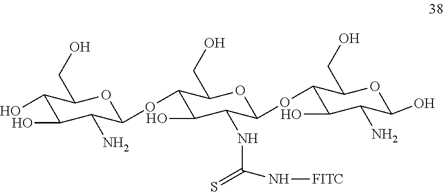

10. A method of treating at least one of sepsis, septicemia, septic shock, peritonitis, skin and soft tissue infections, ocular infection, ocular inflammation, ocular angiogenesis, rheumatoid arthritis (RA), atherosclerosis, inflammatory bowel diseases (IBD), necrotizing enterocolitis, asthma, chronic obstructive pulmonary disease, acute respiratory distress syndrome, acute lung injury, bacterial and viral pneumonia, chronic lung injury, acute kidney injury, chronic kidney injury, fibrosis, fever syndromes, cachexia, psoriasis, autoimmune diseases, cardiac diseases, retinoblastoma, cancer, disorder associated with inflammation, immunomodulation or microbial infections that comprises administering to a subject in need thereof a therapeutically effective amount of a pharmaceutical composition according to claim 1, or a pharmaceutically acceptable salt thereof, whereby the subject is treated.

11. The method of claim 10, wherein the step of administering comprises administering the pharmaceutical composition comprising about 5.0 mg to about 100 mg of a compound according to Formula (I), or a pharmaceutically acceptable salt thereof to a patient in need thereof, whereby the patient is treated.

12. The method of claim 10, wherein the step of administering comprises providing the pharmaceutical composition comprising about 10.0 mg to about 1000 mg of a compound according to Formula (I), or a pharmaceutically acceptable salt thereof to a patient in need thereof, whereby said patient is treated.

13. A composition comprising an effective amount of a compound according to Formula (I), or a pharmaceutically acceptable salt thereof: 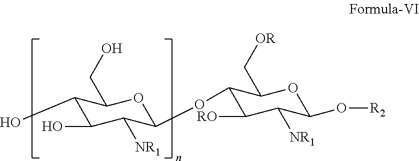
where:
n=0-1
R=benzyl, substituted benzyl
R₁=COCH₃, N-dimethylmaleimide
R₂=cyclohexyl, p-nitro phenyl, piperidine nitroxy, piperidine-N-hydroxyl, p-methoxy phenyl, and a pharmaceutically acceptable excipient.

14. The composition of claim 13, wherein the composition is formulated a sterile, injectable aqueous or oleaginous suspension.

15. The composition of claim 13, wherein the composition is formulated as a sterile topical ocular solution.

16. The composition of claim 13, wherein the compound is selected from:

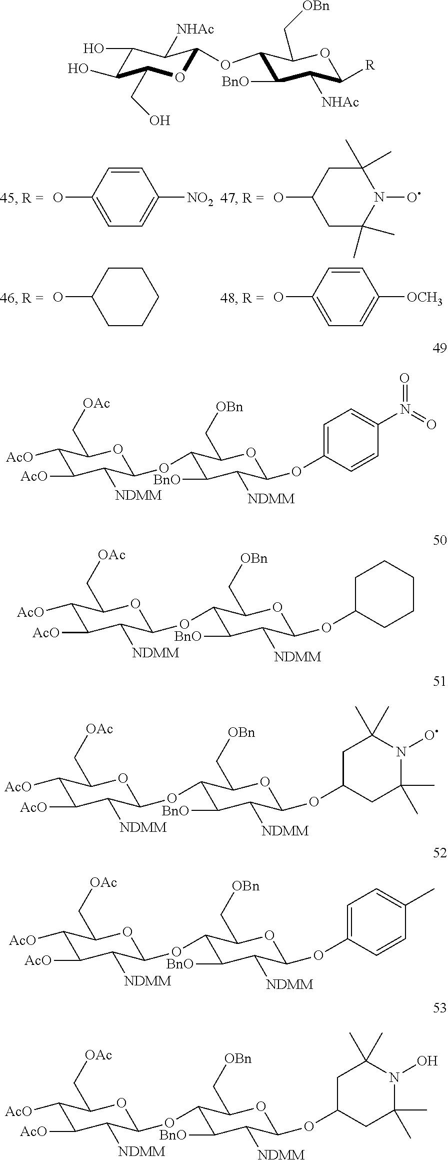

17. A method of treating ocular angiogenesis, ocular inflammation which comprises administering to a subject in need thereof a therapeutically effective amount of a pharmaceutical composition according to claim 13, or a pharmaceutically acceptable salt thereof, whereby said subject is treated.

18. The method of claim 17, wherein the compounds are selected from at least one of compounds 38 to 44: [chemical structure]
